(19) United States
(12) Patent Application Publication
(10) Pub. No.: US 2026/0207731 A1
(43) Pub. Date: Jul. 23, 2026

(54) LIPID NANOPARTICLE COMPOSITION

(71) Applicant: SEQIRUS INC., Holly Springs, NC (US)

(72) Inventors: Jonathan NOLASCO, Holly Springs, NC (US); Ghazal HARRIRI, Holly Springs, NC (US)

(21) Appl. No.: 19/140,123

(22) PCT Filed: Dec. 20, 2023
(86) PCT No.: PCT/IB2023/062968

§ 371 Date: Jun. 17, 2025

Related U.S. Application Data
(60) Provisional application No. 63/476,217, filed on Dec. 20, 2022.

(51) Int. Cl. A61K 39/145; A61K 9/1272; A61K 9/51; A61K 39/00; A61K 47/69; A61P 31/16; B82Y 5/00; C12N 7/00; C12N 15/88

(52) U.S. Cl. CPC A61K 39/145; A61K 9/1272; A61K 9/5123; A61K 9/5192; A61K 47/6929; A61P 31/16; C12N 7/00; C12N 15/88; A61K 2039/53; A61K 2039/55555; A61K 2039/572; A61K 2039/575; B82Y 5/00; C12N 2760/16134

(57) ABSTRACT

The present disclosure relates to compositions of RNA encapsulated in lipid nanoparticles and methods of producing the same. The compositions can be used for delivery of mRNA to a subject. The lipid nanoparticles have improved properties for delivery of biologically active agents, such as RNA.

### RELATED APPLICATION DATA

The present application claims priority from U.S. Provisional Patent Application No. 63/476,217 filed on 20 Dec. 2022 entitled “Lipid nanoparticle composition”, the entire contents of which is incorporated herein by reference in its entirety.

### SEQUENCE LISTING

The present application is filed together with a Sequence Listing in electronic format. The entire contents of the Sequence Listing are hereby incorporated by reference.

### FIELD

The present disclosure relates compositions of mRNA encapsulated in lipid nanoparticles and methods of producing the same. The compositions can be used for delivery of mRNA to a subject.

### BACKGROUND

Nucleic acid-based therapies have shown substantial promise in a range of therapeutic applications. The delivery of polynucleotides such as messenger RNA (mRNA), small interfering RNA (siRNA), antisense oligonucleotides, plasmids, DNA and the like does, however, present a number of challenges. Free nucleic acids, such as RNAs, are subject to rapid enzymatic degradation and so generally do not persist systemically. Additionally, due to their negative charge the nucleic acids may not be able to effectively cross the cellular barriers to enter the necessary intracellular compartment, for example, for translation or to otherwise achieve their effect. This is particularly the case for mRNA, which can be a very large molecule with a high negative charge density. mRNA is also highly prone to degradation by 5′ exonucleases, 3′ exonucleases, and endonucleases and is an inherently unstable molecule.

Lipid nanoparticles (LNPs) have therefore been used to formulate nucleic acids so as to protect them from degradation and improve cellular uptake and intracellular delivery. LNPs are commonly formed from ionizable cationic lipids and other lipid components such as neutral lipids, sterols such as cholesterol and PEGylated lipids. Ionizable cationic lipids are amphiphilic molecules having a lipophilic region containing one or more hydrocarbon groups and a hydrophilic region containing at least one positively charged or ionizable polar head group. Such cationic lipids are ionized at an appropriate pH and can then form a positively charged complex with nucleic acids, making it easier for the nucleic acids to pass through the plasma membrane of the cell and enter the cytoplasm.

The first siRNA therapeutic to be approved, Onpattro (patisiran), entered the market just a few years ago for treatment of hereditary amyloidogenic transthyretin (TTR) amyloidosis. Patisiran's therapeutic effect relies on siRNA-mediated TTR gene silencing, preventing mutant protein production to at least prevent disease progression. The efficient delivery of the siRNA depends upon the LNP technology. Even more recently, nucleic acid vaccines are being used for the treatment and prevention of various diseases, including against the severe acute respiratory syndrome coronavirus 2 (SARS-CoV-2) responsible for causing the on-going worldwide pandemic of the severely infectious coronavirus disease 2019 (COVID-19). mRNA vaccines rely on the delivery of the mRNA into the cytoplasm of host cells, where it is transcribed into antigenic proteins to trigger the production of an immune response. The large size and negative charge of mRNA prevents cellular uptake and so LNPs are again necessary for appropriate delivery.

Methods of preparing LNPs may include solvent mixing, homogenization and microfluidics. However, these methods often produce a heterogeneous mixture that does not always provide for efficient delivery of the mRNA cargo. Accordingly, there is a need for methods that can be used to produce an enriched population of LNP, for example one that is less heterogeneous, which is suitable for use for the delivery of mRNA to a subject.

### SUMMARY

The present disclosure is based, at least in part, on the experimental finding that efficacy of a composition comprising lipid nanoparticles can be improved by removing unencapsulated mRNA from the composition. Accordingly, the present application provides a method of preparing an enriched population of lipid nanoparticles comprising the steps of: contacting a composition comprising lipid nanoparticles and mRNA with an anion exchanger under conditions such that the anion exchanger binds unencapsulated mRNA; and collecting the effluent to obtain the enriched population of lipid nanoparticles. In one example, the unencapsulated mRNA comprises free mRNA, mRNA that is associated with the surface of the lipid nanoparticles and/or partially exposed mRNA.

In one example, the composition comprising lipid nanoparticles has an ionic concentration of between 5 mM and 50 mM. In one example, the composition comprising lipid nanoparticles and RNA comprises salt at a concentration of not greater than 100 mM. In one example, the composition comprising lipid nanoparticles and RNA comprises salt at a concentration of not greater than 40 mM. In one example, the composition comprising lipid nanoparticles has conductivity of less than 15 mS/cm.

In one example, the composition comprising lipid nanoparticles further comprises a buffer selected from citrate buffer, bis-tris, histidine, acetate buffer, phosphate buffer, tris buffer and/or combinations thereof. In one example, the buffer is a bis-tris buffer, histidine buffer or a Tris buffer.

In one example, the buffer is a citrate buffer or a Tris buffer. In one example, the buffer is citrate. In one example, the buffer is Tris. In one example, the composition contains one or more additional components selected from sugars, polymers and detergents.

In one example, the lipid nanoparticle comprises a lipid component and mRNA. In one example, the lipid component comprises a lipid selected from the group consisting of an ionizable lipid, a neutral lipid, a lipid conjugated to a hydrophilic polymer, a structural lipid and combinations thereof. In one example, the lipid component comprises an ionizable lipid, a neutral lipid, a lipid conjugated to a hydrophilic polymer and a structural lipid. In one example, the lipid component comprises an ionizable lipid, a neutral lipid, a PEGylated lipid and a structural lipid.

In one example, the LNP comprises an ionizable lipid and one or more of a neutral lipid, a PEGylated lipid, and a structural lipid.

In one example, the ionisable lipid is selected from the group consisting of:
3-(didodecylamino)-N1,N1,4-tridodecyl-1-piperazineethanamine (KL10),
N1-[2-(didodecylamino)ethyl]-N1,N4,N4-tridodecyl-1,4-piperazinediethanamine (KL22),
14,25-ditridecyl-15,18,21,24-tetraaza-octatriacontane (KL25),
1,2-dilinoleyloxy-N,N-dimethylaminopropane (DLin-DMA),
2,2-dilinoleyl-4-dimethylaminomethyl-[1,3]-dioxolane (DLin-K-DMA),
1,2-dioleoyl-3-trimethylammonium propane (DOTAP),
1,2-distearyloxy-N,N-dimethyl-3-aminopropane (DSDMA),
2,2-dilinoleyl-4-(2-dimethylaminoethyl)-[1,3]-dioxolane (DLin-KC2-DMA),
1,2-dioleyloxy-N,N-dimethylaminopropane (DODMA),
2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA),
(2R)-2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA (2R)),
(2S)-2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA (2S)),
1,2-dilinolenyloxy-N,N-dimethyl-3-aminopropane (DLenDMA),
2,5-bis((9z,12z)-octadeca-9,12,dien-1-yloxyl)benzyl-4-(dimethylamino)butnoate (LKY750),
8-[(2-hydroxyethyl)[6-oxo-6-(undecyloxy)hexyl]amino]-octanoic acid, 1-octylnonyl ester (SM-102),
2-hexyl-decanoic acid, 1,1′-[[(4-hydroxybutyl)imino]di-6,1-hexanediyl]ester (ALC-0315),
4-(dimethylamino)-butanoic acid, (10Z,13Z)-1-(9Z,12Z)-9,12-octadecadien-1-yl-10,13-nonadecadien-1-yl ester (DLin-MC3-DMA or MC3)
((4-hydroxybutyl)azanediyl)bis(hexane-6,1-diyl)bis(2-hexyldecanoate)), and
8-[(2-hydroxyethyl)[6-oxo-6-(undecyloxy)hexyl]amino]-octanoic acid, 1-octylnonyl ester.

In one example, the neutral lipid is selected from the group consisting of 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), and sphingomyelin.

In one example, the PEGylated lipid is selected from the group consisting of PEG-modified phosphatidylethanolamines, PEG-modified phosphatidic acids, PEG-modified ceramides, PEG-modified dialkylamines, PEG-modified diacylglycerols, and PEG-modified dialkylglycerols, optionally PEG-c-DOMG, PEG-DMG, PEG-DLPE, PEG-DMPE, PEG-DPPC, and PEG-DSPE.

In one example, the structural lipid is selected from the group consisting of cholesterol, fecosterol, sitosterol, campesterol, stigmasterol, brassicasterol, ergosterol, tomatidine, tomatine, ursolic acid and alpha-tocopherol. In one example, the structural lipid is cholesterol and/or campesterol.

In one example, the LNP comprises a lipid component comprising: about 25 mol % to about 60 mol % of an ionisable lipid; about 2 mol % to about 25 mol % neutral lipid; about 18.5 mol % to about 60 mol % structural lipid; and about 0.2 mol % to about 10 mol % of PEGylated lipid.

In one example, the mRNA is a self-amplifying mRNA (sa-mRNA) or a conventional mRNA (cRNA). In one example, the mRNA is a self-amplifying mRNA. In one example, the mRNA is cRNA.

In one example, the mRNA is greater than 500 nt in length. In one example, the RNA is between 500 nt and 20,000 nt in length.

In one example, the anion exchanger is an anion exchange resin or an anion exchange membrane. In one example, the anion exchanger is an anion exchange membrane selected from Mustang® Q, Sartobind® Q, Chromasorb®, Capto® Q, Q Sepharose Fast Flow (QSFF), Poros® Q, Fractogel® EMD, Natrix® Q or Eshmuno® Q membrane. In one example, the anion exchanger is a Mustang® Q membrane.

In one example, the method further comprises eluting the unencapsulated mRNA from the anion exchanger.

In one example, the lipid nanoparticle has a diameter of from about 30 nm to about 160 nm. In one example, the lipid nanoparticle has a diameter of from about 60 nm to about 130 nm. In one example, the lipid nanoparticle has a diameter of from about 70 nm to about 120 nm. In one example, the lipid nanoparticle has a diameter of from about 80 nm to about 120 nm. In one example, the lipid nanoparticle has a diameter of from about 70 nm to about 100 nm.

The present disclosure also provides a composition comprising an enriched an enriched population of lipid nanoparticles produced by the method described herein. In one example, least 90% of the RNA is encapsulated within the LNP. In one example, the percentage of RNA encapsulated is measured using anion exchange chromatography. In one example, the percentage RNA encapsulated is measured using the Ribogreen assay. In one example, the percentage of RNA encapsulated is measured using by determining the amount of RNA present in the composition before and after contacting with an anion exchanger.

The present disclosure also provides an enriched lipid nanoparticle composition comprising (i) a plurality of lipid nanoparticles wherein each LNP comprises an ionizable lipid, a neutral lipid, a PEGylated lipid, and a structural lipid; and (ii) mRNA, wherein at least 90% of the mRNA is encapsulated within the LNP. In one example, at least about 95% of the mRNA is encapsulated within the LNP. In one example, at least about 97% of the mRNA is encapsulated within the LNP.

The present disclosure also provides a pharmaceutical composition comprising an enriched an enriched population of lipid nanoparticles produced by the method described herein, and a pharmaceutically acceptable carrier.

The present disclosure also provides a pharmaceutical composition comprising the enriched population of lipid nanoparticles as described herein, and a pharmaceutically acceptable carrier.

In one example, the potency (e.g. in vitro potency) of the composition is at least 2 fold, 4 fold, 6 fold 8 fold or 10 fold greater than an untreated composition, wherein treatment comprises contacting with an anion exchanger. In one example, the potency (e.g. in vitro potency) of the composition is at least 4 fold greater than an untreated composition.

The present disclosure also provides a method of delivering a mRNA to a mammalian cell, including administering the pharmaceutical composition described herein, to a subject to thereby contact the cell with the lipid nanoparticle and deliver the mRNA to the cell. In one example, the cell is a cell of a human subject.

The present disclosure also provides a method of producing a polypeptide of interest in a mammalian cell, including the step of contacting the cell with the pharmaceutical composition described herein.

The present disclosure also provides a method of treating a disease, disorder or condition in a subject in need of such treatment, comprising administering the pharmaceutical composition described herein, to the subject to thereby treat the disease, disorder or condition. In one example, the disease, disorder or condition is selected from the group consisting of a rare disease, an infectious disease, cancer, a proliferative disease, a genetic disease, an autoimmune disease, diabetes, a neurodegenerative disease, a cardiovascular disease, a reno-vascular disease and a metabolic disease.

The present disclosure also provides use of the pharmaceutical composition described herein, in the manufacture of a medicament for the treatment of a disease, disorder or condition. In one example, the disease, disorder or condition is selected from the group consisting of a rare disease, an infectious disease, cancer, a proliferative disease, a genetic disease, an autoimmune disease, diabetes, a neurodegenerative disease, a cardiovascular disease, a reno-vascular disease and a metabolic disease.

The present disclosure also provides a vaccine comprising the composition described herein, the enriched lipid nanoparticle composition described herein, or the pharmaceutical composition described herein. In one example, the vaccine is selected from a tumor vaccine, an influenza vaccine, and a SARS, including a SARS-CoV-2, vaccine.

The present disclosure also provides a method of generating an immune response in a subject, the method comprising administering to the subject a composition comprising an enriched population of LNPs, wherein the LNPs comprise an ionizable lipid, a phospholipid, a PEG lipid, and a structural lipid, wherein at least 90/a of the LNPs comprise RNA encapsulated within the LNP.

In one example, the potency (e.g. in vitro potency) of the composition is at least 1.5-fold, 2-fold, 4-fold, 6-fold, 8-fold or 10-fold greater than an untreated composition, wherein treatment comprises contacting with an anion exchanger. In one example, the potency (e.g. in vitro potency) of the composition is at least 4 fold greater than an untreated composition.

In one example, the amount of RNA administered to a subject is approximately 10%, 20%, 30%, 30% or 50% of the RNA administered to a subject for an untreated composition, wherein treatment comprises contacting with an anion exchanger. In one example, the amount of RNA administered to a subject is approximately 10% of the RNA administered to a subject when the composition has not been filtered using an anion exchange filter.

In one example, the amount of RNA administered to a subject is about 10 μg or less.

### BRIEF DESCRIPTION OF THE DRAWINGS

FIG. 1 illustrates the in vitro activity and potency (the probability of successful transfection per unit of mass of RNA) of treated and untreated LNPs as measured by AF4-MALS and fluorescence-activated cell sorting (FACS).

FIG. 2 illustrates in-vitro expression levels for (A) H5 and (B) N1 for filtered and unfiltered LNPs.

FIG. 3 illustrates the total IgG response as quantified by an ELISA from mice immunized with treated and untreated LNPs on day 21 (A) and day 42 (B) post first vaccination.

FIG. 4 illustrates hemagglutinin titres from mice immunized with treated and untreated LNPs on day 42 post first vaccination.

FIG. 5 illustrates pseudovirus neutralization titres for mice immunized with the treated and untreated LNPs on day 42 post first vaccination.

FIG. 6 illustrates the microneutralization titres from mice immunized with the treated and untreated LNP in short (A) and long (B) form microneutralization assays on day 42 post first vaccination.

FIG. 7 illustrates antibody responses as assessed by ELLA for mice immunized with treated and untreated LNPs on day 42 post first vaccination.

FIG. 8 illustrates a dose comparison for mice immunised with (A) 0.01 μg self-replicating RNA or (B) 0.1 μg self-replicating RNA.

FIG. 9 is a series of graphical representations showing (A) net % HA-specific CD4+ responses; (B) net % NA-specific CD4+ responses; (C) net % HA-specific CD8+ response; (D) net % NA-specific CD8+ response. The cytokines assayed were IFNγ, IL5 and/or IL13, and IL2 and/or TNFα.

FIG. 10 illustrates a dose comparison for mice immunised with 1 μg self-replicating RNA, 0.1 μg self-replicating RNA, 0.01 μg self-replicating RNA or 0.001 μg self-replicating RNA. Graphs show showing (A) net % HA-specific CD4+ responses; (B) net % NA-specific CD4+ responses; (C) net % HA-specific CD8+ response; (D) net % NA-specific CD8+ response.

KEY TO SEQUENCE LISTING

| | |
|---|---|
| SEQ ID NO: 1 | Nucleotide sequence of construct F500.3 |
| SEQ ID NO: 2 | Nucleotide sequence of construct F602 |

While the sequence listing refers to the DNA sequence, it is also understood that disclosure of the present application includes the RNA equivalent thereof as well the complements thereof, unless the context clearly dictates otherwise.

### DETAILED DESCRIPTION

### General

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter.

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific examples described herein, which are intended for the purpose of exemplification only. Functionally-equivalent products, compositions and methods are clearly within the scope of the present disclosure.

Any example of the present disclosure herein shall be taken to apply mutatis mutandis to any other example of the disclosure unless specifically stated otherwise. Stated another way, any specific example of the present disclosure may be combined with any other specific example of the disclosure (except where mutually exclusive).

Any example of the present disclosure disclosing a specific feature or group of features or method or method steps will be taken to provide explicit support for disclaiming the specific feature or group of features or method or method steps.

Unless specifically defined otherwise, all technical and scientific terms used herein shall be taken to have the same meaning as commonly understood by one of ordinary skill in the art (for example, in organic synthetic chemistry, cell culture, molecular genetics, molecular genetics, immunology, immunohistochemistry, protein chemistry, and biochemistry).

Unless otherwise indicated, the recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al. Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The term “and/or”, e.g., “X and/or Y” shall be understood to mean either “X and Y” or “X or Y” and shall be taken to provide explicit support for both meanings or for either meaning.

The terms “from” and “to”, when indicating a range, shall be understood to mean the range is inclusive of the recited lower and upper values. For example, “x is an integer from 0 to 6” shall be understood as including the situation in which x is not present (x is 0), that in which x is 6, as well as each whole number integer value in between, i.e. x is 1, 2, 3, 4, or 5.

As used herein, “about” means the number itself and/or within 10% of the stated number. For instance, with about 5%, this means 5 and/or any number or range within the range of 4.5 to 5.5, e.g., 4.5 to 4.96, 4.81 to 5.35, etc. In one example, about” means the number itself and/or within 5% of the stated number.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

As used herein the term “derived from” shall be taken to indicate that a specified integer may be obtained from a particular source albeit not necessarily directly from that source. Similarly, the term “based on” shall be taken to indicate that a specified integer may be developed or used from a particular source albeit not necessarily directly from that source.

### Selected Definitions

As used herein, the term “chromatography,” refers to any kind of technique which separates the product of interest (e.g., an LNP comprising encapsulated RNA) from contaminants and/or other components in a preparation.

As used herein, the term “flow-through” refers to a product separation technique in which a preparation containing the product of interest is intended to flow-through a material. In one example, the product of interest flows through the material and the undesirable entities bind to the material. In one example, the material is an anion exchanger. As used herein, the term “effluent” refers to the material which doesn't get adsorbed in the anion exchanger, and was eluted along with the mobile phase (for example, water). In some example, effluent and flow through are used interchangeably.

As used herein, the terms “contaminant” or “impurity” are used interchangeably herein, refer to any foreign or objectionable molecule, including a biological macromolecule such as a DNA, an RNA, and one or more additives which may be present in a sample containing the product of interest that is being separated from one or more of the foreign or objectionable molecules. Additionally, such a contaminant may include any reagent which is used in a step which may occur prior to the separation process. In one example, the contaminants may include aggregates of phospholipids (e.g., DSPC) with a structural lipid (e.g., cholesterol). In one example, the impurities include unencapsulated RNA. In one example, the impurities include partially encapsulated RNA. In one example, the methods described herein are intended to selectively remove unencapsulated or exposed RNA from a sample containing a product of interest.

As used herein, the term “substantially pure” when used in the context of an LNP population refers to an LNP population where at least 90% of the LNP contain encapsulated RNA, for example as measured by anion exchange chromatography or Ribogreen assay. In one example, the percentage of encapsulated RNA is measured using the Ribogreen assay. In one example, the percentage of encapsulated RNA is measured using anion exchange chromatography. In one example, a substantially pure LNP population has an encapsulation percentage of about 95%, or about 97%, or about 99%.

The term “polynucleotide” as used herein refers to a polymer containing at least two deoxyribonucleotides or ribonucleotides in either single- or double-stranded form and includes DNA, RNA, and hybrids thereof. DNA may be in the form of antisense molecules, plasmid DNA, cDNA, PCR products, or vectors. RNA may be in the form of small hairpin RNA (shRNA), messenger RNA (mRNA), antisense RNA, miRNA, micRNA, multivalent RNA, dicer substrate RNA or viral RNA (vRNA), and combinations thereof. mRNA includes sa-mRNA and cRNA. Polynucleotides include those containing known nucleotide analogs or modified backbone residues or linkages, which are synthetic, naturally occurring, and non-naturally occurring, and which have similar binding properties as the reference polynucleotide. Examples of such analogs include, without limitation, phosphorothioates, phosphoramidates, methyl phosphonates, chiral-methyl phosphonates, 2′-O-methyl ribonucleotides, and peptide-nucleic acids (PNAs). Unless specifically limited, the term encompasses polynucleotides containing known analogues of natural nucleotides that have similar binding properties as the reference polynucleotide. Unless otherwise indicated, a particular nucleic acid sequence also implicitly encompasses conservatively modified variants thereof (e.g., degenerate codon substitutions), alleles, orthologs, single nucleotide polymorphisms, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem., 260:2605-2608 (1985); Rossolini et al., Mol. Cell. Probes, 8:91-98 (1994)). In one example, the polynucleotide is mRNA.

As used herein, the terms “disease”, “disorder” or “condition” refers to a disruption of or interference with normal function, and is not to be limited to any specific condition, and will include diseases or disorders.

As used herein, a subject “at risk” of developing a disease, disorder or condition may or may not have detectable disease or symptoms of disease, and may or may not have displayed detectable disease or symptoms of disease prior to the treatment according to the present disclosure. “At risk” denotes that a subject has one or more risk factors, which are measurable parameters that correlate with development of the disease or condition, as known in the art and/or described herein.

As used herein, the terms “treating”, “treat” or “treatment” include administering an RNA or composition described herein to thereby reduce or eliminate at least one symptom of a specified disease or condition.

As used herein, the term “preventing”, “prevent” or “prevention” includes providing prophylaxis with respect to occurrence or recurrence of a specified disease or condition in an individual. An individual may be predisposed to or at risk of developing the disease but has not yet been diagnosed with the disease.

As used herein, the phrase “delaying progression of” includes reducing or slowing down the progression of the disease or condition in an individual and/or at least one symptom of a disease or condition.

The term “pharmaceutical composition” relates to a formulation comprising a therapeutically effective agent, preferably together with pharmaceutically acceptable carriers, diluents and/or excipients. Said pharmaceutical composition is useful for treating, preventing, or reducing the severity of a disease or disorder by administration of said pharmaceutical composition to a subject. A pharmaceutical composition is also known in the art as a pharmaceutical formulation.

An “effective amount” refers to at least an amount effective, at dosages and for periods of time necessary, to achieve the desired result. For example, the desired result may be a therapeutic or prophylactic result. In some examples of the present disclosure, the term “effective amount” or “therapeutically effective amount” of a therapeutic mRNA is an amount sufficient to produce the desired effect, such as an increase or inhibition of expression of a target sequence in comparison to the normal expression level detected in the absence of the mRNA. Suitable assays for measuring expression of a target gene or target sequence include, examination of protein or mRNA levels using techniques known to those of skill in the art such as dot blots, northern blots, In situ hybridization, ELISA, immunoprecipitation, enzyme function, fluorescence or luminescence of suitable reporter proteins, as well as phenotypic assays. The effective amount may vary according to the disease or condition to be treated or factor to be altered and also according to the weight, age, racial background, sex, health and/or physical condition and other factors relevant to the mammal being treated. Typically, the effective amount will fall within a relatively broad range (e.g. a “dosage” range) that can be determined through routine trial and experimentation by a medical practitioner. Accordingly, this term is not to be construed to limit the disclosure to a specific quantity, e.g., weight or number of mRNA. An effective amount can be provided in one or more administrations. For example, the effective amount can be administered in a single dose or in a dose repeated once or several times over a treatment period.

A “therapeutically effective amount” is at least the minimum concentration required to effect a measurable improvement of a particular disease or condition. A therapeutically effective amount herein may vary according to factors such as the disease state, age, sex, and weight of the patient, and the ability of the mRNA of the present disclosure to elicit a desired response in the individual. A therapeutically effective amount is also one in which any toxic or detrimental effects of the mRNA are outweighed by the therapeutically beneficial effects.

A “prophylactically effective amount” shall be taken to mean a sufficient quantity of the mRNA of the disclosure to prevent or inhibit or delay the onset of one or more detectable symptoms of a disease or disorder as described herein.

As used herein, the term “subject” shall be taken to mean any animal including humans, for example a mammal. Exemplary subjects include but are not limited to humans and non-human primates. For example, the subject is a human.

As used herein, the term “mammal” includes humans and both domestic animals such as laboratory animals and household pets (e.g., cats, dogs, swine, cattle, sheep, goats, horses, rabbits), and non-domestic animals such as wildlife and the like.

As used herein, the “zeta potential” is the electrokinetic potential of a lipid, e.g., in a lipid nanoparticle composition.

### Anion Exchanger

The present inventors have found that an enhancement of the efficacy of a composition comprising LNP can be achieved by a method including the use of anion exchanger. According to the present disclosure, an enriched population of lipid nanoparticles can be prepared by contacting a composition comprising lipid nanoparticles and mRNA with an anion exchanger under conditions such that the ion exchanger binds unencapsulated (e.g. exposed) mRNA. mRNA that is protected from the environment, for example by encapsulation within a lipid nanoparticle, does not bind to the anion exchanger and remains in the unbound fraction. The unbound fraction is then separated from the anion exchanger to obtain an enriched population of LNP. In one example, the effluent is collected to obtain an enriched population of lipid nanoparticles. In one example, the unencapsulated RNA bound to the ion exchanger is separated from the composition to obtain an enriched population of LNP.

In this context, the term “anion exchanger” is used to cover any means for performing an anion exchange step. For example, the term “anion exchanger” can refer to a matrix or solid support which is positively charged, e.g. having one or more positively charged ligands, such, as quaternary amino groups, attached thereto. Accordingly, the term “anion exchanger” specifically includes, without limitation, anion exchange resins, matrices, absorbers, membranes (including membrane adsorbers) and the like. Anion exchangers are known to the person skilled in the art. Anion exchangers suitable for use in the methods described herein include, without limitation, Mustang® Q, Sartobind® Q, Chromasorb®, Capto® Q, Q Sepharose Fast Flow (QSFF), Poros® Q, Fractogel® EMD (e.g. Fractogel® EMD TMAE, Fraetogel® HMD TAE highcap and Fractogel® EMD DEAE), Natrix® Q, Eshmuno® Q, DEAE cellulose, QAE SEPHADEX™, etc., which are commercially available. In one example, the “anion exchanger” is an anion exchange membrane. In one example, the anion exchange membrane is a Mustang® Q membrane, such as a Mustang® Q filter.

In general, anion exchange membranes have nominal pore sizes of 0.1 to 100 μm. As a reference, Sartobind® Q (Sartorius AG) is a strong anion exchange membrane having a nominal pore size of 3-5 μm and is commercially available in a single or multiple layer format, and Mustang® Q (Pall Corporation) is a strong anion exchange membrane having a nominal pore size of 0.8 μm and is likewise commercially available in a single or multiple layer format. A “nominal” pore size rating describes the ability of the membrane to retain the majority of particulates at 60 to 98% the rated pore size. In one example, the nominal pore size is between about 0.1 and 5 μm. In one example, the nominal pore size is between about 0.1 and 3 μm. In one example, the nominal pore size is between about 0.1 and 1 μm, e.g. 0.8 μm.

In examples where the anion exchanger is a membrane, the membrane can be made from a variety of suitable materials. In one example, the membrane is polyethersulfone (PES) (e.g., from Millipore or PALL Corp.). In one example, the membrane is regenerated cellulose (RC) (e.g., from Sartorius or Pierce). In one example, the anion exchanger is a Q membrane, which is a positively charged membrane and is an anion exchanger with quaternary amines. For example, the Q membrane is functionalized with quaternary ammonium, R—CH₂—N(CH₃)₃. In some examples, the anion exchanger is a D membrane, which is a weak basic anion exchanger functionalized with diethylamine groups, R—CH₂NH⁺(C₂H₅)₂. In one example, the membrane is a weak basic anion exchanger, with diethylamino ethyl (DEAE) cellulose. In one examples, the membrane comprises quaternary amine functional groups. In one example, the membrane is a polyethersulfone (PES)-based membrane with a cross-linked polymeric coating of quaternary amine functional groups (for example, a Mustang Q membrane).

In examples where the anion exchanger is a membrane, the anion exchanger may comprise a single layer of the membrane or comprise two or more layers of the membrane, for example, 2, 3, 4, 6, 8, 10, 12, 14 or 16 or more layers of the membrane. In one example, the anion exchanger contains 4 layers of the membrane. In one example, the anion exchanger contains 16 layers of the membrane.

In examples where the anion exchanger is a membrane, the anion exchanger may comprise a flat sheet, a pleated sheet or a Unipleat® cartridge. In one example, the anion exchanger is a flat sheet. In one example, the anion exchanger is pleated. In one example, the membrane is a Unipleat® cartridge.

In one example, the anion exchanger, e.g., an anion exchange membrane, is housed within a device used for centrifugation; e.g. spin columns, or for vacuum system e.g. vacuum filter holders, or for filtration with pressure e.g. syringe filters, or for chromatography e.g. a column. In one example, the anion exchanger is housed in syringe filter.

In one example, the anion exchanger is housed in a column which may be run on either a standard chromatography system or a custom chromatography system, such as an AKTA™ Explorer (GE Healthcare), equipped with pressure gauges, sensors, and pump plus pump controllers. In this example, the anion exchanger is installed downstream of a pressure gauge. In one example, the pH and conductivity detectors are installed downstream of the anion exchanger. In one example, the system is thoroughly flushed with water and then with equilibration buffer before the installation of the anion exchanger. In one example, the system with the membrane is flushed with equilibration buffer, for example, until the solution pH and conductivity outlet match the equilibration buffer specification (for example, about five membrane volumes) and a stable baseline is observed. In one example, the composition comprising lipid nanoparticles and RNA is buffer exchanged into equilibration buffer prior to contacting with the anion exchanger. In one example, the feed material is loaded by a pump at a suitable pH (i.e. a pH at which the unencapsulated mRNA has a negative charge while the LNP has a neutral or positive charge), and a suitable conductivity. The operation backpressure, and pH and conductivity changes during the operation are recorded. Finally, the membrane effluent containing an enriched population of LNP is collected. In one example, the membrane effluent containing an enriched population of LNP is collected when an ultraviolet (UV) absorbance trace at 280 nm (although other wavelength can be used such as 260 nm or 254 nm) is 0.2 absorbance units over the baseline, the pool collection is stopped once the UV trace at 280 nm is below 0.2 absorbance units, and the samples from the pool in the membrane effluent fraction are assayed for RNA concentration. In one example, the effluent containing an enriched population of LNP is collected without monitoring the absorbance trace. In one example, the anion exchanger is washed with equilibration buffer after the contacting step. The step recovery is typically calculated using the total RNA loaded and the total RNA in the membrane effluent. In one example, the anion exchange membrane is one-time-use. In one example, the anion exchange membrane can be treated with wash buffer (such as a high salt buffer) and/or regeneration buffer and re-used.

In one example, after collecting the effluent, the anion exchanger is contacted with a high salt buffer to elute the compounds (for example, unencapsulated RNA bound to the anion exchanger). In one example, the high salt buffer comprises at least 200 mM salt, 300 mM salt, 400 mM salt, 500 mM salt or 1 M salt. In some examples, the salt is NaCl, but any suitable salt may be used.

In one example, the composition to be subjected to the enrichment method described herein is a lipid nanoparticle composition prepared using a technique known to the person skilled in the art. As would be appreciated by the person skilled in the art, the composition may contain lipid nanoparticles, RNA encapsulated LNP and unencapsulated RNA. As used herein, “unencapsulated RNA” is defined broadly to include free RNA, RNA which is associated with the surface of the LNP and RNA which is partially encapsulated. In other words, RNA is considered unencapsulated if it is fully or partially exposed to the surrounding environment. The composition may also comprise one or more optional components, such as ethanol, buffers, salts etc.

Optionally, the LNP composition has been subjected to at least one purification step prior to contacting with the anion exchanger.

In one example, the LNP composition is desalted prior to contacting with the anion exchanger. In one example, the LNP composition is subjected to a buffer exchange step prior to contacting with the anion exchanger. The pH of the buffer is such that unencapsulated RNA binds to the anion exchanger, while encapsulated RNA is does not substantially bind to the anion exchanger. In one example, the pH of the composition comprising LNP is adjusted to a pH of less than 10. In one example, the pH of the composition comprising LNP is adjusted to a pH of about 6 to about 8. In one example, the pH of the load material is adjusted to about 7 to 8, or about 7.5. In one example, the pH of the composition comprising LNP is adjusted to a pH, for example of about 6 to about 8, the conductivity of the load material is adjusted to ≤about 50 mS/cm, depending on the pH, and the composition comprising LNP is then contacted with the anion exchanger. In one example, the pH of the composition comprising LNP is adjusted to a pH, for example of about 6.5 to about 7.5, the conductivity of the load material is adjusted to ≤about 50 mS/cm, depending on the pH, and the composition comprising LNP is then contacted with the anion exchanger. In one example, the pH of the composition comprising LNP is adjusted to a pH, for example of about 6 to about 8, the ionic concentration of the load material is adjusted to ≤about 50 mS/cm, depending on the pH, and the composition comprising LNP is then contacted with the anion exchanger. In one example, the pH of the composition comprising LNP is adjusted to a pH, for example of about 6.5 to about 7.5, the ionic concentration of the load material is adjusted to ≤about 50 mM, depending on the pH, and the composition comprising LNP is then contacted with the anion exchanger. In one example, the conductivity of the load material is adjusted to ≤about 50 mS/cm, for example ≤about 40 mS/cm, ≤about 30 mS/cm, ≤about 20 mS/cm, or ≤about 10 mS/cm. In one example, the conductivity of the load material is adjusted to ≤about 20 mS/cm or ≤about 10 mS/cm, depending on the pH. In one example, the ionic concentration of the load material is adjusted to ≤about 50 mM, for example ≤about 40 mM, ≤about 30 mM, ≤about 20 mM, or ≤about 10 mM. In one example, the ionic concentration of the load material is adjusted to ≤about 40 mM or about 36 mM, depending on the pH. Because unencapsulated RNA has a negative charge under these conditions it will be electrostatically bound to the positive functional groups of the anion exchanger. This is because the unencapsulated RNA (negative) and membrane (positive) have opposite charge. Without wishing to be bound by theory, since the negative charge of the encapsulated RNA (i.e. RNA contained within the interior of the lipid nanoparticle) will be shielded from the anion exchanger, under pH and conductivity conditions that induce charge with minimal ionic shielding, the encapsulated RNA will not bind to the membrane while the unencapsulated RNA will bind, allowing the encapsulated RNA to “elute” from the matrix or flow through and be recovered in the effluent.

In one example, there is provided a method for purifying a mRNA encapsulated in a LNP from a composition comprising the mRNA encapsulated in a LNP and at least one contaminant, which method comprises the steps of: (a) passing the composition through an anion exchanger, where the contaminant and the anion exchanger have an opposite charge, at operating conditions comprised of a buffer having a pH and a conductivity selected so that the contaminant and the anion exchanger have an opposite charge, which cause the membrane to bind the contaminant, and (b) recovering the purified mRNA encapsulated in a LNP from the effluent.

In one example, there is provided a method for purifying a mRNA encapsulated in a LNP from a composition comprising the mRNA encapsulated in a LNP and at least one contaminant, which method comprises the steps of: (a) passing the composition through an anion exchanger, where the contaminant and the anion exchanger have an opposite charge, at operating conditions comprised of a buffer having a pH of between 7 and 10 and a conductivity of ≤about 100 mS/cm, which cause the membrane to bind the contaminant, and (b) recovering the purified mRNA encapsulated in a LNP from the effluent.

Regarding analytical assays, RNA content may be determined using techniques known to the person skilled in the art, for example, by absorbance at 260 nm using a spectrophotometer or using a fluorescence based assay, such as Ribogreen.

### Buffers

In some examples, the methods provided herein include a variety of buffers including equilibration, loading and wash buffers. The buffers can include a variety of components. In some examples, the buffers include one or more of the following components: Tris, Bis-Tris, Bis-Tris-Propane, Imidazole, Citrate, Methyl Malonic Acid, Acetic Acid, Ethanolamine, Diethanolamine, Triethanolamine (TEA) and Sodium phosphate. Additionally, any buffer can be pH adjusted up or down with the addition of an acid or base, for example acetic acid, citric acid, HEPES, hydrochloric acid, phosphoric acid, sodium hydroxide, TRIS, or other such acidic and basic buffers to reach a suitable pH. Any buffer system can also be conductivity adjusted up or down using purified water, water for injection (WFI), sodium acetate, sodium chloride, potassium phosphate, or other such low and high salt containing buffers to reach a suitable conductivity.

In some examples, equilibration, loading and wash buffers can be of high or low ionic strength. In some examples, equilibration and loading buffers can be of low ionic strength. In some examples, the buffers comprise a salt, for example a chloride salt such as NaCl. In some examples, the salt concentration may be from 0 to 0.3M. In one example, the salt concentration is 0 mM, 10 mM, 25 mM, 50 mM, 100 mM or 150 mM. In one example, the salt concentration is about 100 mM. In one example, the salt concentration is about 0 mM.

In some examples, the equilibration, loading and wash buffers may also include other components, for example, sugars, polymers, or the like. In some examples, the equilibration, loading and wash buffers may also include a sugar. Suitable sugars, include, but are not limited, to disaccharides (e.g., glucose, sucrose or trehalose or a combination thereof). In some examples, the concentration of the sugar in total ranges between 0% w/w and about 30% w/w. For example, the concentration of the sugar ranges between 0% w/w and about 25% w/w (e.g., about 0-25% w/w, 0-20% w/w, 0-15% w/w, 0-10% w/w, about 5% w/w, about 8% w/w, about 10% w/w, about 15% w/w, about 20% w/w, or about 25% w/w).

In some examples, the equilibration, loading and wash buffers may also include a polymer. Suitable polymers include, but are not limited to, poloxamers (Pluronic®), poloxamines (Tetronic®), poly oxy ethylene glycol sorbitan alkyl esters (polysorbates) and polyvinyl pyrrolidones (PVPs). As would be understood by the person skilled in the art, the components of the buffer should not disrupt or should cause minimal disruption of the LNP. In some examples, the polymer is present at a concentration ranging between about 0.1% w/v and about 3% w/v, or between about 0.1% w/w and about 3% w/w. For example, the polymer is present at a concentration ranging between about 0.1% w/v and about 3% w/v, or between about 0.1% w/w and about 3% w/w.

### Lipid Nanoparticles

The methods of the present disclosure can be used to prepare an enriched population of LNP. In some examples, the LNP (for example, the enriched population of LNP) can be used for delivery of an mRNA to a subject. The inventors of the present application have found that the enriched population of LNP have increased potency relative to unfiltered LNP.

As used herein, the term “lipid nanoparticle” or “LNP” shall be understood to refer to lipid-based particles having at least one dimension in the order of nanometers (e.g., 1-1,000 nm). In one example, the term “lipid nanoparticle” includes any lipid based particle, including, but not limited to, liposomes or vesicles, where an aqueous volume is encapsulated by amphipathic lipid bilayers (e.g., single; unilamellar or multiple; multilamellar), micelle-like lipid nanoparticles having a non-aqueous core and solid lipid nanoparticles. In examples, the lipid nanoparticle or LNP may have a structure that includes a single monolayer or bilayer of lipids that encapsulates a solid phase. In one example, the lipid nanoparticle or LNP does not have an aqueous phase or other liquid phase in its interior. In one example, the lipid nanoparticle or LNP does not have a substantial aqueous phase or other liquid phase in its interior. In one example, the LNP is formed by combining an aqueous composition comprising RNA and an organic composition comprising lipids.

In examples, LNPs are formulated in a composition for delivery of an mRNA to a desired target such as a cell, tissue, organ, tumor, and the like.

In one example, the lipid nanoparticle comprises a lipid component and mRNA. In one example, the lipid component comprises a lipid selected from the group consisting of an ionizable lipid, a neutral lipid, a lipid conjugated to a hydrophilic polymer, a structural lipid and combinations thereof. In one example, the lipid component comprises an ionizable lipid, a neutral lipid, a lipid conjugated to a hydrophilic polymer and a structural lipid. In one example, the lipid component comprises an ionizable lipid, a neutral lipid, a PEGylated lipid and a structural lipid.

The LNPs generally comprise an ionizable and/or cationic lipid and one or more of a neutral lipid, charged lipid, sterol and PEGylated lipid.

A “cationic lipid”, “ionizable cationic lipid”, “cationic lipid compound”, “ionizable cationic lipid compound”, or like terms, refer to a lipid compound which is capable of bearing a positive charge. In one example, ionizable cationic lipids disclosed herein include one or more nitrogen-containing groups which may bear the positive charge. These compounds are ionizable such that they can exist in a positively charged or neutral form, depending on pH. The ionization of the cationic lipid affects the surface charge of the lipid nanoparticle under different pH conditions. In one example, the cationic lipid has a positive charge at a pH less than about 7, less than about 6, less than about 5.

The LNP may comprise a cationic and/or ionizable lipid, for example a cationic and/or ionizable lipid comprising a cyclic or non-cyclic amine. Such additional cationic and/or ionizable lipids may be selected from the non-limiting group consisting of:
3-(didodecylamino)-N1,N1,4-tridodecyl-1-piperazineethanamine (KL10),
N1-[2-(didodecylamino)ethyl]-N1,N4,N4-tridodecyl-1,4-piperazinediethanamine (KL22),
14,25-ditridecyl-15,18,21,24-tetraaza-octatriacontane (KL25),
1,2-dilinoleyloxy-N,N-dimethylaminopropane (DLin-DMA),
2,2-dilinoleyl-4-dimethylaminomethyl-[1,3]-dioxolane (DLin-K-DMA),
1,2-dioleoyl-3-trimethylammonium propane (DOTAP),
1,2-distearyloxy-N,N-dimethyl-3-aminopropane (DSDMA),
2,2-dilinoleyl-4-(2-dimethylaminoethyl)-[1,3]-dioxolane (DLin-KC2-DMA),
1,2-dioleyloxy-N,N-dimethylaminopropane (DODMA),
2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA),
(2R)-2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA (2R)),
(2S)-2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA (2S)),
1,2-dilinolenyloxy-N,N-dimethyl-3-aminopropane (DLenDMA),
2,5-bis((9z,12z)-octadeca-9,12,dien-1-yloxyl)benzyl-4-(dimethylamino)butnoate (LKY750),
8-[(2-hydroxyethyl)[6-oxo-6-(undecyloxy)hexyl]amino]-octanoic acid, 1-octylnonyl ester (also referred to as heptadecan-9-yl 8-[2-hydroxyethyl-(6-oxo-6-undecoxyhexyl)amino]octanoate) (SM-102),
2-hexyl-decanoic acid, 1,1′-[[(4-hydroxybutyl)imino]di-6,1-hexanediyl]ester (also referred to as ((4-hydroxybutyl)azanediyl)bis(hexane-6,1-diyl)bis(2-hexyldecanoate)) (ALC-0315),
4-(dimethylamino)-butanoic acid, (10Z,13Z)-1-(9Z,12Z)-9,12-octadecadien-1-yl-10,13-nonadecadien-1-yl ester (DLin-MC3-DMA or MC3)
((4-hydroxybutyl)azanediyl)bis(hexane-6,1-diyl)bis(2-hexyldecanoate)), and
8-[(2-hydroxyethyl)[6-oxo-6-(undecyloxy)hexyl]amino]-octanoic acid, 1-octylnonyl ester.

In one example, the phospholipid is 2,5-bis((9z,12z)-octadeca-9,12,dien-1-yloxyl)benzyl-4-(dimethylamino)butnoate (also referred to as LKY750).

The term “charged lipid” refers to any of a number of lipid species that exist in either a positively charged or negatively charged form independent of the pH within a useful physiological range e.g. pH ~3 to pH ~9. Non-limiting examples of charged lipids include phosphatidylserines, phosphatidic acids, phosphatidylglycerols, phosphatidylmositols, sterol hemisuccinates, dialkyl trimethylammonium-propanes, (including DOTAP and DOTMA), dialkyl dimethylaminopropanes, ethyl phosphocholines, and dimethylaminoethane carbamoyl sterols.

In examples, the LNP additionally comprises one or more of a PEG-lipid, a sterol structural lipid and/or a neutral lipid.

In one example, the present disclosure provides an LNP comprising a neutral lipid. The term “neutral lipid” refers to any of a number of lipid species that exist either in an uncharged or neutral zwitterionic form at a selected pH. Neutral lipids may also be referred to as “zwitterionic lipids”. At physiological PH, such lipids include, but are not limited to, phosphatidylcholines such as 1,2-Distearoyl-sn-glycero-3-phosphocholine (DSPC), 1,2-Dipalmitoyl-sn-glycero-3-phosphocholine (DPPC), 1,2-Dimyristoyl-sn-glycero-3-phosphocholine (DMPC), 1-Palmitoyl-2-oleoyl-sn-glycero-3-phosphocholine (POPC), 1,2-dioleoyl-sn-glycero-3-phosphocholine (DOPC), and phosphatidylethanolamines such as 1,2-Dioleoyl-sn-glycero-3-phosphoethanolamine (DOPE), sphingomyelins (SM). Suitable neutral or zwitterionic lipids for use in the present disclosure will be apparent to the skilled person and include, in examples, 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), and sphingomyelin. The lipids can be saturated or unsaturated. In one example, the neutral lipid is DSPC.

In one example, the present disclosure provides an LNP comprising a structural lipid. Exemplary structural lipids include, but are not limited to, cholesterol, fecosterol, sitosterol, campesterol, stigmasterol, brassicasterol, ergosterol, tomatidine, tomatine, ursolic acid and alpha-tocopherol and mixtures thereof.

In one example, the structural lipid is a sterol. In examples, the structural lipid is cholesterol. In another example, the structural lipid is campesterol.

In one example, the present disclosure provides an LNP comprising a lipid conjugated to a hydrophillic polymer, such as polyethylene glycol (PEG). In one example, the present disclosure provides an LNP comprising a PEGylated lipid. It will be apparent to the skilled person that reference to a PEGylated lipid is a lipid that has been modified with polyethylene glycol. Exemplary PEGylated lipids include, but are not limited to, PEG-modified phosphatidylethanolamines, PEG-modified phosphatidic acids, PEG-modified ceramides, PEG-modified dialkylamines, PEG-modified diacylglycerols, and PEG-modified dialkylglycerols. For example, a PEG lipid includes PEG-c-DOMG, PEG-DMG, PEG-DLPE, PEG-DMPE, PEG-DPPC, a PEG-DSPE lipid and combinations thereof. In one example, the average molecular weight of the PEG is 5000 or less, 4000 or less, 3000 or less, 2000 or less, 1000 or less. In one example, the average molecular weight of the PEG is about 2000. In one example, the PEG lipid comprises DMG-PEG 2000. In one example, the PEGylated lipid is not a hydroxyl-PEG-lipid. In one example, the PEGylated lipid is a methoxy-PEG lipid.

In examples, the LNPs comprise an ionisable and/or cationic lipid; a neutral lipid; a sterol such as cholesterol; and a PEGylated lipid. In some examples, the phospholipid may be DOPE or DSPC. In other examples, the PEG lipid may be PEG-DMG (e.g. DMG-PEG 2000) and/or the structural lipid may be cholesterol. In one example, the LNPs comprise an ionisable and/or cationic lipid; DSPC; cholesterol; and a DMG-PEG2000. In other examples, the cationic and/or ionisable lipid may be LKY750.

The LNPs are formulated with an mRNA to be delivered to a subject.

In some examples, the lipid component of the LNP formulation comprises about mol % to about 60 mol % compound of a cationic and/or ionisable lipid, about 2 mol % to about 25 mol % phospholipid (neutral lipid), about 18.5 mol % to about 60 mol % structural lipid (sterol), and about 0.2 mol % to about 10 mol % of PEGylated lipid, provided that the total mol % does not exceed 100%. In some examples, the lipid component of the LNP formulation comprises about 30 mol % to about 50 mol % compound of cationic and/or ionizable lipid, about 5 mol % to about 20 mol % phospholipid, about 30 mol % to about 55 mol % structural lipid, and about 1 mol % to about 5 mol % of PEGylated lipid. In a particular example, the lipid component includes about 40 mol % cationic and/or ionisable lipid, about 10 mol % phospholipid, about 48 mol % structural lipid, and about 2.0 mol % of PEG lipid.

In examples, the LNPs have a mean diameter of from about 30 nm to about 160 nm, from about 40 nm to about 160 nm, from about 50 nm to about 160 nm, from about 60 nm to about 160 nm, from about 70 nm to about 160 nm, from about 50 nm to about 140 nm, from about 60 nm to about 130 nm, from about 70 nm to about 120 nm, from about 80 nm to about 120 nm, from about 90 nm to about 120 nm, from about 70 to about 110 nm, from about 80 nm to about 110 nm, or about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, 150 nm, 155 nm or 160 nm. In one example, the lipid nanoparticle has a diameter of from about 70 nm to about 130 nm, about 70 nm to about 120 nm, about 70 nm to about 110 nm, about 70 nm to about 100 nm, or about 70 nm to about 90 nm. In one example, the lipid nanoparticle has a diameter of from about 70 nm to about 120 nm. In examples, the LNPs have a mean diameter of from about 80 nm to about 120 nm. In one example, the lipid nanoparticle has a diameter of from about 70 nm to about 100 nm.

The diameter of the LNP may be measured by dynamic light scattering (DLS), transmission electron microscopy (TEM), scanning electron microscopy (SEM), or other methods such as are known in the art.

In some examples, the particle size of the LNPs may be relatively homogenous. A polydispersity index may be used to indicate the homogeneity of the LNPs. A small, for example less than 0.3 or less than 0.2, polydispersity index generally indicates a narrow particle size distribution. A composition of the LNPs described herein may have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some examples, the polydispersity index of the LNP composition may be from about 0 to about 0.20 or 0.05 to 0.20.

Suitable methods for the production of a lipid nanoparticle will be apparent to the skilled person and/or described herein. LNPs comprising an mRNA component and at least one lipid component can be formed, for example, using mixing processes such as microfluidics, including herringbone micromixing, and T-junction mixing of two fluid streams, one of which contains the mRNA, typically in an aqueous solution, and the other of which has the various required lipid components, typically in ethanol.

In one example, the LNPs may be prepared by combining a cationic and/or ionisable lipid, a phospholipid (such as DOPE or DSPC, which may be purchased from commercial sources including Avanti Polar Lipids, Alabaster, AL), a PEGylated lipid (such as 1,2-dimyristoyl-sn-glycerol methoxypoly ethylene glycol, also known as PEG-DMG, which may be purchased from commercial sources including Avanti Polar Lipids, Alabaster, AL), and a structural lipid/sterol (such as cholesterol, which may be purchased from commercial sources including Sigma-Aldrich), at concentrations of, for example, about 50 mM in ethanol. Solutions should be refrigerated during storage at, for example, −20° C. The various lipids may be combined to yield the desired molar ratios and diluted with water and ethanol to a final desired lipid concentration of, for example, between about 5.5 mM and about 25 mM.

An LNP composition comprising a mRNA is prepared (as set out in the examples) by combining the above lipid solution with a solution including the mRNA at, for example, a lipid component to mRNA wt:wt ratio from about 5:1 to about 50:1. The lipid solution may be rapidly injected using a NanoAssemblr microfluidic system at flow rates between about 3 ml/min and about 18 ml/min into the mRNA solution to produce a suspension with a water to ethanol ratio between about 1:1 and about 4:1, or between about 2:1 and about 4:1.

For LNP compositions including a mRNA, solutions of the mRNA at concentrations of 1.0 mg/ml in deionized water may be diluted in 50 mM sodium citrate buffer at a pH between 3 and 6 to form a stock solution.

The method for preparing an LNP described above is thought to induce nano-precipitation and particle formation. Alternative processes including, but not limited to, T-junction and direct injection, may be used to achieve the same nano-precipitation and form lipid nanoparticle compositions that can be used in the methods described herein.

Lipid nanoparticle compositions may be further processed prior to or post use in the methods described herein. Suitable techniques include, but are not limited to, dialysis or tangential flow filtration (TFF) to remove ethanol and/or achieve buffer exchange. For example, formulations may be dialyzed twice against a buffer such as phosphate buffered saline (PBS), pH 7.4, at volumes 200 times that of the primary product using Slide-A-Lyzer cassettes (Thermo Fisher Scientific Inc., Rockford, IL) with a molecular weight cutoff of 10 kD. The first dialysis may be carried out at room temperature for 3 hours. The second dialysis may be carried out overnight at 4 C. In another example, the LNP compositions may be further processed by 10-fold dilution into a first buffer, such as 50 mM citrate buffer at pH 6, and subjected to tangential flow filtration (TFF) using a 300 k molecular weight cut-off membrane (mPES) until concentrated to the original volume. Subsequently, the first buffer may be replaced with a second buffer (for example, a second buffer containing 20 mM Tris buffer at pH 7.5, 80 mM sodium chloride, and 3% sucrose) using diafiltration with a 10-fold volume of the second buffer. The LNP solution may be concentrated to a volume of between 5-10 mL, filtered using a 0.2 micron filter, aliquoted into vials, and frozen, for example, at 1° C./min using a Corning® CoolCell® LX Cell Freezing Container until the samples reach −80° C. Samples may be stored at −80° C. until required.

### Messenger RNA

The methods of the present disclosure may be used to prepare an enriched population of lipid nanoparticles which contain encapsulated mRNA.

As used herein, the term “messenger RNA” (also referred to as mRNA) refers to any polynucleotide which encodes a polypeptide of interest and which is capable of being translated to produce the encoded polypeptide of interest in vitro, in vivo, in situ or ex vivo. The mRNA may or may not be chemically modified. The mRNA of the present disclosure encompasses a non-self-replicating mRNA (also referred to as conventional mRNA (cRNA)), a self-replicating RNA (sa-mRNA). In one example, the mRNA is sa-mRNA. In one example, the mRNA is cRNA.

Typically, cRNA comprises, in order from 5′ to 3′: a 5′cap structure, a 5′-UTR, a nucleotide sequence encoding a polypeptide of interest, a 3′-UTR and a tailing sequence (e.g. a polyadenylation signal or poly-A tail). The cRNA of the present disclosure may further comprise a translation internal ribosome entry site (e.g. Kozak consensus sequence or IRES). In some examples, the cRNA may also comprise a chain terminating nucleotide and/or a stem loop.

As used herein, the term “self-replicating RNA” refers to a construct based on an RNA virus that has been engineered to allow expression of heterologous RNA and proteins. Self-replicating RNA can also be referred to as a replicon. Self-replicating RNA can amplify in host cells leading to expression of the desired gene product in the host cell. For example, the present disclosure provides a monocistronic self-replicating RNA. The sa-mRNA of the present disclosure comprises one or more features of a cRNA, however, sa-mRNA further comprises nucleotide sequences encoding non-structural proteins (NSPs) which enables the sa-mRNA to direct its self-replication. Non-structural proteins include at least one or more genes selected from the group consisting of a viral replicase (or viral polymerase), a viral protease, a viral helicase and other non-structural viral proteins. The skilled person will understand that, in one example, self-replicating RNA can be based on the genomic RNA of RNA viruses. The RNA should be positive (+)-stranded so that it can be directly translated after delivery to a cell without the need for intervening replication steps (e.g., reverse transcription). Translation of the RNA results in the production of non-structural proteins (NSPs) which combine to form a replicase complex (i.e., an RNA-dependent RNA polymerase). The replicase complex is the component of the sa-mRNA which amplifies the original RNA producing both antisense and sense transcripts, resulting in production of multiple daughter RNAs, and subsequently the encoded polypeptide of interest. For example, the self-replicating RNA comprises a viral replicase (or viral polymerase).

For example, the sa-mRNA comprises NSPs derived from (or based on) an alphavirus. Exemplary alphaviruses include, but are not limited to, Venezuelan equine encephalitis virus (VEEV; e.g., Trinidad donkey, TC83CR), Semliki Forest virus (SFV), Sindbis virus (SIN), Ross River virus, Western equine encephalitis virus, Eastern equine encephalitis virus, Chikungunya virus, S.A. AR86 virus, Everglades virus, Mucambo virus, Barmah Forest virus, Middelburg virus, Pixuna virus, O'nyong-nyong virus, Getah virus, Sagiyama virus, Bebaru virus, Mayaro virus, Una virus, Aura virus, Whataroa virus, Banbanki virus, Kyzylagach virus, Highlands J virus, Fort Morgan virus, Ndumu virus, and Buggy Creek virus. The term alphavirus may also include chimeric alphaviruses (e.g., as described by Perri et al, (2003) J. Virol. 77(19): 10394-403) that contain genome sequences from more than one alphavirus. In another example, the self-replicating RNA is derived from or based on a virus other than an alphavirus, for example, a positive-stranded RNA virus. Suitable positive-stranded RNA viruses suitable for use in the present disclosure will be apparent to the skilled person and include, for example, a picornavirus, a flavivirus, a rubivirus, a pestivirus, a hepacivirus, a calicivirus, or a coronavirus.

Typically the sa-mRNA also includes a subgenomic (SG) promoter which, when linked to a nucleotide sequence encoding NSPs and/or a polypeptide of interest, drives the expression of the NSPs and/or polypeptide of interest. The present disclosure provides a self-replicating RNA comprising a nucleotide sequence encoding an antigen operably linked to a SG promoter. SG promoters (also known as ‘junction region’ promoters) suitable for use in the present disclosure will be apparent to the skilled person and/or are described herein. In one example, the SG promoter is derived from or based on an alphavirus SG promoter. For example, the SG promoter is a native alphavirus SG promoter. In one example, the native SG promoter is a minimal SG promoter. For example, the minimal SG promoter is the minimal sequence required for initiation of transcription. In one example, the self-replicating RNA comprises the non-structural proteins of the RNA virus, the 5′ and 3′ untranslated regions (UTRs) and the native subgenomic promoter. In another example, the self-replicating RNA comprises a 5′- and a 3′-end UTR of the RNA virus. In one example, the mRNA is a self-replicating RNA, for example, a monocistronic or bicistronic self-replicating RNA as described in PCT/IB2021/061203.

mRNA useful for formulation with the LNPs may include a first region of linked nucleosides encoding a polypeptide of interest (e.g., a coding region), a first flanking region located at the 5′-terminus of the first region (e.g., a 5′-UTR), a second flanking region located at the 3′-terminus of the first region (e.g., a 3′-UTR), at least one 5′-cap region, and a 3′-stabilizing region. In some examples, a mRNA further includes a poly-A region and/or a Kozak sequence (e.g., in the 5′-UTR). In some cases, mRNA may contain one or more intronic sequences capable of being excised from the mRNA. In some examples, a mRNA may include a 5′ cap structure, a chain terminating nucleotide, a stem loop, a poly A sequence, and/or a polyadenylation signal. Any one of the regions of a mRNA may include one or more alternative components (e.g., an alternative nucleoside). For example, the 3′-stabilizing region may contain an alternative nucleoside such as an L-nucleoside, an inverted thymidine, or a 2′-O-methyl nucleoside and/or the coding region, 5′-UTR, 3′-UTR, or cap region may include an alternative nucleoside such as a 5-substituted uridine (e.g., 5-methoxy uridine), a 1-substituted pseudouridine (e.g., 1-methyl-pseudouridine or 1-ethyl-pseudouridine), and/or a 5-substituted cytidine (e.g., 5-methyl-cytidine).

In some examples, the mRNA may contain one or more intronic sequences capable of being excised from the mRNA.

In some examples, the mRNA is greater than 300 nt in length, for example greater than 500 nt or greater than 1000 nt. In one example, the mRNA is between 500 nt and 20,000 nt in length. In one example, the mRNA is between 500 nt and 10,000 nt in length. In one example, the mRNA is between 10,000 nt and 20,000 nt in length. In one example, the mRNA is between 5,000 nt and 20,000 nt in length. In one example, the mRNA is between 10,000 nt and 15,000 nt in length.

mRNAs may be naturally or non-naturally occurring. mRNAs suitable for use with the present LNPs may include one or more naturally occurring components, including any of the canonical nucleotides A (adenosine), G (guanosine), C (cytosine), U (uridine), or T (thymidine). In one example, all or substantially all of the nucleotides comprising (a) the 5′-UTR, (b) the open reading frame (ORF), (c) the 3′-UTR, (d) the poly A tail, and any combination of (a, b, c, or d above) comprise naturally occurring canonical nucleotides A (adenosine), G (guanosine), C (cytosine), U (uridine), or T (thymidine).

In some examples, mRNAs may include one or more alternative components, as described herein, which impart useful properties including increased stability and/or the lack of a substantial induction of the innate immune response of a cell into which the mRNA is introduced. For example, an alternative mRNA exhibits reduced degradation in a cell into which the mRNA is introduced, relative to a corresponding unaltered mRNA. These alternative species may enhance the efficiency of protein production, intracellular retention of the mRNAs, and/or viability of contacted cells, as well as possess reduced immunogenicity.

mRNAs may include one or more modified (e.g., altered or alternative) nucleobases, nucleosides, nucleotides, or combinations thereof. The mRNAs may include any useful modification or alteration, such as to the nucleobase, the sugar, or the internucleoside linkage (e.g., to a linking phosphate/to a phosphodiester linkage/to the phosphodiester backbone). In some examples, one or more alterations are present in each of the nucleobase, the sugar, and the internucleoside linkage.

mRNAs may or may not be uniformly altered along the entire length of the molecule. For example, one or more or all types of nucleotide (e.g., purine or pyrimidine, or any one or more or all of A, G, U, C) may or may not be uniformly altered in a mRNA, or in a given predetermined sequence region thereof.

Different sugar alterations and/or internucleoside linkages (e.g., backbone structures) may exist at various positions in a mRNA. One of ordinary skill in the art will appreciate that the nucleotide analogs or other alteration(s) may be located at any position(s) of a mRNA such that the function of the mRNA is not substantially decreased. An alteration may also be a 5′- or 3′-terminal alteration. In some examples, the mRNA includes an alteration at the 3′-terminus.

The alternative nucleosides and nucleotides can include an alternative nucleobase. A nucleobase of a mRNA is an organic base such as a purine or pyrimidine or a derivative thereof. A nucleobase may be a canonical base (e.g., adenine, guanine, uracil, thymine, and cytosine). These nucleobases can be altered or wholly replaced to provide mRNA molecules having enhanced properties, e.g., increased stability such as resistance to nucleases. Non-canonical or modified bases may include, for example, one or more substitutions or modifications including but not limited to alkyl, aryl, halo, oxo, hydroxyl, alkyloxy, and/or thio substitutions; one or more fused or open rings; oxidation; and/or reduction.

Alternative nucleotide base pairing encompasses not only the standard adenine-thymine, adenine-uracil, or guanine-cytosine base pairs, but also base pairs formed between nucleotides and/or alternative nucleotides including non-standard or alternative bases, wherein the arrangement of hydrogen bond donors and hydrogen bond acceptors permits hydrogen bonding between a non-standard base and a standard base or between two complementary non-standard base structures. One example of such non-standard base pairing is the base pairing between the alternative nucleotide inosine and adenine, cytosine, or uracil.

In some examples, the nucleobase is an alternative uracil. Exemplary nucleobases and nucleosides having an alternative uracil include pseudouridine (ψ), pyridin-4-one ribonucleoside, 5-aza-uracil, 6-aza-uracil, 2-thio-5-aza-uracil, 2-thio-uracil (s2U), 4-thio-uracil (s4U), 4-thio-pseudouridine, 2-thio-pseudouridine, 5-hydroxy-uracil (ho5U), 5-aminoallyl-uracil, 5-halo-uracil (e.g., 5-iodo-uracil or 5-bromo-uracil), 3-methyl-uracil (m3U), 5-methoxy-uracil (mo5U), uracil 5-oxyacetic acid (cmo5U), uracil 5-oxyacetic acid methyl ester (mcmo5U), 5-carboxymethyl-uracil (cm5U), 1-carboxymethyl-pseudouridine, 5-carboxyhydroxymethyl-uracil (chm5U), 5-carboxyhydroxymethyl-uracil methyl ester (mchm5U), 5-methoxycarbonylmethyl-uracil (mcm5U), 5-methoxycarbonylmethyl-2-thio-uracil (mcm5s2U), 5-aminomethyl-2-thio-uracil (nm5s2U), 5-methylaminomethyl-uracil (mnm5U), 5-methylaminomethyl-2-thio-uracil (mnm5s2U), 5-methylaminomethyl-2-seleno-uracil (mnm5se2U), 5-carbamoylmethyl-uracil (ncm5U), 5-carboxymethylaminomethyl-uracil (cmnm5U), 5-carboxymethylaminomethyl-2-thio-uracil (cmnm5s2U), 5-propynyl-uracil, 1-propynyl-pseudouracil, 5-taurinomethyl-uracil (τm5U), 1-taurinomethyl-pseudouridine, 5-taurinomethyl-2-thio-uracil (τm5s2U), 1-taurinomethyl-4-thio-pseudouridine, 5-methyl-uracil (m5U, i.e., having the nucleobase deoxythymine), 1-methyl-pseudouridine (m ψ), 1-ethyl-pseudouridine (Et1ψ), 5-methyl-2-thio-uracil (m5s2U), 1-methyl-4-thio-pseudouridine (m1s4ψ), 4-thio-1-methyl-pseudouridine, 3-methyl-pseudouridine (m3ψ), 2-thio-1-methyl-pseudouridine, 1-methyl-1-deaza-pseudouridine, 2-thio-1-methyl-1-deaza-pseudouridine, dihydrouracil (D), dihydropseudouridine, 5,6-dihydrouracil, 5-methyl-dihydrouracil (m5D), 2-thio-dihydrouracil, 2-thio-dihydropseudouridine, 2-methoxy-uracil, 2-methoxy-4-thio-uracil, 4-methoxy-pseudouridine, 4-methoxy-2-thio-pseudouridine, N1-methyl-pseudouridine, 3-(3-amino-3-carboxypropyl)uracil (acp3U), 1-methyl-3-(3-amino-3-carboxypropyl)pseudouridine (acp3 ψ), 5-(isopentenylaminomethyl)uracil (inm5U), 5-(isopentenylaminomethyl)-2-thio-uracil (inm5s2U), 5,2′-O-dimethyl-uridine (m5Um), 2-thio-2′-O_methyl-uridine (s2Um), 5-methoxycarbonylmethyl-2′-O-methyl-uridine (mcm5Um), 5-carbamoylmethyl-2′-O-methyl-uridine (ncm5Um), 5-carboxymethylaminomethyl-2′-O-methyl-uridine (cmnm5Um), 3,2′-O-dimethyl-uridine (m3Um), and 5-(isopentenylaminomethyl)-2′-O-methyl-uridine (inm5Um), 1-thio-uracil, deoxythymidine, 5-(2-carbomethoxyvinyl)-uracil, 5-(carbamoylhydroxymethyl)-uracil, 5-carbamoylmethyl-2-thio-uracil, 5-carboxymethyl-2-thio-uracil, 5-cyanomethyl-uracil, 5-methoxy-2-thio-uracil, and 5-[3-(1-E-propenylamino)]uracil. In one example, the modified uracil is pseudouridine. In one example, the modified uracil is N1-methyl-pseudouridine.

In some examples, the nucleobase is an alternative cytosine. Exemplary nucleobases and nucleosides having an alternative cytosine include 5-aza-cytosine, 6-aza-cytosine, pseudoisocytidine, 3-methyl-cytosine (m3C), N4-acetyl-cytosine (ac4C), 5-formyl-cytosine (f5C), N4-methyl-cytosine (m4C), 5-methyl-cytosine (m5C), 5-halo-cytosine (e.g., 5-iodo-cytosine), 5-hydroxymethyl-cytosine (hm5C), 1-methyl-pseudoisocytidine, pyrrolo-cytosine, pyrrolo-pseudoisocytidine, 2-thio-cytosine (s2C), 2-thio-5-methyl-cytosine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytosine, 2-methoxy-5-methyl-cytosine, 4-methoxy-pseudoisocytidine, 4-methoxy-1-methyl-pseudoisocytidine, lysidine (k2C), 5,2′-O-dimethyl-cytidine (m5Cm), N4-acetyl-2′-O-methyl-cytidine (ac4Cm), N4,2′-O-dimethyl-cytidine (m4Cm), 5-formyl-2′-O-methyl-cytidine (f5Cm), N4,N4,2′-O-trimethyl-cytidine (m42Cm), 1-thio-cytosine, 5-hydroxy-cytosine, 5-(3-azidopropyl)-cytosine, and 5-(2-azidoethyl)-cytosine. In one example, the modified cytosine is 5-methyl-cytosine.

In some examples, the nucleobase is an alternative adenine. Exemplary nucleobases and nucleosides having an alternative adenine include 2-amino-purine, 2,6-diaminopurine, 2-amino-6-halo-purine (e.g., 2-amino-6-chloro-purine), 6-halo-purine (e.g., 6-chloro-purine), 2-amino-6-methyl-purine, 8-azido-adenine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenine (m1A), 2-methyl-adenine (m2A), N6-methyl-adenine (m6A), 2-methylthio-N6-methyl-adenine (ms2m6A), N6-isopentenyl-adenine (i6A), 2-methylthio-N6-isopentenyl-adenine (ms2i6A), N6-(cis-hydroxyisopentenyl)adenine (io6A), 2-methylthio-N6-(cis-hydroxyisopentenyl)adenine (ms2io6A), N6-glycinylcarbamoyl-adenine (g6A), N6-threonylcarbamoyl-adenine (t6A), N6-methyl-N6-threonylcarbamoyl-adenine (m6t6A), 2-methylthio-N6-threonylcarbamoyl-adenine (ms2g6A), N6,N6-dimethyl-adenine (m62A), N6-hydroxynorvalylcarbamoyl-adenine (hn6A), 2-methylthio-N6-hydroxynorvalylcarbamoyl-adenine (ms2hn6A), N6-acetyl-adenine (ac6A), 7-methyl-adenine, 2-methylthio-adenine, 2-methoxy-adenine, N6,2′-O-dimethyl-adenosine (m6Am), N6,N6,2′-O-trimethyl-adenosine (m62Am), 1,2′-O-dimethyl-adenosine (m1Am), 2-amino-N6-methyl-purine, 1-thio-adenine, 8-azido-adenine, N6-(19-amino-pentaoxanonadecyl)-adenine, 2,8-dimethyl-adenine, N6-formyl-adenine, and N6-hydroxymethyl-adenine.

In some examples, the nucleobase is an alternative guanine. Exemplary nucleobases and nucleosides having an alternative guanine include inosine (I), 1-methyl-inosine (mll), wyosine (imG), methylwyosine (mimG), 4-demethyl-wyosine (imG-14), isowyosine (imG2), wybutosine (yW), peroxywybutosine (o2yW), hydroxywybutosine (OHyW), undermodified hydroxywybutosine (OHyW®), 7-deaza-guanine, queuosine (Q), epoxyqueuosine (oQ), galactosyl-queuosine (galQ), mannosyl-queuosine (manQ), 7-cyano-7-deaza-guanine (preQ0), 7-aminomethyl-7-deaza-guanine (preQ1), archaeosine (G+), 7-deaza-8-aza-guanine, 6-thio-guanine, 6-thio-7-deaza-guanine, 6-thio-7-deaza-8-aza-guanine, 7-methyl-guanine (m7G), 6-thio-7-methyl-guanine, 7-methyl-inosine, 6-methoxy-guanine, 1-methyl-guanine (m1G), N2-methyl-guanine (m2G), N2,N2-dimethyl-guanine (m22G), N2,7-dimethyl-guanine (m2,7G), N2, N2,7-dimethyl-guanine (m2,2,7G), 8-oxo-guanine, 7-methyl-8-oxo-guanine, 1-methyl-6-thio-guanine, N2-methyl-6-thio-guanine, N2,N2-dimethyl-6-thio-guanine, N2-methyl-2′-O-methyl-guanosine (m2Gm), N2,N2-dimethyl-2′-O-methyl-guanosine (m22Gm), 1-methyl-2′-O-methyl-guanosine (m1Gm), N2,7-dimethyl-2′-O-methyl-guanosine (m2,7Gm), 2′-O-methyl-inosine (Im), 1,2′-O-dimethyl-inosine (mllm), 1-thio-guanine, and O-6-methyl-guanine.

The alternative nucleobase of a nucleotide can be independently a purine, a pyrimidine, a purine or pyrimidine analog. For example, the nucleobase can be an alternative to adenine, cytosine, guanine, uracil, or hypoxanthine. In another example, the nucleobase can also include, for example, naturally-occurring and synthetic derivatives of a base, including pyrazolo[3,4-d]pyrimidines, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-propynyl uracil and cytosine, 6-azo uracil, cytosine and thymine, 5-uracil (pseudouracil), 4-thiouracil, 8-halo (e.g., 8-bromo), 8-amino, 8-thiol, 8-thioalkyl, 8-hydroxy and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, deazaguanine, 7-deazaguanine, 3-deazaguanine, deazaadenine, 7-deazaadenine, 3-deazaadenine, pyrazolo[3,4-d]pyrimidine, imidazo[1,5-a]1,3,5 triazinones, 9-deazapurines, imidazo[4,5-d]pyrazines, thiazolo[4,5-d]pyrimidines, pyrazin-2-ones, 1,2,4-triazine, pyridazine; or 1,3,5 triazine.

The enriched population of LNP prepared using the methods described herein may, in some examples, be used to induce expression of a desired protein both in vitro and in vivo by contacting cells with the enriched population of LNP, wherein the LNP encapsulates a mRNA that is expressed to produce the desired protein, such as a mRNA encoding the desired protein.

The mRNA of the present disclosure typically comprises a nucleotide sequence encoding a polypeptide of interest. The nucleotide sequence may encode any polypeptide known to the person skilled in the art, including any naturally or non-naturally occurring or otherwise modified polypeptide. A polypeptide encoded by an mRNA may be of any size and may have any secondary structure or activity. In some examples, a polypeptide encoded by an mRNA may have a therapeutic effect when expressed in a cell. In one example, the nucleotide sequence encodes an antigen e.g., a pathogenic antigen). For example, the antigen can induce an immune response in the subject. In one example, the mRNA of the present disclosure comprises a nucleotide sequence that encodes an antigen from a virus. In one example, the mRNA of the present disclosure comprises a nucleotide sequence that encodes an antigen from a respiratory virus, for example, influenza virus, coronavirus, respiratory syncytial virus (RSV). In one example, the mRNA comprises a nucleotide sequence encoding an antigen as described herein.

mRNAs for formulation with LNPs may be prepared according to any available technique known in the art. mRNA may be prepared by, for example, enzymatic synthesis which provides a process of template-directed synthesis of RNA molecules from an engineered DNA template comprised of an upstream bacteriophage promoter sequence linked to a downstream sequence encoding the gene of interest. Template DNA can be prepared for in vitro transcription from a number of sources with appropriate techniques which are well-known in the art including, but not limited to, plasmid DNA and polymerase chain reaction amplification (see Linpinsel, J. L and Conn, G. L., General protocols for preparation of plasmid DNA template and Bowman, J. C., Azizi, B., Lenz, T. K., Ray, P., and Williams, L. D. in RNA in vitro transcription and RNA purification by denaturing PAGE in Recombinant and in vitro RNA syntheses Methods v. 941 Conn G. L. (ed), New York, N.Y. Humana Press, 2012).

Transcription of the RNA occurs in vitro using the linearized DNA template in the presence of the corresponding RNA polymerase and adenosine, guanosine, uridine and cytidine ribonucleoside triphosphates (rNTPs) under conditions that support polymerase activity while minimizing potential degradation of the resultant mRNA transcripts. In vitro transcription can be performed using a variety of commercially available kits including, but not limited to RiboMax Large Scale RNA Production System (Promega), MegaScript Transcription kits (Life Technologies) as well as with commercially available reagents including RNA polymerases and rNTPs. The methodology for in vitro transcription of mRNA is well-known in the art. (see, e.g. Losick, R., 1972, In Vitro Transcription, Ann Rev Biochem v.41 409-46; Kamakaka, R. T. and Kraus, W. L. 2001. In Vitro Transcription. Current Protocols in Cell Biology. 2:11.6:11.6.1-11.6.17; Beckert, B. And Masquida, B., (2010) Synthesis of RNA by In Vitro Transcription in RNA in Methods in Molecular Biology v. 703 (Neilson, H. Ed), New York, N.Y. Humana Press, 2010; Brunelle, J. L. and Green, R., 2013, Chapter Five—In vitro transcription from plasmid or PCR-amplified DNA, Methods in Enzymology v. 530, 101-114; all of which are incorporated herein by reference). Following In Vitro transcription, the DNA template may be removed, for example, by DNase digestion. The skilled person will understand that synthetic mRNA capping is performed to correct mRNA processing and contribute to stabilization of the mRNA. In one example, the mRNA is enzymatically 5′-capped. In one example, the mRNA is co-transcriptionally capped. For example, the 5′ cap is a cap0 structure or a cap1 structure. In one example, the 5′ cap is a cap0 structure, for example, the 5′-cap (i.e., cap0) consists of an inverted 7-methylguanosine connected to the rest of the mRNA via a 5′-5′ triphosphate bridge. In one example, the 5′ cap is a cap1 structure, for example, the 5′-cap (i.e., cap1) consists of the cap0 with an additional methylation of the 2′O position of the initiating nucleotide.

In one example, the desired in vitro transcribed mRNA is then purified from the undesired components of the transcription or associated reactions. Techniques for the isolation of the mRNA transcripts are well known in the art and include phenol/chloroform extraction, precipitation with either alcohol in the presence of monovalent cations or lithium chloride or chromatography. In another example, the mRNA is purified using tangential flow filtration (TFF). Following purification, the mRNA is resuspended in e.g., nuclease-free water.

### Encapsulation Percentage

Methods for producing LNPs used by the person skilled in the art can produce a mixed or heterogeneous population of LNP. The mixed or heterogeneous population of LNP may include RNA that is partially encapsulated within the LNP, RNA that is associated with surface of the LNP and/or RNA that is not associated with the LNP. An “enriched” LNP population or preparation refers to a LNP population derived from a starting LNP population (e.g., a heterogeneous LNP population such as that prepared by nano-precipitation and the like) that contains a greater percentage of LNP encapsulated RNA than the percentage of LNP encapsulated RNA in the starting population. For example, a starting LNP population can be enriched for an LNP containing fully encapsulated RNA. As used herein, the terms “LNP population” and “LNP preparation” are used interchangeably.

As used herein, “encapsulation percentage” of a population refers to the amount of a RNA that is fully encapsulated within an LNP, relative to the total amount of RNA present in the LNP population. As used herein, “fully encapsulated” refers to complete enclosure, confinement, surrounding, or encasement. For example, if 92 mg of RNA present in the LNP population is fully encapsulated within an LNP out of a total 100 mg of RNA present in the population, the encapsulation efficiency may be given as 92%.

The encapsulation percentage of an LNP population prior to use in the methods of the present disclosure may be at least 10%, for example about 10%, 15%, 20%, 25%, 20%, 35%, 40%, 45%, 50%, 55%, 60%, 65%, 70%, 75%, 80%, 85%, 90%, 91%, 92%, 93%, 94%, 95%, 96%, 97%, 98%, or 99%. In some examples, the encapsulation percentage prior to use in the methods described herein may be at least 10%. In certain examples, the encapsulation percentage may be at least 20%. The encapsulation percentage of an LNP population prior to use in the methods of the present disclosure may be at less than 10%, for example less than about 10%, 15%, 20%, 25%, 20%, 35%, 40%, 45%, 50%, 55%, 60%, 65%, 70%, 75%, 80%, 85%, 90%, 91%, 92%, 93%, 94%, 95%, 96%, 97%, 98%, or 99%. In some examples, the encapsulation percentage prior to use in the methods described herein may be less than 10%. In certain examples, the encapsulation percentage may be less than 20%. In certain examples, the encapsulation percentage may be less than 50%.

As would be understood, the encapsulation percentage of enriched population of LNP will be higher than the encapsulation percentage of an unenriched population (i.e. a population of LNP prior to use in the methods of the present disclosure). The encapsulation percentage of enriched population of LNP (for example, produced by the methods of the present disclosure) may be at least 10%, for example about 10%, 15%, 20%, 25%, 20%, 35%, 40%, 45%, 50%, 55%, 60%, 65%, 70%, 75%, 80%, 85%, 90%, 91%, 92%, 93%, 94%, 95%, 96%, 97%, 98%, or 99%. In some examples, the encapsulation percentage may be at least 80%. In certain examples, the encapsulation percentage may be at least 90%. In certain examples, the encapsulation percentage may be at least 95%.

The amount of RNA present in an LNP population may be determined using techniques known to the person skilled in the art. For example, the amount of RNA contained in an LNP population may be determined using a fluorescence assay employing a dye that becomes more emissive upon binding RNA, such as Ribogreen, or by measuring absorbance at 260 nm. Using the Ribogreen assay as an example, the total amount of RNA is determined by disrupting the LNP with a detergent to expose the encapsulated RNA, adding the dye, and comparing the emission intensity against a standard curve prepared using ribosomal RNA. It was previously thought that the amount of unencapsulated RNA could be estimated in a similar manner if the detergent disruption of the LNP is omitted from the assay. However, the present inventors have found that this underestimates the amount of unencapsulated mRNA and an alternative assay is required to estimate the encapsulated mRNA in a formulation or population of LNP.

In one example, the encapsulation percentage can be determined by comparing the total amount of RNA in a composition before and after contacting with an anion exchanger. In one example, the encapsulation percentage can then be determined using the following formula:

$$Percent Encapsulation ( \% ) = ( {RNA}_{UNBOUND} ) / {RNA}_{LOAD} ) \times 100$$

where RNA_{LOAD} and RNA_{UNBOUND} are, respectively, the absolute amount of RNA in the unbound fraction and loaded onto the anion exchange column.

### Encapsulation Efficiency

As used herein, “encapsulation efficiency” refers to the amount of an mRNA that becomes part of an LNP composition, relative to the initial total amount of mRNA used to prepare the LNP composition. For example, if the LNP formulation contains 92 mg of mRNA and 100 mg of mRNA was initially provided to form the composition, the encapsulation efficiency may be given as 92%. This differs from encapsulation percentage which refers to the amount of mRNA completely encapsulated within LNP in a formulation relative to the total amount of mRNA present in the formulation.

### Lipid Nanoparticle Compositions

The present disclosure provides a composition comprising an enriched population of LNP prepared by the methods described herein.

The present disclosure also provides a pharmaceutical composition comprising an enriched population of lipid nanoparticles prepared by the methods described herein and a pharmaceutically acceptable carrier.

A composition comprising an enriched population of lipid nanoparticles prepared by the methods described herein may be formulated for administration via any accepted mode of administration of lipid particles. The pharmaceutical compositions described herein may be formulated into preparations in solid, semi-solid, liquid or gaseous forms, such as tablets, capsules, powders, granules, ointments, solutions, suspensions, suppositories, injections, inhalants, gels, microspheres, and aerosols. Typical routes of administering such pharmaceutical LNP compositions include, without limitation, oral, topical, transdermal, inhalation, parenteral, sublingual, buccal, rectal, vaginal, and intranasal. The term parenteral as used herein includes subcutaneous injections, intravenous, intramuscular, intradermal, intrasternal injection or infusion techniques. In one example, the LNP is administered parenterally, such as intramuscularly, subcutaneously or intravenously. In some examples, the LNP is administered intramuscularly.

The compositions administered to a subject may be in the form of one or more dosage units, where for example, a tablet or injectable liquid volume may be a single dosage unit. Actual methods of preparing such dosage forms are known, or will be apparent, to those skilled in this art; for example, see Remington: The Science and Practice of Pharmacy, 20th Edition (Philadelphia College of Pharmacy and Science, 2000).

Therefore, in one example there is provided a composition (such as a pharmaceutical composition) comprising an enriched population of lipid nanoparticles, combined with a pharmaceutically acceptable carrier. The composition may optionally comprise pharmaceutically acceptable excipients. “Pharmaceutically acceptable carrier, diluent or excipient”, or like terms, refers to any ingredient other than the compounds described herein (for example, a vehicle capable of suspending, complexing, or dissolving the active compound) and having the properties of being substantially nontoxic and non-inflammatory in a patient.

In general terms, by “carrier” is meant a solid or liquid filler, binder, diluent, encapsulating substance, emulsifier, wetting agent, solvent, suspending agent, coating or lubricant that may be safely administered to any subject, e.g., a human. Depending upon the particular route of administration, a variety of acceptable carriers, known in the art may be used, as for example described in Remington's Pharmaceutical Sciences (Mack Publishing Co. N.J. USA, 1991).

Excipients may include, for example: anti-adherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, and waters of hydration. Exemplary excipients include, but are not limited to: butylated hydroxy toluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E (alpha-tocopherol), vitamin C, xylitol, and other species disclosed herein.

Formulation of LNPs to be administered will vary according to the route of administration and formulation (e.g., solution, emulsion, capsule) selected. An appropriate pharmaceutical composition comprising an LNP to be administered can be prepared in a pharmaceutically acceptable carrier. For solutions or emulsions, suitable carriers include, for example, aqueous or alcoholic/aqueous solutions, emulsions or suspensions, including saline and buffered media. Parenteral vehicles can include sodium chloride solution, Ringer's dextrose, dextrose and sodium chloride, lactated Ringer's or fixed oils. A variety of appropriate aqueous carriers are known to the skilled artisan, including water, buffered water, buffered saline, polyols (e.g., glycerol, propylene glycol, liquid polyethylene glycol), dextrose solution and glycine. Intravenous vehicles can include various additives, preservatives, or fluid, nutrient or electrolyte replenishers (See, generally, Remington's Pharmaceutical Science, 16th Edition, Mack, Ed. 1980). The compositions can optionally contain pharmaceutically acceptable auxiliary substances as required to approximate physiological conditions such as pH adjusting and buffering agents and toxicity adjusting agents, for example, sodium acetate, sodium chloride, potassium chloride, calcium chloride and sodium lactate. The LNPs can be stored in the liquid stage or can be lyophilized for storage and reconstituted in a suitable carrier prior to use according to art-known lyophilization and reconstitution techniques.

When the LNP composition is a vaccine composition then the carrier may be water, typically pyrogen-free water; isotonic saline or buffered (aqueous) solutions, e.g. phosphate, citrate etc. buffered solutions. For injection of an LNP vaccine composition, water or preferably a buffer, more preferably an aqueous buffer, may be used, containing a sodium salt, preferably at least 50 mM of a sodium salt, a calcium salt, preferably at least 0.01 mM of a calcium salt, and optionally a potassium salt, such as at least 3 mM of a potassium salt. In an example, the sodium, calcium and, optionally, potassium salts may be present as their chlorides, iodides, or bromides, or in the form of their hydroxides, carbonates, hydrogen carbonates, or sulfates, etc. Non-limiting examples of sodium salts include e.g. NaCl, NaI, NaBr, Na₂CO₃, NaHCO₃, Na₂SO₄, examples of the optional potassium salts include e.g. KCl, KI, KBr, K₂CO₃, KHCO₃, K₂SO₄, and examples of calcium salts include e.g. CaCl₂, CaI₂, CaBr₂, CaCO₃, CaSO₄, Ca(OH)₂. Furthermore, organic anions of the aforementioned cations may be contained in the buffer. In certain examples, the buffer suitable for injection purposes, may contain salts selected from sodium chloride (NaCl), calcium chloride (CaCl₂) and optionally potassium chloride (KCl), wherein further anions may be present additional to the chlorides. In examples, the salts in the injection buffer are present in a concentration of at least 50 mM sodium chloride (NaCl), at least 3 mM potassium chloride (KCl) and at least 0.01 mM calcium chloride (CaCl₂)). The injection buffer may be hypertonic, isotonic or hypotonic with reference to the specific reference medium.

In one example, the pharmaceutical composition of the disclosure comprises a nanoparticle composition disclosed herein and a pharmaceutically acceptable carrier selected from one or more of Tris, an acetate (e.g., sodium acetate), an citrate (e.g., sodium citrate), saline, PBS, and sucrose. In certain examples, the pharmaceutical composition of the disclosure has a pH value between about 7 and 8 (e.g., 6.8, 6.9, 7.0, 7.1, 7.2, 7.3, 7.4, 7.5, 7.6, 7.7, 7.8, 7.9 or 8.0, or between 7.5 and 8 or between 7 and 7.8).

For example, a pharmaceutical composition of the disclosure comprises a nanoparticle composition disclosed herein, Tris, saline and sucrose, and has a pH of about 7.5-8, which is suitable for storage and/or shipment at, for example, about −20° C. For example, a pharmaceutical composition of the disclosure comprises a nanoparticle composition disclosed herein and PBS and has a pH of about 7-7.8, suitable for storage and/or shipment at, for example, about 4° C. or lower. “Stability,” “stabilized,” and “stable” in the context of the present disclosure refers to the resistance of nanoparticle compositions and/or pharmaceutical compositions disclosed herein to chemical or physical changes (e.g., degradation, particle size change, aggregation, change in encapsulation, etc.) under given manufacturing, preparation, transportation, storage and/or in-use conditions, e.g., when stress is applied such as shear force, freeze/thaw stress, etc.

When the LNP composition is a vaccine composition it may further comprise one or more pharmaceutically acceptable adjuvants to enhance the immunostimulatory properties of the composition. The adjuvant may be any compound, which is suitable to support administration and delivery of the LNP composition and which may initiate or increase an immune response of the innate immune system, i.e. a non-specific immune response. Such an adjuvant may be selected from any adjuvant known to a skilled person and suitable for the particular nature of the vaccine, i.e. for induction of a suitable immune response in a mammal.

In one example, a pharmaceutical composition as described herein may comprise a total lipid content of about 0.1 mg to 10 mg, or 0.5 mg to 8 mg, or 0.7 mg to 6 mg, or 0.7 mg to 2 mg. In some examples, such an immunogenic composition may comprise a total lipid content of about 1 mg/mL-15 mg/mL or 2 mg/mL-10 mg/mL or 2.5-5 mg/mL.

The pharmaceutical compositions described herein may be provided as a frozen concentrate for solution for injection. In one example, for preparation of solution for injection, a frozen concentrate is thawed and diluted with isotonic solution (e.g., 0.9% NaCl, saline), e.g., by a one-step dilution process. In some examples, bacteriostatic sodium chloride solution (e.g., 0.9% NaCl, saline) cannot be used as a diluent. In some examples, the diluted composition is an off-white suspension. The concentration of the final solution for injection varies depending on the respective dose level to be administered.

Compositions described herein may be shipped and/or stored under temperature-controlled conditions, e.g., temperature conditions of about 4-5° C. or below, about −20° C. or below, −70° C.±10° C. (e.g., −80° C. to −60° C.), e.g., utilizing a cooling system (e.g., that may be or include dry ice) to maintain the desired temperature. In one example, compositions described herein are shipped in temperature-controlled thermal shippers.

Such shippers may contain a GPS-enabled thermal sensor to track the location and temperature of each shipment. The compositions can be stored by refilling with, e.g., dry ice.

### Dosage

Upon formulation, compositions of the present disclosure will be administered in a manner compatible with the dosage formulation and in such amount as is therapeutically/prophylactically effective. The dosage ranges for the administration of the enriched population of LNP of the disclosure or compositions thereof are those large enough to produce the desired effect. For example, the composition comprises an effective amount of the mRNA. In one example, the composition comprises a therapeutically effective amount of the mRNA. In another example, the composition comprises a prophylactically effective amount of the mRNA.

The dosage should not be so large as to cause adverse side effects. Generally, the dosage will vary with the age, condition, sex and extent of the disease in the patient and can be determined by one of skill in the art. The dosage can be adjusted by the individual physician in the event of any complication.

Dosage can vary from about 0.1 mg/kg to about 300 mg/kg, e.g., from about 0.2 mg/kg to about 200 mg/kg, such as, from about 0.5 mg/kg to about 20 mg/kg, in one or more dose administrations daily, for one or several days.

In some examples, the mRNA is administered at an initial (or loading) dose which is higher than subsequent (maintenance doses). For example, the mRNA is administered at an initial dose of between about 10 mg/kg to about 30 mg/kg. The mRNA is then administered at a maintenance dose of between about 0.0001 mg/kg to about 10 mg/kg. The maintenance doses may be administered every 7-35 days, such as, every 7 or 14 or 28 days.

In some examples, a dose escalation regime is used, in which the mRNA is initially administered at a lower dose than used in subsequent doses. This dosage regime is useful in the case of subject's initially suffering adverse events

In the case of a subject that is not adequately responding to treatment, multiple doses in a week may be administered. Alternatively, or in addition, increasing doses may be administered.

A subject may be retreated with the enriched LNP population of the present disclosure. A subject may be retreated with the enriched LNP population, by being given more than one exposure or set of doses, such as at least about two exposures of the LNP population, for example, from about 2 to 60 exposures, and more particularly about 2 to exposures, most particularly, about 2 to 20 exposures.

In one example, any retreatment may be given when signs or symptoms of disease return. In another example, any retreatment may be given at defined intervals. For example, subsequent exposures may be administered at various intervals, such as, for example, about 24-28 weeks or 48-56 weeks or longer. For example, such exposures are administered at intervals each of about 12-14 weeks, 24-26 weeks or about 38-42 weeks, or about 50-54 weeks.

In the case of a subject that is not adequately responding to treatment, multiple doses in a week may be administered. Alternatively, or in addition, increasing doses may be administered.

In another example, for subjects experiencing an adverse reaction, the initial (or loading) dose may be split over numerous days in one week or over numerous consecutive days.

In one example, the pharmaceutical composition or vaccine as described herein described herein may be administered as part of a regimen. In one example, a regimen administered to a subject may comprise or consist of a single dose. In one example, a regimen administered to a subject may comprise a plurality of doses (e.g., at least two doses, at least three doses, or more). In one example, a regimen administered to a subject may comprise a first dose and a second dose. In one example, the regimen consists of administration of two doses of the composition. In one example, the first and second dose are given at least 2 weeks apart, at least 3 weeks apart, at least 4 weeks apart, or more. In one example, such doses may be at least 1 month, at least 2 months, at least 3 months, at least 4 months, at least 5 months, at least 6 months, at least 7 months, at least 8 months, at least 9 months, at least 10 months, at least 11 months, at least 12 months, or more apart. In one example, doses may be administered days apart, such as 1, 2, 3, 4, 5, 6, 7, 8, 9,10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 or more days apart. In one example, doses may be administered about 1 to about 3 weeks apart, or about 1 to about 4 weeks apart, or about 1 to about 5 weeks apart, or about 1 to about 6 weeks apart, or about 1 to more than 6 weeks apart. In one example, doses may be separated by a period of about 7 to about 60 days, such as for example about 14 to about 48 days, etc. In one example, a minimum number of days between doses may be about 1, 2, 3, 4, 5, 6, 7, 8, 9, 10, 11, 12, 13, 14, 15, 16, 17, 18, 19, 20, 21 or more. In one example, a maximum number of days between doses may be about 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, or fewer. In one example, doses may be about 21 to about 28 days apart. In one example, doses may be about 21 to about 56 days apart. In one example, the first dose is a different amount than the one or more subsequent doses. In one example, a composition described herein is administered (e.g., by intramuscular injection) as a series of two doses 21 days apart. In one example, a composition described herein is administered (e.g., by intramuscular injection) as a series of two doses 56 days apart.

Each dose may contain an amount of RNA that provides a therapeutically effective amount. In one example, a dose may contain sufficient RNA to induce an immune response in a subject administered at least one dose of the composition. In one example, a dose may comprise from 0.0001 μg to 300 μg, 0.001 μg to 200 μg, or 0.001 μg to 100 μg, such as about 0.001 μg, about 0.01 μg, about 0.1 μg, about 1 μg, about 3 μg, about 10 μg, about 30 μg, about 50 μg, or about 100 μg of RNA. In one example, a dose may comprise 100 μg or lower, 90 μg or lower, 80 μg or lower, 70 μg or lower, 60 μg or lower, 50 μg or lower, 40 μg or lower, 30 μg or lower, 20 μg or lower, 10 μg or lower, 5 μg or lower, 2.5 μg or lower, or 1 μg or lower of RNA. In one example, a dose may comprise at least 0.001 μg, at least 0.01 μg, at least 0.1 μg, at least 0.25 μg, at least 0.5 μg, at least 1 μg, at least 2 μg, at least 3 μg, at least 4 μg, at least 5 μg, at least 10 μg, at least 20 μg, at least 30 μg, or at least 40 μg of RNA. In one example, an effective amount is about 100 μg RNA per dose. In one example, an effective amount is about 30 μg RNA per dose. In one example, an effective amount is about 10 μg RNA per dose. In one example, an effective amount is about 5 μg RNA per dose. In one example, an effective amount is about 3 μg RNA per dose. In one example, an effective amount is about 1 μg RNA per dose. In one example, at least two of such doses are administered.

In one example, the mRNA is administered to a subject at a dose of 100 μg or less, 90 μg or less, 80 μg or less, 70 μg or less, 60 μg or less, 50 μg or less, 40 μg or less, 30 μg or less, 20 μg or less, 10 μg or less or 5 μg or less. In one example, the mRNA is administered to a subject at a dose of 10 μg or less.

Without wishing to be bound by any particular theory, the present disclosure suggests that an enriched LNP population produced using the methods described herein may be particularly useful and/or effective for use as or in an immunogenic composition (e.g., a vaccine), and/or for achieving immunological effects as described herein (e.g., generation of neutralizing antibodies, and/or T cell responses (e.g., CD4+ and/or CD8+ T cell responses)).

In one example, the amount of mRNA administered (i.e. dose) is effective to induce in the subject an immune response, wherein the amount of RNA administered is sufficient to induce an immune response in the subject at an at least 2-fold (including, e.g., at least 3-fold, at least 4-fold, at least 5-fold, at least 10 fold) lower dose relative to a reference composition that has not been treated with an anion exchanger. In one example, the subject is a mouse model.

In one example, the dose comprises less than 100 μg (e.g. less the 50 μg, less than 40 μg or less than 30 ug) of mRNA and the composition elicits an immune response that is greater than the immune response elicited by a reference composition comprising at least 100 μg of mRNA that has not been treated with an anion exchanger

In one example, the immune response may comprise generation of a binding antibody titer against the one or more antigens encoded by the mRNA (for example a coronavirus protein or a fragment thereof or an influenza protein or fragment thereof). In one example, an immune response may comprise generation of a binding antibody titer against the spike (S) protein and/or a nucleocapsid (N) protein of a coronavirus (e.g. a SARS-CoV-2 N protein and/or a S protein). In one example, an immune response may comprise generation of a binding antibody titer against the SARS-CoV-2 N protein and/or a S protein from SARS-CoV-2 strain 2019-nCoV/USA-WA1/2020. In one example, an immune response may comprise generation of a binding antibody titer against an influenza A virus strain protein (for example, an influenza A virus hemagglutinin (HA) protein, a neuraminidase (NA) protein, a matrix (M) protein, a nucleoprotein (NP), a non-structural (NS) protein, or an immunogenic fragment or variant thereof). In one example, an immune response may comprise generation of a binding antibody titer against a H5 hemagglutinin protein, M1 matrix protein and/or a N1 neuraminidase protein.

In one example, an immune response may comprise generation of a neutralizing antibody titer against the one or more antigens encoded by the mRNA (for example a coronavirus protein or a fragment thereof or an influenza protein or fragment thereof). In one example, an immune response may comprise generation of a neutralizing antibody titer against the spike (S) protein and/or a nucleocapsid (N) protein of a coronavirus (e.g. a SARS-CoV-2 N protein and/or a S protein). In one example, an immune response may comprise generation of a neutralizing antibody titer against the SARS-CoV-2 N protein and/or a S protein from SARS-CoV-2 strain 2019-nCoV/USA-WA1/2020. In one example, an immune response may comprise generation of a neutralizing antibody titer against an influenza A virus strain protein (for example, an influenza A virus hemagglutinin (HA) protein, a neuraminidase (NA) protein, a matrix (M) protein, a nucleoprotein (NP), a non-structural (NS) protein, or an immunogenic fragment or variant thereof). In one example, an immune response may comprise generation of a neutralizing antibody titer against a H5 hemagglutinin protein, M1 matrix protein and/or a N1 neuraminidase protein. In one example, a composition described herein has been established to achieve a neutralizing antibody titer in an appropriate system (e.g., in a human infected with SARS-CoV-2/influenza and/or a population thereof, and/or in a model system therefor). For example, in some examples, such neutralizing antibody titer may have been demonstrated in one or more of a population of humans, a non-human primate model (e.g., rhesus macaques), and/or a mouse model. In one example, such neutralizing antibody titer may have been demonstrated in a mouse model.

In one example, a neutralizing antibody titer is a titer that is (e.g., that has been established to be) sufficient to reduce viral infection of B cells relative to that observed for an appropriate control (e.g., an unvaccinated control subject, or a subject vaccinated with a live attenuated viral vaccine, an inactivated viral vaccine, or a protein subunit viral vaccine, or a combination thereof). In one such example, such reduction is of at least 30%, 35%, 40%, 45%, 50%, 55%, 60%, 65%, 70%, 75%, 80%, 85%, 90%, 95%, or more.

In one example, a neutralizing antibody titer is a titer that is (e.g., that has been established to be) sufficient to reduce the rate of asymptomatic viral infection relative to that observed for an appropriate control (e.g., an unvaccinated control subject, or a subject vaccinated with a live attenuated viral vaccine, an inactivated viral vaccine, or a protein subunit viral vaccine, or a combination thereof). In one such example, such reduction is of at least 30%, 35%, 40%, 45%, 50%, 55%, 60%, 65%, 70%, 75%, 80%, 85%, 90%, 95%, or more. In one example, such reduction can be characterized by assessment of protein serology, for example, SARS-CoV-2 N protein serology.

In one example, a neutralizing antibody titer is a titer that is (e.g., that has been established to be) sufficient to reduce or block fusion of virus with epithelial cells and/or B cells of a vaccinated subject relative to that observed for an appropriate control (e.g., an unvaccinated control subject, or a subject vaccinated with a live attenuated viral vaccine, an inactivated viral vaccine, or a protein subunit viral vaccine, or a combination thereof). In one such example, such reduction is of at least 30%, 35%, 40%, 45%, 50%, 55%, 60%, 65%, 70%, 75%, 80%, 85%, 90%, 95%, or more.

In one example, induction of a neutralizing antibody titer may be characterized by an elevation in the number of B cells, which in some examples may include plasma cells, class-switched IgG1- and IgG2-positive B cells, and/or germinal center B cells. In some examples, a provided immunogenic composition has been established to achieve such an elevation in the number of B cells in an appropriate system (e.g., in a human infected with SARS-CoV-2/influenza and/or a population thereof, and/or in a model system therefor). For example, such an elevation in the number of B cells may have been demonstrated in one or more of a population of humans, a non-human primate model (e.g., rhesus macaques), and/or a mouse model. In one example, such an elevation in the number of B cells may have been demonstrated in draining lymph nodes and/or spleen of a mouse model after (e.g., at least 7 days, at least 8 days, at least 9 days, at least 10 days, at least 11 days, at least 12 days, at least 13 days, at least 14 days, after) immunization of such a mouse model with a provided immunogenic composition.

In one example, induction of a neutralizing antibody titer may be characterized by a reduction in the number of circulating B cells in blood. In one example, a provided immunogenic composition has been established to achieve such a reduction in the number of circulating B cells in blood of an appropriate system (e.g., in a human infected with SARS-CoV-2/influenza and/or a population thereof, and/or in a model system therefor). For example, such a reduction in the number of circulating B cells in blood may have been demonstrated in one or more of a population of humans, a non-human primate model (e.g., rhesus macaques), and/or a mouse model. In one example, such a reduction in the number of circulating B cells in blood may have been demonstrated in a mouse model after (e.g., at least 7 days, at least 14 days, at least 21 days, at least 28 days, at least 35 days, at least 42 days, at least 49 days, after) immunization of such a mouse model with a composition described herein.

In one example, a regimen as described herein (e.g., one or more doses of a composition described herein) can induce an antibody response in 21 days or less of vaccination. In one example, such an antibody response may comprise a total IgG level as assessed by ELISA of between 100 and 20,000 (e.g. 300 and 10,000) measured at 21 days after vaccination at a dose of 0.001 to 1 μg in an animal model (e.g. mouse model). In one example, a regimen as described herein (e.g., one or more doses of a composition described herein) may induce a pseudovirus-neutralization titer, as measured in an animal model (e.g. mouse model), of between 15,000 and 55,000 42 days after vaccination at a dose of 0.001 to 1 ug. In one example, a regimen as described herein (e.g., one or more doses of a composition described herein) may induce a Hemagglutination inhibition titer (HAI), as measured in an animal model (e.g. mouse model, e.g. BALB/c mice), of greater than 1:40, or greater than 1:80. In one example, a regimen as described herein (e.g., one or more doses of a composition described herein) may induce a Hemagglutination inhibition titer (HAI), as measured in an animal model (e.g. mouse model), of greater than 500, greater than 600, greater than 700, greater than 800, greater than 900, 42 days after vaccination.

In one example, a regimen as described herein (e.g., one or more doses of a composition described herein) may expand antigen-specific CD8 and/or CD4 T cell response by at least at 50% or more (including, e.g., at least 60%, at least 70%, at least 80%, at least 90%, at least 95%, or more), as compared to that observed in absence of such a composition. In one example, a regimen as described herein (e.g., one or more doses of a composition described herein) may expand antigen-specific CD8 and/or CD4 T cell response by at least at 1.5-fold or more (including, e.g., at least 2-fold, at least 3-fold, at least 5-fold, at least 10-fold, at least 50-fold, at least 100-fold, at least 500-fold, at least 1000-fold, or more), as compared to that observed in absence of such a composition.

In one example, a regimen as described herein (e.g., one or more doses of a composition described herein) may expand T cells that exhibit a T h phenotype (e.g., as characterized by expression of IFN-gamma, IL-2, IL-4, and/or IL-5) by at least at 50% or more (including, e.g., at least 60%, at least 70%, at least 80%, at least 90%, at least 95%, or more), as compared to that observed in absence of such a composition. In one example, a regimen as described herein (e.g., one or more doses of a composition described herein) may expand T cells that exhibit a Th1 phenotype (e.g., as characterized by expression of IFN-gamma, IL-2, IL-4, and/or IL-5), for example by at least at 1.5-fold or more (including, e.g., at least 2-fold, at least 3-fold, at least 5-fold, at least 10-fold, at least 50-fold, at least 100-fold, at least 500-fold, at least 1000-fold, or more), as compared to that observed in absence of such a composition. In some examples, a T-cell phenotype may be or comprise a Th1-dominant cytokine profile (e.g., as characterized by INF-gamma positive and/or IL-2 positive), and/or no by or biologically insignificant IL-4 secretion.

In one example, a regimen as described herein (e.g., one or more doses of a composition described herein) induces and/or achieves production of antigen specific CD4+ T cells. In one example, characterization of CD4+ and/or CD8+ T cell responses (e.g., described herein) in subjects receiving a composition described herein may be performed using ex vivo assays using PBMCs collected from the subjects. In one example, immunogenicity of mRNA compositions described herein may be assessed by one of or more of the following serological immunogenicity assays: detection of IgG, IgM, and/or IgA to the mRNA encoded protein(s) present in blood samples of a subject receiving a provided mRNA composition, and/or neutralization assays using an appropriate pseudovirus and/or a wild-type virus.

In one example, the compositions described herein (e.g., when administered to a relevant population) may provide improved therapeutic outcomes (e.g., effective immune responses as described herein and/or detectable expression of encoded protein or an immunogenic fragment thereof) with one or more doses relative to a composition that is not treated with an anion exchanger prior to administration. In an example, a particular outcome may be achieved at a lower dose (e.g. a 0.001 μg dose in a mouse model) than required for a composition which is not treated with an anion exchanger prior to administration.

In one example, the compositions and/or methods described herein may provide an antigen neutralizing geometric mean titer, as measured at 42 days after a first dose or 21 days after a second dose, that is at least 1.5-fold or higher (including, e.g., at least 2-fold, at least 2.5-fold, at least 3-fold, at least 3.5-fold or higher), as compared to a neutralizing GMT of a control composition which has not been treated with an anion exchanger. In an example, the increase in antigen neutralizing geometric mean titer may be achieved at a low dose (e.g. a 0.001 μg dose in a mouse model).

In one example, the compositions and/or methods described herein may provide an in vitro potency that is at least 1.5-fold or higher (including, e.g., at least 2-fold, at least 3-fold, at least 4-fold, at least 4.5-fold, at least 5-fold, at least 8-fold, at least 10-fold or higher), as compared to an in vitro potency of the equivalent composition which has not been treated with an anion exchanger. In an example, the increase in in vitro potency may be achieved at a low dose (e.g. a 0.001 μg dose in a mouse model). In vitro potency may be measured using any method known to the person skilled in the art. In one example, the in vitro potency is measured as described herein.

In one example, the binding and/or neutralizing antibody titer produced in a mouse vaccinated with at least one dose of the compositions described herein is increased by at least 1 log relative to a control, wherein the control is the binding and/or neutralizing antibody titer produced in a mouse who has been administered the composition that has not be contacted with an anion exchanger. In an example, the increase in binding and/or neutralizing antibody titer may be achieved at a low dose (e.g. a 0.001 μg dose).

In one example, the binding and/or neutralizing antibody titer produced in a mouse vaccinated with at least one dose of the compositions described herein is increased at least 2 times relative to a control, wherein the control is the binding and/or neutralizing antibody titer produced in a mouse who has been administered the composition that has not be contacted with an anion exchanger. In an example, the increase in binding and/or neutralizing antibody titer may be achieved at a low dose (e.g. a 0.001 μg dose).

### Methods of Treatment or Prevention

Diseases, disorders, and/or conditions may be treated and/or prevented by a composition comprising an enriched population of LNP described herein. Such diseases, disorders, and/or conditions may include, but are not limited to, rare diseases, infectious diseases, cancer and proliferative diseases, genetic diseases, autoimmune diseases, diabetes, neurodegenerative diseases, cardio- and reno-vascular diseases, and metabolic diseases.

LNP compositions may be formulated in unit dosage form. The therapeutically effective or prophylactically effective dose for any particular patient will depend upon a variety of factors including the severity and identify of a disorder being treated; the specific composition employed; the age, body weight, general health, sex, and diet of the patient; the time of administration, route of administration, and rate of excretion of the specific pharmaceutical composition employed; the duration of the treatment; drugs used in combination or coincidental with the specific pharmaceutical composition employed; and like factors well known in the medical arts.

LNP compositions described herein may be used in combination with one or more other therapeutic, prophylactic, diagnostic, or imaging agents. They may be administered together in a single composition or administered separately in different compositions.

The enriched population of LNP described herein may be used in methods of producing a polypeptide of interest in a mammalian cell. Methods of producing polypeptides involve contacting a cell with the enriched population of LNP, as described herein, including an mRNA encoding the polypeptide of interest. Upon contacting the cell with the enriched population of LNP, the mRNA may be taken up and translated in the cell to produce the polypeptide of interest.

The step of contacting the enriched population of LNP with a cell may involve or cause transfection. A phospholipid included in the lipid component of the LNP composition may facilitate transfection and/or increase transfection efficiency, for example, by interacting and/or fusing with a cellular or intracellular membrane. Transfection may allow for the translation of the mRNA within the cell.

In some examples, the LNP compositions described herein may be used therapeutically. For example, an mRNA included in the LNP composition may encode a therapeutic polypeptide (e.g., in a translatable region) and produce the therapeutic polypeptide upon contacting and/or entry (e.g., transfection) into a cell. In other examples, an mRNA included in the LNP composition may encode a polypeptide that may improve or increase the immunity of a subject.

In examples, an mRNA included in an LNP composition may encode a recombinant polypeptide that may replace one or more polypeptides that may be substantially absent in a cell contacted with the LNP composition. The one or more substantially absent polypeptides may be lacking due to a genetic mutation of the encoding gene or a regulatory pathway thereof. Alternatively, a recombinant polypeptide produced by translation of the mRNA may antagonize the activity of an endogenous protein present in, on the surface of, or secreted from the cell. An antagonistic recombinant polypeptide may be desirable to combat deleterious effects caused by activities of the endogenous protein, such as altered activities or localization caused by mutation. In another alternative, a recombinant polypeptide produced by translation of the mRNA may indirectly or directly antagonize the activity of a biological moiety present in, on the surface of, or secreted from the cell. Antagonized biological moieties may include, but are not limited to, lipids (e.g., cholesterol), lipoproteins (e.g., low density lipoprotein), nucleic acids, carbohydrates, and small molecule toxins. Recombinant polypeptides produced by translation of the mRNA may be engineered for localization within the cell, such as within a specific compartment such as the nucleus, or may be engineered for secretion from the cell or for translocation to the plasma membrane of the cell.

In some examples, contacting a cell with an LNP composition including an mRNA may reduce the innate immune response of a cell to an exogenous polynucleotide. A cell may be contacted with a first LNP composition including a first amount of a first exogenous mRNA including a translatable region and the level of the innate immune response of the cell to the first exogenous mRNA may be determined. Subsequently, the cell may be contacted with a second LNP composition including a second amount of the first exogenous mRNA, the second amount being a lesser amount of the first exogenous mRNA compared to the first amount. Alternatively, the second composition may include a first amount of a second exogenous mRNA that is different from the first exogenous mRNA. The steps of contacting the cell with the first and second LNP compositions may be repeated one or more times. Additionally, efficiency of polypeptide production (e.g., translation) in the cell may be optionally determined, and the cell may be re-contacted with the first and/or second composition repeatedly until a target protein production efficiency is achieved.

In some examples, the present disclosure provides for the use of a composition comprising an enriched population of LNP produced by the methods described herein in the manufacture of a medicament for the treatment of a disease, disorder or condition. The disease, disorder or condition may be as described in any one or more examples herein.

The medicament may be for the prevention or treatment of a cancer, an infectious disease, an allergy, or an autoimmune disease. In examples, the medicament is a vaccine. The vaccine may be a tumor vaccine, an influenza vaccine, or a SARS-CoV-2 vaccine.

The present disclosure also provides methods of treating or preventing or delaying progression of a disease or condition in a subject comprising administering the enriched population of LNP or a composition comprising the enriched population of LNP. For example, the disease or condition is selected from the group consisting of SARS-CoV-2 infection, COVID-19, ARDS and combinations thereof.

In one example there is provided a method of generating an immune response in a subject, the method comprising administering to the subject an enriched population of LNPs in an amount of less than 10 μg RNA, wherein the LNPs comprise an ionizable lipid, a phospholipid, a PEG lipid, and a structural lipid, wherein at least 50% of the LNPs comprise RNA encapsulated within the LNP. In one example, at least 60%, at least 70% at least 80%, at least 90%, at least 95%, at least 98% or at least 99% of the LNPs comprise RNA encapsulated within the LNP.

### Use of LNPs in a Vaccine

The enriched population of LNP produced using the methods described herein may be a component of a vaccine. In one example, the present disclosure provides methods of using the pharmaceutical composition of the present disclosure as a vaccine. Vaccines include compounds and preparations that are capable of providing immunity against one or more conditions related to infectious diseases and so may include mRNAs encoding infectious disease derived antigens and/or epitopes. Vaccines also include compounds and preparations that direct an immune response against cancer cells and can include mRNAs encoding tumor cell derived antigens, epitopes, and/or neoepitopes. Compounds eliciting immune responses may include vaccines, corticosteroids (e.g., dexamethasone), and other species.

In examples, the mRNA encodes an antigenic peptide or protein, or a fragment, variant or derivative thereof. The antigenic peptides or proteins may be pathogenic antigens, tumour antigens, allergenic antigens or autoimmune self-antigens. Such pathogenic antigens may be those derived from pathogenic organisms, in particular bacterial, viral or protozoological (multicellular) pathogenic organisms, which evoke an immunological reaction in a mammalian subject, such as a human. Pathogenic antigens may be surface antigens, for example proteins or fragments thereof, located at the surface of the virus or the bacterial or protozoological organism.

Pathogenic antigens of interest may include those derived from one or more of: Acinetobacter baumannii, Anaplasma genus, Anaplasma phagocytophilum, Ancylostoma braziliense, Ancylostoma duodenale, Area nobacteri urn haemolyticum, Ascaris lumbricoides, Aspergillus genus, Astroviridae, Babesia genus, Bacillus anthracis, Bacillus cereus, Bartonella henselae, BK virus, Blastocysts hominis, Blastomyces dermatitidis, Bordetella pertussis, Borrelia burgdorferi, Borrelia genus, Borrelia spp, Brucella genus, Brugia malayi, Bunyaviridae family, Burkholderia cepacia and other Burkholderia species, Burkholderia mallei, Burkholderia pseudomallei, Caliciviridae family, Campylobacter genus, Candida albicans, Candida spp, Chlamydia trachomatis, Chlamydophila pneumoniae, Chlamydophila psittaci, QD prion, Clonorchis sinensis, Clostridium botulinum, Clostridium difficile, Clostridium perfringens, Clostridium perfringens, Clostridium spp, Clostridium tetani, Coccidioides spp, coronavi ruses, Corynebacterium diphtheriae, Coxiella burnetii, Crimean-Congo hemorrhagic fever virus, Cryptococcus neoformans, Cryptosporidium genus, Cytomegalovirus (CMV), Dengue viruses (DEN-1, DEN-2, DEN-3 and DEN-4), Dientamoeba fragilis, Ebolavirus (EBOV), Echinococcus genus, Ehrlichia chaffeensis, Ehrlichia ewingii, Ehrlichia genus, Entamoeba histolytica, Enterococcus genus, Enterovirus genus, Enteroviruses, mainly Coxsackie A virus and Enterovirus 71 (EV71), Epidermophyton spp, Epstein-Barr Virus (EBV), Escherichia coli O157:H7, Oll1 and O104: H4, Fasciola hepatica and Fasciola gigantica, FFI prion, Filarioidea superfamily, Flaviviruses, Francisella tularensis, Fusobacterium genus, Geotrichum candidum, Giardia intestinalis, Gnathostoma spp, GSS prion, Guanarito virus, Haemophilus ducreyi, Haemophilus influenzae, Helicobacter pylori, Henipavirus (Hendra virus Nipah virus), Hepatitis A Virus, Hepatitis B Virus (HBV), Hepatitis C Virus (HCV), Hepatitis D Virus, Hepatitis E Virus, Herpes simplex virus 1 and 2 (HSV-1 and HSV-2), Histoplasma capsulatum, HIV (Human immunodeficiency virus), Hortaea werneckii, Human bocavirus (HBoV), Human herpesvirus 6 (HHV-6) and Human herpesvirus 7 (HHV-7), Human metapneumovirus (hMPV), Human papillomavirus (HPV), Human parainfluenza viruses (HPIV), Japanese encephalitis virus, JC virus, Junin virus, Kingella kingae, Klebsiella granulomatis, Kuru prion, Lassa virus, Legionella pneumophila, Leishmania genus, Leptospira genus, Listeria monocytogenes, Lymphocytic choriomeningitis virus (LCMV), Machupo virus, Malassezia spp, Marburg virus, Measles virus, Metagonimus yokagawai, Microsporidia phylum, Molluscum contagiosum virus (MCV), Mumps virus, Mycobacterium leprae and Mycobacterium lepromatosis, Mycobacterium tuberculosis, Mycobacterium ulcerans, Mycoplasma pneumoniae, Naegleria fowleri, Necator americanus, Neisseria gonorrhoeae, Neisseria meningitidis, Nocardia asteroides, Nocardia spp, Onchocerca volvulus, Orientia tsutsugamushi, Orthomyxoviridae family (Influenza), Paracoccidioides brasiliensis, Paragonimus spp, Paragonimus westermani, Parvovirus B19, Pasteurella genus, Plasmodium genus, Pneumocystis jirovecii, Poliovirus, Rabies virus, Respiratory syncytial virus (RSV), Rhinovirus, rhinoviruses, Rickettsia akari, Rickettsia genus, Rickettsia prowazekii, Rickettsia rickettsii, Rickettsia typhi, Rift Valley fever virus, Rotavirus, Rubella virus, Sabia virus, Salmonella genus, Sarcoptes scabiei, SARS coronavirus, Schistosoma genus, Shigella genus, Sin Nombre virus, Hantavirus, Sporothrix schenckii, Staphylococcus genus, Staphylococcus genus, Streptococcus agalactiae, Streptococcus pneumoniae, Streptococcus pyogenes, Strongyloides stercoralis, Taenia genus, Taenia solium, Tick-borne encephalitis virus (TBEV), Toxocara canis or Toxocara cati, Toxoplasma gondii, Treponema pallidum, Trichinella spiralis, Trichomonas vaginalis, Trichophyton spp, Trichuris trichiura, Trypanosoma brucei, Trypanosoma cruzi, Ureaplasma urealyticum, Varicella zoster virus (VZV), Varicella zoster virus (VZV), Variola major or Variola minor, vCJD prion, Venezuelan equine encephalitis virus, Vibrio cholerae, West Nile virus, Western equine encephalitis virus, Wuchereria bancrofti, Yellow fever virus, Yersinia enterocolitica, Yersinia pestis, and Yersinia pseudotuberculosis.

In certain examples, relevant antigens may be derived from the pathogens selected from: Severe Acute Respiratory Syndrome Coronavirus and Severe Acute Respiratory Syndrome Coronavirus 2 (SARS-CoV-1 and SARS-CoV-2), Middle East respiratory syndrome coronavirus (MERS-CoV), Influenza virus, respiratory syncytial virus (RSV), Herpes simplex virus (HSV), human Papilloma virus (HPV), Human immunodeficiency virus (HIV), Plasmodium, Staphylococcus aureus, Dengue virus, Chlamydia trachomatis, Cytomegalovirus (CMV), Hepatitis B virus (HBV), Mycobacterium tuberculosis, Rabies virus, and Yellow Fever Virus.

In some examples, the relevant pathogenic antigen may be selected from: Outer membrane protein A OmpA, biofilm associated protein Bap, transport protein MucK (Acinetobacter baumannii, Acinetobacter infections)); variable surface glycoprotein VSG, microtubule-associated protein MAPP15, trans-sialidase TSA (Trypanosoma brucei, African sleeping sickness (African trypanosomiasis)); HIV p24 antigen, HIV envelope proteins (Gp120, Gp41, Gp160), polyprotein GAG, negative factor protein Nef, transactivator of transcription Tat (HIV (Human immunodeficiency virus), AIDS (Acquired immunodeficiency syndrome)); galactose-inhibitable adherence protein GIAP, 29 kDa antigen Eh29, Gal/GalNAc lectin, protein CRT, 125 kDa immunodominant antigen, protein M17, adhesin ADH112, protein STIRP (Entamoeba histolytica, Amoebiasis); Major surface proteins 1-5 (MSPla, MSPlb, MSP2, MSP3, MSP4, MSP5), type IV secreotion system proteins (VirB2, VirB7, VirBll, VirD4) (Anaplasma genus, Anaplasmosis); protective Antigen PA, edema factor EF, lethal facotor LF, the S-layer homology proteins SLH (Bacillus anthracis, Anthrax); acranolysin, phospholipase D, collagen-binding protein CbpA (Area nobacteri um haemolyticum, Area nobacteri um haemolyticum infection); nucleocapsid protein NP, glycoprotein precursor GPC, glycoprotein GP1, glycoprotein GP2 (Junin virus, Argentine hemorrhagic fever); chitin-protein layer proteins, 14 kDa surface antigen A14, major sperm protein MSP, MSP polymerization-organizing protein MPOP, MSP fiber protein 2 MFP2, MSP polymerization-activating kinase MPAK, ABA-1-like protein ALB, protein ABA-1, cuticulin CUT-1 (Ascaris lumbricoides, Ascariasis); 41 kDa allergen Asp vA3, allergen Asp f3, major conidial surface protein rodlet A, protease Peplp, GPI-anchored protein Gellp, GPI-anchored protein Crflp (Aspergillus genus, Aspergillosis); family VP26 protein, VP29 protein (Astroviridae, Astrovirus infection); Rhoptry-associated protein 1 RAP-1, merozoite surface antigens MSA-1, MSA-2 (a1, a2, b, c), 12D3, 11C5, 21B4, P29, variant erythrocyte surface antigen VESA1, Apical Membrane Antigen 1 AMA-1 (Babesia genus, Babesiosis); hemolysin, enterotoxin C, PXO1-51, glycolate oxidase, ABC-transporter, penicillin-binding protein, zinc transporter family protein, pseudouridine synthase Rsu, plasmid replication protein RepX, oligoendopeptidase F, prophage membrane protein, protein HemK, flagellar antigen H, 28.5-kDa cell surface antigen (Bacillus cereus, Bacillus cereus infection); large T antigen LT, small T antigen, capsid protein VP1, capsid protein VP2 (BK virus, BK virus infection); 29 kDa-protein, caspase-3-like antigens, glycoproteins (Blastocysts hominis, Blastocystis hominis infection); yeast surface adhesin WI-1 (Blastomyces dermatitidis, Blastomycosis); nucleoprotein N, polymerase L, matrix protein Z, glycoprotein GP (Machupo virus, Bolivian hemorrhagic fever); outer surface protein A OspA, outer surface protein OspB, outer surface protein OspC, decorin binding protein A DbpA, decorin binding protein B DbpB, flagellar filament 41 kDa core protein Fla, basic membrane protein A precursor BmpA (Immunodominant antigen P39), outer surface 22 kDa lipoprotein precursor (antigen IPLA7), variable surface lipoprotein vlsE (Borrelia genus, Borrelia infection); Botulinum neurotoxins BoNT/A1, BoNT/A2, BoNT/A3, BoNT/B, BoNT/C, BoNT/D, BoNT/E, BoNT/F, BoNT/G, recombinant botulinum toxin F He domain FHc (Clostridium botulinum, Botulism (and Infant botulism)); nucleocapsid, glycoprotein precursor (Sabia virus, Brazilian hemorrhagic fever); copper/Zinc superoxide dismutase SodC, bacterioferritin Bfr, 50S ribosomal protein RpIL, OmpA-like transmembrane domain-containing protein Omp31, immunogenic 39-kDa protein M5 P39, zinc ABC transporter periplasmic zinc-binding protein znuA, periplasmic immunogenic protein Bp26, 30S ribosomal protein S12 RpsL, glyceraldehyde-3-phosphate dehydrogenase Gap, kDa outer-membrane immunogenic protein precursor Omp25, invasion protein B lalB, trigger factor Tig, molecular chaperone DnaK, putative peptidyl-prolyl cis-trans isomerase SurA, lipoprotein Omp19, outer membrane protein MotY Omp16, conserved outer membrane protein D15, malate dehydrogenase Mdh, component of the Type-IV secretion system (T4SS) VirJ, lipoprotein of unknown function BAB1_0187 (Brucella genus, Brucellosis); members of the ABC transporter family (LolC, OppA, and PotF), putative lipoprotein releasing system transmembrane protein LolC/E, flagellin FliC, Burkholderia intracellular motility A BimA, bacterial Elongation factor-Tu EF-Tu, 17 kDa OmpA-like protein, boaA coding protein, boaB coding protein (Burkholderia cepacia and other Burkholderia species, Burkholderia infection); mycolyl-transferase Ag85A, heat-shock protein Hsp65, protein TB10.4, 19 kDa antigen, protein PstS3, heat-shock protein Hsp70 (Mycobacterium ulcerans, Buruli ulcer); norovirus major and minor viral capsid proteins VP1 and VP2, genome polyprotein, Sapoviurus capsid protein VP1, protein Vp3, genome polyprotein (Caliciviridae family, Calicivirus infection (Norovirus and Sapovirus)); major outer membrane protein PorA, flagellin FlaA, surface antigen CjaA, fibronectin binding protein CadF, aspartate/glutamate-binding ABC transporter protein PeblA, protein FspA1, protein FspA2 (Campylobacter genus, Campylobacteriosis); glycolytic enzyme enolase, secreted aspartyl proteinases SAPI-10, glycophosphatidylinositol (GPI)-linked cell wall protein, protein Hyrl, complement receptor 3-related protein CR3-RP, adhesin Als3p, heat shock protein 90 kDa hsp90, cell surface hydrophobicity protein CSH (usually Candida albicans and other Candida species, Candidiasis); 17-kDa antigen, protein P26, trimeric autotransporter adhesins TAAs, Bartonella adhesin A BadA, variably expressed outer-membrane proteins Vomps, protein Pap3, protein HbpA, envelope-associated protease HtrA, protein OMP89, protein GroEL, protein LalB, protein OMP43, dihydrolipoamide succinyltransferase SucB (Bartonella henselae, Cat-scratch disease); amastigote surface protein-2, amastigote-specific surface protein SSP4, cruzipain, trans-sialidase TS, trypomastigote surface glycoprotein TSA-1, complement regulatory protein CRP-10, protein G4, protein G2, paraxonemal rod protein PAR2, paraflagellar rod component Pari, mucin-Associated Surface Proteins MPSP (Trypanosoma cruzi, Chagas Disease (American trypanosomiasis)); envelope glycoproteins (gB, gC, gE, gH, gl, gK, gL), (Varicella zoster virus (VZV), Chickenpox); major outer membrane protein MOMP, probable outer membrane protein PMPC, outer membrane complex protein B OmcB, heat shock proteins Hsp60 HSP10, protein IncA, proteins from the type III secretion system, ribonucleotide reductase small chain protein NrdB, plasmid protein Pgp3, chlamydial outer protein N CopN, antigen CT521, antigen CT425, antigen CT043, antigen TC0052, antigen TC0189, antigen TC0582, antigen TC0660, antigen TC0726, antigen TC0816, antigen TC0828 (Chlamydia trachomatis, Chlamydia); low calcium response protein E LCrE, chlamydial outer protein N CopN, serine/threonine-protein kinase PknD, acyl-carrier-protein S-malonyltransferase FabD, single-stranded DNA-binding protein Ssb, major outer membrane protein MOMP, outer membrane protein 2 Omp2, polymorphic membrane protein family (Pmp1, Pmp2, Pmp3, Pmp4, Pmp5, Pmp6, Pmp7, Pmp8, Pmp9, Pmp10, Pmp11, Pmp12, Pmp13, Pmp14, Pmp15, Pmp16, Pmp17, Pmp18, Pmp19, Pmp20, Pmp21), (Chlamydophila pneumoniae, Chlamydophila pneumoniae infection); cholera toxin B CTB, toxin coregulated pilin A TcpA, toxin coregulated pilin TcpF, toxin co-regulated pilus biosynthesis protein F TcpF, cholera enterotoxin subunit A, cholera enterotoxin subunit B, Heat-stable enterotoxin ST, mannose-sensitive hemagglutinin MSHA, outer membrane protein U Porin ompU, Poring B protein, polymorphic membrane protein-D (Vibrio cholerae, Cholera); propionyl-CoA carboxylase PCC, 14-3-3 protein, prohibitin, cysteine proteases, glutathione transferases, gelsolin, cathepsin L proteinase CatL, Tegumental Protein 20.8 kDa TP20.8, tegumental protein 31.8 kDa TP31.8, lysophosphatidic acid phosphatase LPAP, (Clonorchis sinensis, Clonorchiasis); surface layer proteins SLPs, glutamate dehydrogenase antigen GDH, toxin A, toxin B, cysteine protease Cwp84, cysteine protease Cwpl3, cysteine protease Cwpl9, Cell Wall Protein CwpV, flagellar protein FliC, flagellar protein FliD (Clostridium difficile, Clostridium difficile infection); rhinoviruses: capsid proteins VP1, VP2, VP3, VP4; coronaviruses: sprike proteins S, envelope proteins E, membrane proteins M, nucleocapsid proteins N (usually rhinoviruses and coronaviruses, Common cold (Acute viral rhinopharyngitis; Acute coryza)); prion protein Prp (CJD prion, Creutzfeldt-Jakob disease (CJD)); envelope protein Gc, envelope protein Gn, nucleocapsid proteins (Crimean-Congo hemorrhagic fever virus, Crimean-Congo hemorrhagic fever (CCHF)); virulence-associated DEAD-box RNA helicase VAD1, galactoxylomannan-protein GalXM, glucuronoxylomannan GXM, mannoprotein MP (Cryptococcus neoformans, Cryptococcosis); acidic ribosomal protein P2 CpP2, mucin antigens Muc1, Muc2, Muc3 Muc4, Muc5, Muc6, Muc7, surface adherence protein CP20, surface adherence protein CP23, surface protein CP12, surface protein CP21, surface protein CP40, surface protein CP60, surface protein CP15, surface-associated glycopeptides gp40, surface-associated glycopeptides gp15, oocyst wall protein AB, profilin PRF, apyrase (Cryptosporidium genus, Cryptosporidiosis); fatty acid and retinol binding protein-1 FAR-1, tissue inhibitor of metalloproteinase TIMP (TMP), cysteine proteinase ACEY-1, cysteine proteinase ACCP-1, surface antigen Ac-16, secreted protein 2 ASP-2, metalloprotease 1 MTP-1, aspartyl protease inhibitor API-1, surface-associated antigen SAA-1, adult-specific secreted factor Xa serine protease inhibitor anticoagulant AP, cathepsin D-like aspartic protease ARR-1 (usually Ancylostoma braziliense; multiple other parasites, Cutaneous larva migrans (CLM)); cathepsin L-like proteases, 53/25-kDa antigen, 8 kDa family members, cysticercus protein with a marginal trypsin-like activity TsAg5, oncosphere protein TSOL18, oncosphere protein TSOL45-1A, lactate dehydrogenase A LDHA, lactate dehydrogenase B LDHB (Taenia solium, Cysticercosis); pp65 antigen, membrane protein ppl5, capsid-proximal tegument protein ppl50, protein M45, DNA polymerase UL54, helicase UL105, glycoprotein gM, glycoprotein gN, glycoprotein H, glycoprotein B gB, protein UL83, protein UL94, protein UL99 (Cytomegalovirus (CMV), Cytomegalovirus infection); capsid protein C, premembrane protein prM, membrane protein M, envelope protein E (domain I, domain II, domain II), protein NS1, protein NS2A, protein NS2B, protein NS3, protein NS4A, protein 2K, protein NS4B, protein NS5 (Dengue viruses (DEN-1, DEN-2, DEN-3 and DEN-4)-Flaviviruses, Dengue fever); 39 kDa protein (Dientamoeba fragilis, Dientamoebiasis); diphtheria toxin precursor Tox, diptheria toxin DT, pilin-specific sortase SrtA, shaft pilin protein SpaA, tip pilin protein SpaC, minor pilin protein SpaB, surface-associated protein DIP1281 (Corynebacterium diphtheriae, Diphtheria); glycoprotein GP, nucleoprotein NP, minor matrix protein VP24, major matrix protein VP40, transcription activator VP30, polymerase cofactor VP35, RNA polymerase L (Ebolavirus (EBOV), Ebola hemorrhagic fever); prion protein (vQD prion, Variant Creutzfeldt-Jakob disease (vCJD, nvCJD)); UvrABC system protein B, protein Flp1, protein Flp2, protein Flp3, protein TadA, hemoglobin receptor HgbA, outer membrane protein TdhA, protein CpsRA, regulator CpxR, protein SapA, 18 kDa antigen, outer membrane protein NcaA, protein LspA, protein LspA1, protein LspA2, protein LspB, outer membrane component DsrA, lectin DltA, lipoprotein Hip, major outer membrane protein OMP, outer membrane protein OmpA2 (Haemophilus ducreyi, Chancroid); aspartyl protease 1 Pepl, phospholipase B PLB, alpha-mannosidase 1 AMN1, glucanosyltransferase GEL1, urease URE, peroxisomal matrix protein Pmp1, proline-rich antigen Pra, humal T-cell relative protein TcrP (Coccidioides immitis and Coccidioides posadasii, Coccidioidomycosis); allergen Tri r 2, heat shock protein 60 Hsp60, fungal actin Act, antigen Tri r2, antigen Tri r4, antigen Tri t1, protein IV, glycerol-3-phosphate dehydrogenase Gpdl, osmosensor HwSholA, osmosensor HwSholB, histidine kinase HwHhk7B, allergen Mala s 1, allergen Mala s 11, thioredoxin Trx Mala s 13, allergen Mala f, allergen Mala s (usually Trichophyton spp, Epidermophyton spp., Malassezia spp., Hortaea werneckii, Dermatophytosis); protein EG95, protein EG10, protein EG18, protein EgA31, protein EM18, antigen EPC1, antigen B, antigen 5, protein P29, protein 14-3-3, 8-kDa protein, myophilin, heat shock protein 20 HSP20, glycoprotein GP-89, fatty acid binding protein FAPB (Echinococcus genus, Echinococcosis); major surface protein 2 MSP2, major surface protein 4 MSP4, MSP variant SGV1, MSP variant SGV2, outer membrane protein OMP, outer membrande protein 19 OMP-19, major antigenic protein MAPI, major antigenic protein MAP1-2, major antigenic protein MAP1B, major antigenic protein MAP1-3, Erum2510 coding protein, protein GroEL, protein GroES, 30-kDA major outer membrane proteins, GE 100-kDa protein, GE 130-kDa protein, GE 160-kDa protein (Ehrlichia genus, Ehrlichiosis); secreted antigen SagA, sagA-like proteins SalA and SalB, collagen adhesin Scm, surface proteins Fmsl (EbpA(fm), Fms5 (EbpB(fm), Fms9 (EpbC(fm) and FmslO, protein EbpC(fm), 96 kDa immunoprotective glycoprotein G1 (Enterococcus genus, Enterococcus infection); genome polyprotein, polymerase 3D, viral capsid protein VP1, viral capsid protein VP2, viral capsid protein VP3, viral capsid protein VP4, protease 2A, protease 3C (Enterovirus genus, Enterovirus infection); outer membrane proteins OM, 60 kDa outer membrane protein, cell surface antigen OmpA, cell surface antigen OmpB (sca5), 134 kDa outer membrane protein, 31 kDa outer membrane protein, 29.5 kDa outer membrane protein, cell surface protein SCA4, cell surface protein Adr1 (RP827), cell surface protein Adr2 (RP828), cell surface protein SCA1, Invasion protein invA, cell division protein fts, secretion proteins sec O family, virulence proteins virB, tlyA, tlyC, parvulin-like protein Pip, preprotein translocase SecA, 120-kDa surface protein antigen SPA, 138 kD complex antigen, major 100-kD protein (protein I), intracytoplasmic protein D, protective surface protein antigen SPA (Rickettsia prowazekii, Epidemic typhus); Epstein-Barr nuclear antigens (EBNA-1, EBNA-2, EBNA-3A, EBNA-3B, EBNA-3C, EBNA-leader protein (EBNA-LP)), latent membrane proteins (LMP-1, LMP-2A, LMP-2B), early antigen EBV-EA, membrane antigen EBV-MA, viral capsid antigen EBV-VCA, alkaline nuclease EBV-AN, glycoprotein H, glycoprotein gp350, glycoprotein gpllO, glycoprotein gp42, glycoprotein gHgL, glycoprotein gB (Epstein-Barr Virus (EBV), Epstein-Barr Virus Infectious Mononucleosis); capsid protein VP2, capsid protein VP1, major protein NS1 (Parvovirus B19, Erythema infectiosum (Fifth disease)); pp65 antigen, glycoprotein 105, major capsid protein, envelope glycoprotein H, protein U51 (Human herpesvirus 6 (HHV-6) and Human herpesvirus 7 (HHV-7), Exanthem subitum); thioredoxin-glutathione reductase TGR, cathepsins L1 and L2, Kunitz-type protein KTM, leucine aminopeptidase LAP, cysteine proteinase Fas2, saposin-like protein-2 SAP-2, thioredoxin peroxidases TPx, Prx-1, Prx-2, cathepsin I cysteine proteinase CL3, protease cathepsin L CL1, phosphoglycerate kinase PGK, 27-kDa secretory protein, 60 kDa protein HSP35alpha, glutathione transferase GST, 28.5 kDa tegumental antigen 28.5 kDa TA, cathepsin B3 protease CatB3, Type I cystatin stefin-1, cathepsin L5, cathepsin Llg and cathepsin B, fatty acid binding protein FABP, leucine aminopeptidases LAP (Fasciola hepatica and Fasciola gigantica, Fasciolosis); prion protein (FFI prion, Fatal familial insomnia (FFI)); venom allergen homolog-like protein VAL-1, abundant larval transcript ALT-1, abundant larval transcript ALT-2, thioredoxin peroxidase TPX, vespid allergen homologue VAH, thiordoxin peroxidase 2 TPX-2, antigenic protein SXP (peptides N, N1, N2, and N3), activation associated protein-1 ASP-1, Thioredoxin TRX, transglutaminase BmTGA, glutathione-S-transferases GST, myosin, vespid allergen homologue VAH, 175 kDa collagenase, glyceraldehyde-3-phosphate dehydrogenase GAPDH, cuticular collagen Col-4, secreted larval acidic proteins SLAPs, chitinase CHI-1, maltose binding protein MBP, glycolytic enzyme fructose-1,6-bisphosphate aldolase Fba, tropomyosin TMY-1, nematode specific gene product OvB20, onchocystatin CPI-2, Cox-2 (Filarioidea superfamily, Filariasis); phospholipase C PLC, heat-labile enterotoxin B, Iota toxin component 1b, protein CPE1281, pyruvate ferredoxin oxidoreductase, elongation factor G EF-G, perfringolysin 0 Pfo, glyceraldehyde-3-phosphate dehydrogenase GapC, Fructose-bisphosphate aldolase Alf2, Clostridium perfringens enterotoxin CPE, alpha toxin AT, alpha toxoid ATd, epsilon-toxoid ETd, protein HP, large cytotoxin TpeL, endo-beta-N-acetylglucosaminidase Naglu, phosphoglyceromutase Pgm (Clostridium perfringens, Food poisoning by Clostridium perfringens); leukotoxin IktA, adhesion FadA, outer membrane protein RadD, high-molecular weight arginine-binding protein (Fusobacterium genus, Fusobacterium infection); phospholipase C PLC, heat-labile enterotoxin B, Iota toxin component 1b, protein CPE1281, pyruvate ferredoxin oxidoreductase, elongation factor G EF-G, perfringolysin 0 Pfo, glyceraldehyde-3-phosphate dehydrogenase GapC, fructose-bisphosphate aldolase Alf2, Clostridium perfringens enterotoxin CPE, alpha toxin AT, alpha toxoid ATd, epsilon-toxoid ETd, protein HP, large cytotoxin TpeL, endo-beta-N-acetylglucosaminidase Naglu, phosphoglyceromutase Pgm (usually Clostridium perfringens; other Clostridium species, Gas gangrene (Clostridial myonecrosis)); lipase A, lipase B, peroxidase Decl (Geotrichum candidum, Geotrichosis); prion protein (GSS prion, Gerstmann-Straussler-Scheinker syndrome (GSS)); cyst wall proteins CWP1, CWP2, CWP3, variant surface protein VSP, VSP1, VSP2, VSP3, VSP4, VSP5, VSP6, 56 kDa antigen, pyruvate ferredoxin oxidoreductase PFOR, alcohol dehydrogenase E ADHE, alpha-giardin, alpha8-giardin, alphal-guiardin, beta-giardin, cystein proteases, glutathione-S-transferase GST, arginine deiminase ADI, fructose-1,6-bisphosphat aldolase FBA, Giardia trophozoite antigens GTA (GTA1, GTA2), omithine carboxyl transferase OCT, striated fiber-asseblin-like protein SALP, uridine phosphoryl-like protein UPL, alpha-tubulin, beta-tubulin (Giardia intestinalis, Giardiasis); members of the ABC transporter family (LolC, OppA, and PotF), putative lipoprotein releasing system transmembrane protein LolC/E, flagellin FliC, Burkholderia intracellular motility A BimA, bacterial Elongation factor-Tu EF-Tu, 17 kDa OmpA-like protein, boaA coding protein (Burkholderia mallei, Glanders); cyclophilin CyP, 24 kDa third-stage larvae protein GS24, excretion-secretion products ESPs (40, 80, 120 and 208 kDa) (Gnathostoma spinigerum and Gnathostoma hispidum, Gnathostomiasis); pilin proteins, minor pilin-associated subunit pilC, major pilin subunit and variants pilE, pilS, phase variation protein porA, Porin B PorB, protein TraD, Neisserial outer membrane antigen H.8, 70 kDa antigen, major outer membrane protein PI, outer membrane proteins PIA and PIB, W antigen, surface protein A NspA, transferrin binding protein TbpA, transferrin binding protein TbpB, PBP2, mtrR coding protein, ponA coding protein, membrane permease FbpBC, FbpABC protein system, LbpAB proteins, outer membrane protein Opa, outer membrane transporter FetA, iron-repressed regulator MpeR (Neisseria gonorrhoeae, Gonorrhea); outer membrane protein A OmpA, outer membrane protein C OmpC, outer membrane protein K17 OmpK17 (Klebsiella granulomatis, Granuloma inguinale (Donovanosis)); fibronectin-binding protein Sfb, fibronectin/fibrinogen-binding protein FBP54, fibronectin-binding protein FbaA, M protein type 1 Emm1, M protein type 6 Emm6, immunoglobulin-binding protein 35 Sib35, Surface protein R28 Spr28, superoxide dismutase SOD, C5a peptidase ScpA, antigen I/II Agl/II, adhesin AspA, G-related alpha2-macroglobulin-binding protein GRAB, surface fibrillar protein M5 (Streptococcus pyogenes, Group A streptococcal infection); C protein β antigen, arginine deiminase proteins, adhesin BibA, 105 kDA protein BPS, surface antigens c, surface antigens R, surface antigens X, trypsin-resistant protein RI, trypsin-resistant protein R3, trypsin-resistant protein R4, surface immunogenic protein Sip, surface protein Rib, Leucine-rich repeats protein LrrG, serine-rich repeat protein Srr-2, C protein alpha-antigen Bca, Beta antigen Bag, surface antigen Epsilon, alpha-like protein ALP1, alpha-like protein ALP5 surface antigen delta, alpha-like protein ALP2, alphalike protein ALP3, alpha-like protein ALP4, Cbeta protein Bac (Streptococcus agalactiae, Group B streptococcal infection); transferrin-binding protein 2 Tbp2, phosphatase P4, outer membrane protein P6, peptidoglycan-associated lipoprotein Pal, protein D, protein E, adherence and penetration protein Hap, outer membrane protein 26 Omp26, outer membrane protein P5 (Fimbrin), outer membrane protein D15, outer membrane protein OmpP2, 5′-nucleotidase NucA, outer membrane protein PI, outer membrane protein P2, outer membrane lipoprotein Pep, Lipoprotein E, outer membrane protein P4, fuculokinase FucK, [Cu,Zn]-superoxide dismutase SodC, protease HtrA, protein 0145, alpha-galactosylceramide (Haemophilus influenzae, Haemophilus influenzae infection); polymerase 3D, viral capsid protein VP1, viral capsid protein VP2, viral capsid protein VP3, viral capsid protein VP4, protease 2A, protease 3C (Enteroviruses, mainly Coxsackie A virus and Enterovirus 71 (EV71), Hand, foot and mouth disease (HFMD)); RNA polymerase L, protein L, glycoprotein Gn, glycoprotein Gc, nucleocapsid protein S, envelope glycoprotein G1, nucleoprotein NP, protein N, polyprotein M (Sin Nombre virus, Hantavirus, Hantavirus Pulmonary Syndrome (HPS)); heat shock protein HspA, heat shock protein HspB, citrate synthase GltA, protein UreB, heat shock protein Hsp60, neutrophil-activating protein NAP, catalase KatA, vacuolating cytotoxin VacA, urease alpha UreA, urease beta Ureb, protein CpnlO, protein groES, heat shock protein HsplO, protein MopB, cytotoxicity-associated 10 kDa protein CAG, 36 kDa antigen, beta-lactamase HcpA, Beta-lactamase HcpB (Helicobacter pylori, Helicobacter pylori infection); integral membrane proteins, aggregation-prone proteins, 0-antigen, toxin-antigens Stx2B, toxin-antigen Stx1B, adhesion-antigen fragment Int28, protein EspA, protein EspB, Intimin, protein Tir, protein IntC300, protein Eae (Escherichia coli O157:H7, O111 and O104:H4, Hemolytic-uremic syndrome (HUS)); RNA polymerase L, protein L, glycoprotein Gn, glycoprotein Gc, nucleocapsid protein S, envelope glycoprotein G1, nucleoprotein NP, protein N, polyprotein M (Bunyaviridae family, Hemorrhagic fever with renal syndrome (HFRS)); glycoprotein G, matrix protein M, nucleoprotein N, fusion protein F, polymerase L, protein W, proteinC, phosphoprotein p, non-structural protein V (Henipavirus (Hendra virus Nipah virus), Henipavirus infections); polyprotein, glycoprotein Gp2, hepatitis A surface antigen HBAg, protein 2A, virus protein VP1, virus protein VP2, virus protein VP3, virus protein VP4, protein PIB, protein P2A, protein P3AB, protein P3D (Hepatitis A Virus, Hepatitis A); hepatitis B surface antigen HBsAg, Hepatitis B core antigen HbcAg, polymerase, protein Hbx, preS2 middle surface protein, surface protein L, large S protein, virus protein VP1, virus protein VP2, virus protein VP3, virus protein VP4 (Hepatitis B Virus (HBV), Hepatitis B); envelope glycoprotein E1 gp32 gp35, envelope glycoprotein E2 NS1 gp68 gp70, capsid protein C, core protein Core, polyprotein, virus protein VP1, virus protein VP2, virus protein VP3, virus protein VP4, antigen G, protein NS3, protein NS5A, (Hepatitis C Virus, Hepatitis C); virus protein VP1, virus protein VP2, virus protein VP3, virus protein VP4, large hepatitis delta antigen, small hepatitis delta antigen (Hepatitis D Virus, Hepatitis D); virus protein VP1, virus protein VP2, virus protein VP3, virus protein VP4, capsid protein E2 (Hepatitis E Virus, Hepatitis E); glycoprotein L UL1, uracil-DNA glycosylase UL2, protein UL3, protein UL4, DNA replication protein UL5, portal protein UL6, virion maturation protein UL7, DNA helicase UL8, replication origin-binding protein UL9, glycoprotein M UL10, protein UL11, alkaline exonuclease UL12, serine-threonine protein kinase UL13, tegument protein UL14, terminase UL15, tegument protein UL16, protein UL17, capsid protein VP23 UL18, major capsid protein VP5 UL19, membrane protein UL20, tegument protein UL21, Glycoprotein H (UL22), Thymidine Kinase UL23, protein UL24, protein UL25, capsid protein P40 (UL26, VP24, VP22A), glycoprotein B (UL27), ICP18.5 protein (UL28), major DNA-binding protein ICP8 (UL29), DNA polymerase UL30, nuclear matrix protein UL31, envelope glycoprotein UL32, protein UL33, inner nuclear membrane protein UL34, capsid protein VP26 (UL35), large tegument protein UL36, capsid assembly protein UL37, VP19C protein (UL38), ribonucleotide reductase (Large subunit) UL39, ribonucleotide reductase (Small subunit) UL40, tegument protein/virion host shutoff VHS protein (UL41), DNA polymerase processivity factor UL42, membrane protein UL43, glycoprotein C (UL44), membrane protein UL45, tegument proteins VP11/12 (UL46), tegument protein VP13/14 (UL47), virion maturation protein VP16 (UL48, Alpha-TIF), envelope protein UL49, dUTP diphosphatase UL50, tegument protein UL51, DNA helicase/primase complex protein UL52, glycoprotein K (UL53), transcriptional regulation protein IE63 (ICP27, UL54), protein UL55, protein UL56, viral replication protein ICP22 (IE68, US1), protein US2, serine/threonine-protein kinase US3, glycoprotein G (US4), glycoprotein J (US5), glycoprotein D (US6), glycoprotein I (US7), glycoprotein E (US8), tegument protein US9, capsid/tegument protein US10, Vmw21 protein (US11), ICP47 protein (IE12, US12), major transcriptional activator ICP4 (IE175, RSI), E3 ubiquitin ligase ICPO (IE110), latency-related protein 1 LRP1, latency-related protein 2 LRP2, neurovirulence factor RL1 (ICP34.5), latency-associated transcript LAT (Herpes simplex virus 1 and 2 (HSV-1 and HSV-2), Herpes simplex); heat shock protein Hsp60, cell surface protein HIC, dipeptidyl peptidase type IV DppIV, M antigen, 70 kDa protein, 17 kDa histone-like protein (Histoplasma capsulatum, Histoplasmosis); fatty acid and retinol binding protein-1 FAR-1, tissue inhibitor of metalloproteinase TIMP (TMP), cysteine proteinase ACEY-1, cysteine proteinase ACCP-1, surface antigen Ac-16, secreted protein 2 ASP-2, metalloprotease 1 MTP-1, aspartyl protease inhibitor API-1, surface-associated antigen SAA-1, surface-associated antigen SAA-2, adult-specific secreted factor Xa, serine protease inhibitor anticoagulant AP, cathepsin D-like aspartic protease ARR-1, glutathione S-transferase GST, aspartic protease APR-1, acetylcholinesterase AChE (Ancylostoma duodenale and Necator americanus, Hookworm infection); protein NS1, protein NP1, protein VP1, protein VP2, protein VP3 (Human bocavirus (HBoV), Human bocavirus infection); major surface protein 2 MSP2, major surface protein 4 MSP4, MSP variant SGV1, MSP variant SGV2, outer membrane protein OMP, outer membrande protein 19 OMP-19, major antigenic protein MAPI, major antigenic protein MAP1-2, major antigenic protein MAP1B, major antigenic protein MAP1-3, Erum2510 coding protein, protein GroEL, protein GroES, 30-kDA major outer membrane proteins, GE 100-kDa protein, GE 130-kDa protein, GE 160-kDa protein (Ehrlichia ewingii, Human ewingii ehrlichiosis); major surface proteins 1-5 (MSPla, MSPlb, MSP2, MSP3, MSP4, MSP5), type IV secreotion system proteins VirB2, VirB7, VirBll, VirD4 (Anaplasma phagocytophilum, Human granulocytic anaplasmosis (HGA)); protein NS1, small hydrophobic protein NS2, SH protein, fusion protein F, glycoprotein G, matrix protein M, matrix protein M2-1, matrix protein M2-2, phosphoprotein P, nucleoprotein N, polymerase L (Human metapneumovirus (hMPV), Human metapneumovirus infection); major surface protein 2 MSP2, major surface protein 4 MSP4, MSP variant SGV1, MSP variant SGV2, outer membrane protein OMP, outer membrande protein 19 OMP-19, major antigenic protein MAPI, major antigenic protein MAP1-2, major antigenic protein MAP1B, major antigenic protein MAP1-3, Erum2510 coding protein, protein GroEL, protein GroES, 30-kDA major outer membrane proteins, GE 100-kDa protein, GE 130-kDa protein, GE 160-kDa protein (Ehrlichia chaffeensis, Human monocytic ehrlichiosis); replication protein E1, regulatory protein E2, protein E3, protein E4, protein E5, protein E6, protein E7, protein E8, major capsid protein L1, minor capsid protein L2 (Human papillomavirus (HPV), Human papillomavirus (HPV) infection); fusion protein F, hemagglutinin-neuramidase HN, glycoprotein G, matrix protein M, phosphoprotein P, nucleoprotein N, polymerase L (Human parainfluenza viruses (HPIV), Human parainfluenza virus infection); Hemagglutinin (HA), Neuraminidase (NA), Nucleoprotein (NP), M1 protein, M2 protein, NS1 protein, NS2 protein (NEP protein: nuclear export protein), PA protein, PB1 protein (polymerase basic 1 protein), PB1-F2 protein and PB2 protein (Orthomyxoviridae family, Influenza virus (flu)); genome polyprotein, protein E, protein M, capsid protein C (Japanese encephalitis virus, Japanese encephalitis); RTX toxin, type IV pili, major pilus subunit PilA, regulatory transcription factors PilS and PilR, protein sigma54, outer membrane proteins (Kingella kingae, Kingella kingae infection); prion protein (Kuru prion, Kuru); nucleoprotein N, polymerase L, matrix protein Z, glycoprotein GP (Lassa virus, Lassa fever); peptidoglycan-associated lipoprotein PAL, 60 kDa chaperonin Cpn60 (groEL, HspB), type IV pilin PilE, outer membrane protein MIP, major outer membrane protein MompS, zinc metalloproteinase MSP (Legionella pneumophila, Legionellosis (Legionnaires' disease, Pontiac fever)); P4 nuclease, protein WD, ribonucleotide reductase M2, surface membrane glycoprotein Pg46, cysteine proteinase CP, glucose-regulated protein 78 GRP-78, stage-specific S antigen-like protein A2, ATPase F1, beta-tubulin, heat shock protein 70 Hsp70, KMP-11, glycoprotein GP63, protein BT1, nucleoside hydrolase NH, cell surface protein B1, ribosomal protein P1-like protein PI, sterol 24-c-methy transferase SMT, LACK protein, histone HI, SPB1 protein, thiol specific antioxidant TSA, protein antigen STI1, signal peptidase SP, histone H2B, surface antigen PSA-2, cystein proteinase b Cpb (Leishmania genus, Leishmaniasis); major membrane protein I, serine-rich antigen-45 kDa, 10 kDa caperonin GroES, HSP kDa antigen, amino-oxononanoate synthase AONS, protein recombinase A RecA, Acetyl-/propionyl-coenzyme A carboxylase alpha, alanine racemase, 60 kDa chaperonin 2, ESAT-6-like protein EcxB (L-ESAT-6), protein Lsr2, protein ML0276, Heparin-binding hemagglutinin HBHA, heat-shock protein 65 Hsp65, mycPl or ML0041 coding protein, htrA2 or ML0176 coding protein, htrA4 or ML2659 coding protein, gcp or ML0379 coding protein, clpC or ML0235 coding protein (Mycobacterium leprae and Mycobacterium lepromatosis, Leprosy); outer membrane protein LipL32, membrane protein LIC10258, membrane protein LP30, membrane protein LIC12238, Ompa-like protein Lsa66, surface protein LigA, surface protein LigB, major outer membrane protein OmpL1, outer membrane protein LipL41, protein LigAni, surface protein LcpA, adhesion protein LipL53, outer membrane protein UpL32, surface protein Lsa63, flagellin FlaB1, membrane lipoprotein LipL21, membrane protein pL40, leptospiral surface adhesin Lsa27, outer membrane protein OmpL36, outer membrane protein OmpL37, outer membrane protein OmpL47, outer membrane protein OmpL54, acyltransferase LpxA (Leptospira genus, Leptospirosis); listeriolysin O precursor Hly (LLO), invasion-associated protein lap (P60), Listeriolysin regulatory protein PrfA, Zinc metalloproteinase Mpl, Phosphatidylinositol-specific phospholipase C PLC (PlcA, PlcB), 0-acetyltransferase Oat, ABC-transporter permease Im.G_1771, adhesion protein LAP, LAP receptor Hsp60, adhesin LapB, haemolysin listeriolysin OLLO, protein ActA, Internalin A InIA, protein InIB (Listeria monocytogenes, Listeriosis); outer surface protein A OspA, outer surface protein OspB, outer surface protein OspC, decorin binding protein A DbpA, decorin binding protein B DbpB, flagellar filament 41 kDa core protein Fla, basic membrane protein A BmpA (Immunodominant antigen P39), outer surface 22 kDa lipoprotein precursor (antigen IPLA7), variable surface lipoprotein vlsE (usually Borrelia burgdorferi and other Borrelia species, Lyme disease (Lyme borreliosis)); venom allergen homolog-like protein VAL-1, abundant larval transcript ALT-1, abundant larval transcript ALT-2, thioredoxin peroxidase TPX, vespid allergen homologue VAH, thiordoxin peroxidase 2 TPX-2, antigenic protein SXP (peptides N, N1, N2, and N3), activation associated protein-1 ASP-1, thioredoxin TRX, transglutaminase BmTGA, glutathione-S-transferases GST, myosin, vespid allergen homologue VAH, 175 kDa collagenase, glyceraldehyde-3-phosphate dehydrogenase GAPDH, cuticular collagen Col-4, Secreted Larval Acidic Proteins SLAPs, chitinase CHI-1, maltose binding protein MBP, glycolytic enzyme fructose-1,6-bisphosphate aldolase Fba, tropomyosin TMY-1, nematode specific gene product OvB20, onchocystatin CPI-2, protein Cox-2 (Wuchereria bancrofti and Brugia malayi, Lymphatic filariasis (Elephantiasis)); glycoprotein GP, matrix protein Z, polymerase L, nucleoprotein N (Lymphocytic choriomeningitis virus (LCMV), Lymphocytic choriomeningitis); thrombospondin-related anonymous protein TRAP, SSP2 Sporozoite surface protein 2, apical membrane antigen 1 AMA1, rhoptry membrane antigen RMA1, acidic basic repeat antigen ABRA, cell-traversal protein PF, protein Pvs25, merozoite surface protein 1 MSP-1, merozoite surface protein 2 MSP-2, ring-infected erythrocyte surface antigen RESALiver stage antigen 3 LSA-3, protein Eba-175, serine repeat antigen 5 SERA-5, circumsporozoite protein CS, merozoite surface protein 3 MSP3, merozoite surface protein 8 MSP8, enolase PF10, hepatocyte erythrocyte protein 17 kDa HEP17, erythrocyte membrane protein 1 EMP1, protein Kbeta merozoite surface protein 4/5 MSP 4/5, heat shock protein Hsp90, glutamate-rich protein GLURP, merozoite surface protein 4 MSP-4, protein STARP, circumsporozoite protein-related antigen precursor CRA (Plasmodium genus, Malaria); nucleoprotein N, membrane-associated protein VP24, minor nucleoprotein VP30, polymerase cofactor VP35, polymerase L, matrix protein VP40, envelope glycoprotein GP (Marburg virus, Marburg hemorrhagic fever (MHF)); protein C, matrix protein M, phosphoprotein P, non-structural protein V, hemagglutinin glycoprotein H, polymerase L, nucleoprotein N, fusion protein F (Measles virus, Measles); members of the ABC transporter family (LolC, OppA, and PotF), putative lipoprotein releasing system transmembrane protein LolC/E, fiagellin FliC, Burkholderia intracellular motility A BimA, bacterial Elongation factor-Tu EF-Tu, 17 kDa OmpA-like protein, boaA coding protein, boaB coding protein (Burkholderia pseudomallei, Melioidosis (Whitmore's disease)); pilin proteins, minor pilin-associated subunit pilC, major pilin subunit and variants pilE, pilS, phase variation protein porA, Porin B PorB, protein TraD, Neisserial outer membrane antigen H.8, 70 kDa antigen, major outer membrane protein PI, outer membrane proteins PIA and PIB, W antigen, surface protein A NspA, transferrin binding protein TbpA, transferrin binding protein TbpB, PBP2, mtrR coding protein, ponA coding protein, membrane permease FbpBC, FbpABC protein system, LbpAB proteins, outer membrane protein Opa, outer membrane transporter FetA, iron-repressed regulator MpeR, factor H-binding protein fHbp, adhesin NadA, protein NhbA, repressor FarR (Neisseria meningitidis, Meningococcal disease); 66 kDa protein, 22 kDa protein (usually Metagonimus yokagawai, Metagonimiasis); polar tube proteins (34, 75, and 170 kDa in Glugea, 35, 55 and 150 kDa in Encephalitozoon), kinesin-related protein, RNA polymerase II largest subunit, similar to integral membrane protein YIPA, anti-silencing protein 1, heat shock transcription factor HSF, protein kinase, thymidine kinase, NOP-2 like nucleolar protein (Microsporidia phylum, Microsporidiosis); CASP8 and FADD-like apoptosis regulator, Glutathione peroxidase GPX1, RNA helicase NPH-II NPH2, Poly(A) polymerase catalytic subunit PAPL, Major envelope protein P43K, early transcription factor 70 kDa subunit VETFS, early transcription factor 82 kDa subunit VETFL, metalloendopeptidase G1-type, nucleoside triphosphatase I NPH1, replication protein A28-like MC134L, RNA polymerase 7 kDa subunit RP07 (Molluscum contagiosum virus (MCV), Molluscum contagiosum (MC)); matrix protein M, phosphoprotein PN, small hydrophobic protein SH, nucleoprotein N, protein V, fusion glycoprotein F, hemagglutinin-neuraminidase HN, RNA polymerase L (Mumps virus, Mumps); Outer membrane proteins OM, cell surface antigen OmpA, cell surface antigen OmpB (sca5), cell surface protein SCA4, cell surface protein SCA1, intracytoplasmic protein D, crystalline surface layer protein SLP, protective surface protein antigen SPA (Rickettsia typhi, Murine typhus (Endemic typhus)); adhesin PI, adhesion P30, protein p116, protein P40, cytoskeletal protein HMW1, cytoskeletal protein HMW2, cytoskeletal protein HMW3, MPN152 coding protein, MPN426 coding protein, MPN456 coding protein, MPN-500 coding protein (Mycoplasma pneumoniae, Mycoplasma pneumonia); NocA, Iron dependent regulatory protein, VapA, VapD, VapF, VapG, caseinolytic protease, filament tip-associated 43-kDa protein, protein P24, protein P61, 15-kDa protein, 56-kDa protein (usually Nocardia asteroides and other Nocardia species, Nocardiosis); venom allergen homolog-like protein VAL-1, abundant larval transcript ALT-1, abundant larval transcript ALT-2, thioredoxin peroxidase TPX, vespid allergen homologue VAH, thiordoxin peroxidase 2 TPX-2, antigenic protein SXP (peptides N, N1, N2, and N3), activation associated protein-1 ASP-1, Thioredoxin TRX, transglutaminase BmTGA, glutathione-S-transferases GST, myosin, vespid allergen homologue VAH, 175 kDa collagenase, glyceraldehyde-3-phosphate dehydrogenase GAPDH, cuticular collagen Col-4, Secreted Larval Acidic Proteins SLAPs, chitinase CHI-1, maltose binding protein MBP, glycolytic enzyme fructose-1,6-bisphosphate aldolase Fba, tropomyosin TMY-1, nematode specific gene product OvB20, onchocystatin CPI-2, Cox-2 (Onchocerca volvulus, Onchocerciasis (River blindness)); 43 kDa secreted glycoprotein, glycoprotein gpO, glycoprotein gp75, antigen Pb27, antigen Pb40, heat shock protein Hsp65, heat shock protein Hsp70, heat shock protein Hsp90, protein P10, triosephosphate isomerase TPI, N-acetyl-glucosamine-binding lectin Paracoccin, 28 kDa protein Pb28 (Paracoccidioides brasiliensis, Paracoccidioidomycosis (South American blastomycosis)); 28-kDa cruzipain-like cystein protease Pw28CCP (usually Paragonimus westermani and other Paragonimus species, Paragonimiasis); outer membrane protein OmpH, outer membrane protein Omp28, protein PM1539, protein PM0355, protein PM1417, repair protein MutL, protein BcbC, protein PM0305, formate dehydrogenase-N, protein PM0698, protein PM1422, DNA gyrase, lipoprotein PlpE, adhesive protein Cp39, heme acquisition system receptor HasR, 39 kDa capsular protein, iron-regulated OMP IROMP, outer membrane protein OmpA87, fimbrial protein Ptf, fimbrial subunit protein PtfA, transferrin binding protein Tbp1, esterase enzyme MesA, Pasteurella multocida toxin PMT, adhesive protein Cp39 (Pasteurella genus, Pasteurellosis); “filamentous hemagglutinin FhaB, adenylate cyclase CyaA, pertussis toxin subunit 4 precursor PtxD, pertactin precursor Pm, toxin subunit 1 PtxA, protein Cpn60, protein brkA, pertussis toxin subunit 2 precursor PtxB, pertussis toxin subunit 3 precursor PtxC, pertussis toxin subunit 5 precursor PtxE, pertactin Pm, protein Fim2, protein Fim3; “(Bordetella pertussis, Pertussis (Whooping cough)); “F1 capsule antigen, virulence-associated V antigen, secreted effector protein LcrV, V antigen, outer membrane protease Pla, secreted effector protein YopD, putative secreted protein-tyrosine phosphatase YopH, needle complex major subunit YscF, protein kinase YopO, putative autotransporter protein YapF, inner membrane ABC-transporter YbtQ (Irp7), putative sugar binding protein YPO0612, heat shock protein 90 HtpG, putative sulfatase protein YdeN, outer-membrane lipoprotein carrier protein LolA, secretion chaperone YerA, putative lipoprotein YPO0420, hemolysin activator protein HpmB, pesticin/yersiniabactin outer membrane receptor Psn, secreted effector protein YopE, secreted effector protein YopF, secreted effector protein YopK, outer membrane protein YopN, outer membrane protein YopM, Coagulase/fibrinolysin precursor Pla; “(Yersinia pestis, Plague); protein PhpA, surface adhesin PsaA, pneumolysin Ply, ATP-dependent protease CIp, lipoate-protein ligase LplA, cell wall surface anchored protein psrP, sortase SrtA, glutamyl-tRNA synthetase GltX, choline binding protein A CbpA, pneumococcal surface protein A PspA, pneumococcal surface protein C PspC, 6-phosphogluconate dehydrogenase Gnd, iron-binding protein PiaA, Murein hydrolase LytB, proteon LytC, protease A1 (Streptococcus pneumoniae, Pneumococcal infection); major surface protein B, kexin-like protease KEX1, protein A12, 55 kDa antigen P55, major surface glycoprotein Msg (Pneumocystis jirovecii, Pneumocystis pneumonia (PCP)); genome polyprotein, polymerase 3D, viral capsid protein VP1, viral capsid protein VP2, viral capsid protein VP3, viral capsid protein VP4, protease 2A, protease 3C (Poliovirus, Poliomyelitis); protein Nfal, exendin-3, secretory lipase, cathepsin B-like protease, cysteine protease, cathepsin, peroxiredoxin, protein CrylAc (usually Naegleria fowleri, Primary amoebic meningoencephalitis (PAM)); agnoprotein, large T antigen, small T antigen, major capsid protein VP1, minor capsid protein Vp2 (JC virus, Progressive multifocal leukoencephalopathy); low calcium response protein E LCrE, chlamydial outer protein N CopN, serine/threonine-protein kinase PknD, acyl-carrier-protein S-malonyltransferase FabD, single-stranded DNA-binding protein Ssb, major outer membrane protein MOMP, outer membrane protein 2 Omp2, polymorphic membrane protein family (Pmp1, Pmp2, Pmp3, Pmp4, Pmp5, Pmp6, Pmp7, Pmp8, Pmp9, Pmp10, Pmp11, Pmp12, Pmp13, Pmp14, Pmp15, Pmp16, Pmp17, Pmp18, Pmp19, Pmp20, Pmp21) (Chlamydophila psittaci, Psittacosis); outer membrane protein PI, heat shock protein B HspB, peptide ABC transporter, GTP-binding protein, protein IcmB, ribonuclease R, phosphatas SixA, protein DsbD, outer membrane protein TolC, DNA-binding protein PhoB, ATPase DotB, heat shock protein B HspB, membrane protein Coml, 28 kDa protein, DNA-3-methyladenine glycosidase I, pouter membrane protein OmpH, outer membrane protein AdaA, glycine cleavage system T-protein (Coxiella burnetii, Q fever); nucleoprotein N, large structural protein L, phophoprotein P, matrix protein M, glycoprotein G (Rabies virus, Rabies); fusion protein F, nucleoprotein N, matrix protein M, matrix protein M2-1, matrix protein M2-2, phophoprotein P, small hydrophobic protein SH, major surface glycoprotein G, polymerase L, non-structural protein 1 NS1, non-structural protein 2 NS2 (Respiratory syncytial virus (RSV), Respiratory syncytial virus infection); genome polyprotein, polymerase 3D, viral capsid protein VP1, viral capsid protein VP2, viral capsid protein VP3, viral capsid protein VP4, protease 2A, protease 3C (Rhinovirus, Rhinovirus infection); outer membrane proteins OM, cell surface antigen OmpA, cell surface antigen OmpB (sca5), cell surface protein SCA4, cell surface protein SCA1, protein PS120, intracytoplasmic protein D, protective surface protein antigen SPA (Rickettsia genus, Rickettsial infection); outer membrane proteins OM, cell surface antigen OmpA, cell surface antigen OmpB (sca5), cell surface protein SCA4, cell surface protein SCA1, intracytoplasmic protein D (Rickettsia akari, Rickettsialpox); envelope glycoprotein GP, polymerase L, nucleoprotein N, non-structural protein NSS (Rift Valley fever virus, Rift Valley fever (RVF)); outer membrane proteins OM, cell surface antigen OmpA, cell surface antigen OmpB (sca5), cell surface protein SCA4, cell surface protein SCA1, intracytoplasmic protein D (Rickettsia rickettsii, Rocky mountain spotted fever (RMSF)); non-structural protein 6 NS6, non-structural protein 2 NS2, intermediate capsid protein VP6, inner capsid protein VP2, non-structural protein 3 NS3, RNA-directed RNA polymerase L, protein VP3, non-structural protein 1 NS1, non-structural protein 5 NS5, outer capsid glycoprotein VP7, nonstructural glycoprotein 4 NS4, outer capsid protein VP4; (Rotavirus, Rotavirus infection); polyprotein P200, glycoprotein E1, glycoprotein E2, protein NS2, capsid protein C (Rubella virus, Rubella); chaperonin GroEL (MopA), inositol phosphate phosphatase SopB, heat shock protein HsIU, chaperone protein DnaJ, protein TviB, protein IroN, flagellin FliC, invasion protein SipC, glycoprotein gp43, outer membrane protein LamB, outer membrane protein PagC, outer membrane protein TolC, outer membrane protein NmpC, outer membrane protein FadL, transport protein SadA, transferase WgaP, effector proteins SifA, SteC, SseL, SseJ and SseF (Salmonella genus, Salmonellosis); “protein 14, non-structural protein NS7b, non-structural protein NS8a, protein 9b, protein 3a, nucleoprotein N, non-structural protein NS3b, non-structural protein NS6, protein 7a, non-structural protein NS8b, membrane protein M, envelope small membrane protein EsM, replicase polyprotein 1a, spike glycoprotein S, replicase polyprotein lab; SARS coronavirus, SARS (Severe Acute Respiratory Syndrome)); serin protease, Atypical Sarcoptes Antigen 1 ASA1, glutathione S-transferases GST, cystein protease, serine protease, apolipoprotein (Sarcoptes scabiei, Scabies); glutathione S-transferases GST, paramyosin, hemoglbinase SM32, major egg antigen, 14 kDa fatty acid-binding protein Sm14, major larval surface antigen P37, 22.6 kDa tegumental antigen, calpain CANP, triphosphate isomerase Tim, surface protein 9B, outer capsid protein VP2, 23 kDa integral membrane protein Sm23, Cu/Zn-superoxide dismutase, glycoprotein Gp, myosin (Schistosoma genus, Schistosomiasis (Bilharziosis)); 60 kDa chaperonin, 56 kDa type-specific antigen, pyruvate phosphate dikinase, 4-hydroxybenzoate octaprenyltransferase (Orientia tsutsugamushi, Scrub typhus); dehydrogenase GuaB, invasion protein Spa32, invasin IpaA, invasin IpaB, invasin IpaC, invasin IpaD, invasin IpaH, invasin IpaJ (Shigella genus, Shigellosis (Bacillary dysentery)); protein P53, virion protein US10 homolog, transcriptional regulator IE63, transcriptional transactivator IE62, protease P33, alpha trans-inducing factor 74 kDa protein, deoxyuridine 5′-triphosphate nucleotidohydrolase, transcriptional transactivator IE4, membrane protein UL43 homolog, nuclear phosphoprotein UL3 homolog, nuclear protein UL4 homolog, replication origin-binding protein, membrane protein 2, phosphoprotein 32, protein 57,DNA polymerase processivity factor, portal protein 54, DNA primase, tegument protein UL14 homolog, tegument protein UL21 homolog, tegument protein UL55 homolog, tripartite terminase subunit UL33 homolog, tri partite terminase subunit UL15 homolog, capsid-binding protein 44, virion-packaging protein 43 (Varicella zoster virus (VZV), Shingles (Herpes zoster)); truncated 3-beta hydroxy-5-ene steroid dehydrogenase homolog, virion membrane protein A13, protein A19, protein A31, truncated protein A35 homolog, protein A37.5 homolog, protein A47, protein A49, protein A51, semaphorin-like protein A43, serine proteinase inhibitor 1, serine proteinase inhibitor 2, serine proteinase inhibitor 3, protein A6, protein B15, protein CI, protein C5, protein C6, protein F7, protein F8, protein F9, protein F11, protein F14, protein F15, protein F16 (Variola major or Variola minor, Smallpox (Variola)); adhesin/glycoprotein gp70, proteases (Sporothrix schenckii, Sporotrichosis); heme-iron binding protein IsdB, collagen adhesin Cna, clumping factor A ClfA, protein MecA, fibronectin-binding protein A FnbA, enterotoxin type A EntA, enterotoxin type B EntB, enterotoxin type C EntC1, enterotoxin type C EntC2, enterotoxin type D EntD, enterotoxin type E EntE, Toxic shock syndrome toxin-1 TSST-1, Staphylokinase, Penicillin binding protein 2a PBP2a (MecA), secretory antigen SssA (Staphylococcus genus, Staphylococcal food poisoning); heme-iron binding protein IsdB, collagen adhesin Cna, clumping factor A ClfA, protein MecA, fibronectin-binding protein A FnbA, enterotoxin type A EntA, enterotoxin type B EntB, enterotoxin type C EntC1, enterotoxin type C EntC2, enterotoxin type D EntD, enterotoxin type E EntE, Toxic shock syndrome toxin-1 TSST-1, Staphylokinase, Penicillin binding protein 2a PBP2a (MecA), secretory antigen SssA (Staphylococcus genus e.g. aureus, Staphylococcal infection); antigen Ss-IR, antigen NIE, strongylastacin, Na+-K+ ATPase Sseat-6, tropomysin SsTmy-1, protein LEC-5, 41 kDa antigen P5, 41-kDa larval protein, 31-kDa larval protein, 28-kDa larval protein (Strongyloides stercoralis, Strongyloidiasis); glycerophosphodiester phosphodiesterase GlpQ (Gpd), outer membrane protein TmpB, protein Tp92, antigen TpFl, repeat protein Tpr, repeat protein F TprF, repeat protein G TprG, repeat protein I Tprl, repeat protein J TprJ, repeat protein K TprK, treponemal membrane protein A TmpA, lipoprotein, 15 kDa Tppl5, 47 kDa membrane antigen, miniferritin TpFl, adhesin Tp0751, lipoprotein TP0136, protein TpN17, protein TpN47, outer membrane protein TP0136, outer membrane protein TP0155, outer membrane protein TP0326, outer membrane protein TP0483, outer membrane protein TP0956 (Treponema pallidum, Syphilis); Cathepsin L-like proteases, 53/25-kDa antigen, 8 kDa family members, cysticercus protein with a marginal trypsin-like activity TsAg5, oncosphere protein TSOL18, oncosphere protein TSOL45-1A, lactate dehydrogenase A LDHA, lactate dehydrogenase B LDHB (Taenia genus, Taeniasis); tetanus toxin TetX, tetanus toxin C TTC, 140 kDa S layer protein, flavoprotein beta-subunit CT3, phospholipase (lecithinase), phosphocarrier protein HPr (Clostridium tetani, Tetanus (Lockjaw)); genome polyprotein, protein E, protein M, capsid protein C (Tick-borne encephalitis virus (TBEV), Tick-borne encephalitis); 58-kDa antigen, 68-kDa antigens, Toxocara larvae excretory-secretory antigen TES, 32-kDa glycoprotein, glycoprotein TES-70, glycoprotein GP31, excretory-secretory antigen TcES-57, perienteric fluid antigen Pe, soluble extract antigens Ex, excretory/secretory larval antigens ES, antigen TES-120, polyprotein allergen TBA-1, cathepsin L-like cysteine protease c-cpl-1, 26-kDa protein (Toxocara canis or Toxocara cati, Toxocariasis (Ocular Larva Migrans (OLM) and Visceral Larva Migrans (VLM))); microneme proteins (MIC1, MIC2, MIC3, MIC4, MIC5, MIC6, MIC7, MIC8), rhoptry protein Rop2, rhoptry proteins (Rop1, Rop2, Rop3, Rop4, Rop5, Rop6, Rop7, Rop16, Rjop17), protein SR1, surface antigen P22, major antigen p24, major surface antigen p30, dense granule proteins (GRA1, GRA2, GRA3, GRA4, GRA5, GRA6, GRA7, GRA8, GRA9, GRA10), 28 kDa antigen, surface antigen SAG1, SAG2 related antigen, nucleoside-triphosphatase 1, nucleoside-triphosphatase 2, protein Stt3, HesB-like domain-containing protein, rhomboid-like protease 5, toxomepsin 1 (Toxoplasma gondii, Toxoplasmosis); 43 kDa secreted glycoprotein, 53 kDa secreted glycoprotein, paramyosin, antigen Ts21, antigen Ts87, antigen p46000, TSL-1 antigens, caveolin-1 CAV-1, 49 kDa newborn larva antigen, prosaposin homologue, serine protease, serine proteinase inhibitor, 45-kDa glycoprotein Gp45 (Trichinella spiralis, Trichinellosis); Myb-like transcriptional factors (Myb1, Myb2, Myb3), adhesion protein AP23, adhesion protein AP33, adhesin protein AP33-3, adhesins AP51, adhesin AP65, adhesion protein AP65-1, alpha-actinin, kinesin-associated protein, teneurin, 62 kDa proteinase, subtilisin-like serine protease SUB1, cysteine proteinase gene 3 CP3, alpha-enolase Enol, cysteine proteinase CP30, heat shock proteins (Hsp70, Hsp60), immunogenic protein P270, (Trichomonas vaginalis, Trichomoniasis); beta-tubulin, 47-kDa protein, secretory leucocyte-like proteinase-1 SLP-1, 50-kDa protein TT50, 17 kDa antigen, 43/47 kDa protein (Trichuris trichiura, Trichuriasis (Whipworm infection)); protein ESAT-6 (EsxA), 10 kDa filtrate antigen EsxB, secreted antigen 85-B FBPB, fibronectin-binding protein A FbpA (Ag85A), serine protease PepA, PPE family protein PPE18, fibronectin-binding protein D FbpD, immunogenic protein MPT64, secreted protein MPT51, catalase-peroxidase-peroxynitritase T KATG, periplasmic phosphate-binding lipoprotein PSTS3 (PBP-3, Phos-1), iron-regulated heparin binding hemagglutinin Hbha, PPE family protein PPE14, PPE family protein PPE68, protein Mtb72F, protein Apa, immunogenic protein MPT63, periplasmic phosphate-binding lipoprotein PSTS1 (PBP-1), molecular chaperone DnaK, cell surface lipoprotein Mpt83, lipoprotein P23, phosphate transport system permease protein pstA, 14 kDa antigen, fibronectin-binding protein C FbpC1, Alanine dehydrogenase TB43, Glutamine synthetase 1, ESX-1 protein, protein CFP10, TB10.4 protein, protein MPT83, protein MTB12, protein MTB8, Rpf-like proteins, protein MTB32, protein MTB39, crystallin, heat-shock protein HSP65, protein PST-S(usually Mycobacterium tuberculosis, Tuberculosis); outer membrane protein FobA, outer membrane protein FobB, intracellular growth locus IglC1, intracellular growth locus IglC2, aminotransferase Wbt1, chaperonin GroEL, 17 kDa major membrane protein TUL4, lipoprotein LpnA, chitinase family 18 protein, isocitrate dehydrogenase, Nif3 family protein, type IV pili glycosylation protein, outer membrane protein tolC, FAD binding family protein, type IV pilin multimeric outer membrane protein, two component sensor protein KdpD, chaperone protein DnaK, protein TolQ (Francisella tularensis, Tularemia); “MB antigen, urease, protein GyrA, protein GyrB, protein ParC, protein ParE, lipid associated membrane proteins LAMP, thymidine kinase TK, phospholipase PL-A1, phospholipase PL-A2, phospholipase PL-C, surface-expressed 96-kDa antigen; (Ureaplasma urealyticum, Ureaplasma urealyticum infection); non-structural polyprotein, structural polyprotein, capsid protein CP, protein E1, protein E2, protein E3, protease PI, protease P2, protease P3 (Venezuelan equine encephalitis virus, Venezuelan equine encephalitis); glycoprotein GP, matrix protein Z, polymerase L, nucleoprotein N (Guanarito virus, Venezuelan hemorrhagic fever); polyprotein, protein E, protein M, capsid protein C, protease NS3, protein NS1, protein NS2A, protein AS2B, protein NS4A, protein NS4B, protein NS5 (West Nile virus, West Nile Fever); capsid protein CP, protein E1, protein E2, protein E3, protease P2 (Western equine encephalitis virus, Western equine encephalitis); genome polyprotein, protein E, protein M, capsid protein C, protease NS3, protein NS1, protein NS2A, protein AS2B, protein NS4A, protein NS4B, protein NS5 (Yellow fever virus, Yellow fever); putative Yop targeting protein YobB, effector protein YopD, effector protein YopE, protein YopH, effector protein YopJ, protein translocation protein YopK, effector protein YopT, protein YpkA, flagellar biosyntheses protein FlhA, peptidase M48, potassium efflux system KefA, transcriptional regulator RovA, adhesin Ifp, translocator protein LcrV, protein PcrV, invasin Inv, outer membrane protein OmpF-like porin, adhesin YadA, protein kinase C, phospholipase CI, protein PsaA, mannosyltransferase-like protein WbyK, protein YscU, antigen YPMa (Yersinia pseudotuberculosis, Yersinia pseudotuberculosis infection); effector protein YopB, 60 kDa chaperonin, protein WbcP, tyrosin-protein phosphatase YopH, protein YopQ, enterotoxin, Galactoside permease, reductase NrdE, protein YasN, Invasin Inv, adhesin YadA, outer membrane porin F OmpF, protein UspA1, protein EibA, protein Hia, cell surface protein Ail, chaperone SycD, protein LcrD, protein LcrG, protein LcrV, protein SycE, protein YopE, regulator protein TyeA, protein YopM, protein YopN, protein YopO, protein YopT, protein YopD, protease ClpP, protein MyfA, protein FilA, and protein PsaA (Yersinia enterocolitica, Yersiniosis).

In one example, the antigen is a viral antigen. For example, the viral antigen is from a respiratory virus. In one example, the respiratory virus is selected from the group consisting of influenza virus, respiratory syncytial virus, parainfluenza viruses, metapneumovirus, rhinovirus, coronaviruses, adenoviruses and bocaviruses.

In one example, the viral antigen is from an influenza virus. In one example, the viral antigen is from a respiratory syncytial virus. In one example, the viral antigen is from a parainfluenza virus. In one example, the viral antigen is from a metapneumovirus.

In one example, the viral antigen is from a rhinovirus. In one example, the viral antigen is from a coronavirus. In one example, the viral antigen is from an adenovirus. In one example, the viral antigen is from a bocavirus. In one example, the antigens are viral antigens from an influenza virus and/or a coronavirus. In one example, the antigens are viral antigens from a betacoronavirus.

In examples wherein the infectious disease is influenza, the antigens include hemagglutinin (HA), neuraminidase (NA), nucleoprotein (NP), matrix protein 1 (M1), matrix protein 2 (M2), non-structural protein 1 (NS1), non-structural protein 2 (NS2), nuclear export protein (NEP), polymerase acidic protein (PA), polymerase basic protein PB1, PB1-F2, or polymerase basic protein 2 (PB2) of an influenza virus or a fragment or variant thereof. In one example, the antigen is a peptide or protein derived from hemagglutinin (HA) and/or neuraminidase (NA) of an influenza virus or a fragment or variant thereof. The HA and/or NA may, independently, be derived from an influenza A virus or an influenza B virus or a fragment of either

In one example, the antigens are from an influenza A virus strain. For example, the antigens are an influenza A virus hemagglutinin (HA) protein, a neuraminidase (NA) protein, a matrix (M) protein, a nucleoprotein (NP), a non-structural (NS) protein, or an immunogenic fragment or variant thereof. In one example, the antigens are an influenza A hemagglutinin (HA) subtype H1, H2, H3, H4, H5, H6, H7, H8, H9, H10, H11, H12, H13, H14, H15 or H16 and/or an influenza A neuraminidase (NA) subtype N1, N2, N3, N4, N5, N6, N7, N8 or N9 and/or an influenza A matrix (M) protein subtype M1 or M2 and/or an influenza A non-structural (NS) protein subtype NS1 or NS2. In one example, the antigens are a H5 hemagglutinin protein and/or a N1 neuraminidase protein. In one example, the antigen is an influenza A virus M protein and/or a NP. For example, the antigen is a M1 matrix protein and/or a NP protein. In one example, the NP protein is an A/California/07/09 strain. In one example, the antigen is an influenza A virus HA protein, a NA protein and/or a M protein. For example, the antigens are a H5 hemagglutinin protein and/or a N1 neuraminidase protein and/or a M1 matrix protein and/or a M2 matrix protein.

In examples wherein the infectious disease is caused by coronavirus (e.g. SARS-CoV1 and/or SARS-CoV2), the antigen is a peptide or protein derived from Spike (S) protein or nucleocapsid (N). The S and/or N may, independently, be derived from a variant of SARS-CoV-2 (e.g. the original strain, alpha, delta, omicron) or a fragment of either. In one example, the antigens are a SARS-CoV-2 N protein and/or a S protein from SARS-CoV-2 strain 2019-nCoV/USA-WA1/2020.

In one example, an immune response induced by a composition described herein has been established in an appropriate model system. In one example, a protective response against infection (e.g. SARS-CoV-2 or influenza) induced by a composition described herein has been established in an appropriate model system. For example, such a response may have been demonstrated in an animal model, e.g., a non-human primate model (e.g., rhesus macaques) and/or a mouse model.

### Screening Assays

Assays may be conducted to assess the efficiency and efficacy of the lipid nanoparticle compositions described herein including, for example, serology and immune responses. Suitable methods are available to those skilled in the art and include, but are not limited to, antigen expression, Microneutralization Assay and Antigen Specific T cell Responses.

### Antigen Expression

In one example, the lipid nanoparticle composition is assessed for expression of the gene of interest.

For example, antigen expression is detected using antibodies against the gene of interest. In one example, the number of cells positive for antigen expression is measured by e.g., fluorescence-activated cell sorting (FACS). In another example the mean fluorescence intensity (MFI) is determined using e.g., FACS. In a further example, the specific potency value or the probability of successful transfection per unit mass of RNA is calculated.

### Microneutralization Assay

In one example, the lipid nanoparticle composition is assessed for antibody responses. For example, the lipid nanoparticle composition is assessed using a microneutralisation assay. Methods of performing a microneutralization assay will be apparent to the skilled person. In one example, the microneutralization assay is a short form assay. For one example, a virus fluorescent focus-based microneutralization assay is performed. In another example, the microneutralization assay is a long form assay.

Antigen Specific T cell Responses

In one example, the lipid nanoparticle composition is assessed for its ability to induce antigen specific T cell responses. Methods of assessing induction of antigen specific T cell responses will be apparent to the skilled person and/or are described herein.

For example, antigen-specific T cell detection is performed on splenic cultures. Briefly, splenocyte cultures are established in T cell medium and cell cultures are either stimulated with antigenic peptides or unstimulated. In one example, antigen-specific T cell responses are determined using flow cytometry.

### Kits

Another example of the disclosure provides kits containing a composition containing an enriched population of LNP produced using the methods described herein which is useful for the treatment or prevention or delaying progression of a disease or disorder as described above.

In one example, the kit comprises (a) a container comprising a composition containing an enriched population of LNP and/or a pharmaceutically acceptable carrier or diluent; and (b) a package insert with instructions for treating or preventing or delaying progression of a disease or disorder (e.g., COVID-19 or ARDS) in a subject.

In accordance with this example of the disclosure, the package insert is on or associated with the container. Suitable containers include, for example, bottles, vials, syringes, etc. The containers may be formed from a variety of materials such as glass or plastic. The container holds or contains a composition that is effective for a disease or disorder of the disclosure and may have a sterile access port (for example, the container may be an intravenous solution bag or a vial having a stopper pierceable by a hypodermic injection needle). At least one active agent in the composition is the self-replicating RNA. The label or package insert indicates that the composition is used for treating a subject eligible for treatment, e.g., one having or predisposed to developing influenza, an influenza virus infection, a SARS-CoV-2 infection, COVID-19 and/or ARDS, with specific guidance regarding dosing amounts and intervals of treatment and any other medicament being provided. The kit may further comprise an additional container comprising a pharmaceutically acceptable diluent buffer, such as bacteriostatic water for injection (BWFI), phosphate-buffered saline, Ringer's solution, and/or dextrose solution. The kit may further include other materials desirable from a commercial and user standpoint, including other buffers, diluents, filters, needles, and syringes.

The invention is further disclosed in the following numbered paragraphs:

1. A method of preparing an enriched population of lipid nanoparticles (LNPs), comprising the steps of:
contacting a composition comprising lipid nanoparticles and RNA with an anion exchanger under conditions such that the anion exchanger binds unencapsulated RNA;
collecting the effluent to obtain the enriched population of lipid nanoparticles.

2. The method of paragraph 1, wherein the unencapsulated RNA comprises free RNA, RNA that is associated with the surface of the lipid nanoparticles and/or partially encapsulated RNA.

3. The method of paragraph 1 or paragraph 2, wherein the composition comprising lipid nanoparticles has an ionic concentration of between 5 and 50 mM.

4. The method of any one of paragraphs 1 to 3, wherein the composition comprising lipid nanoparticles and RNA comprises salt at a concentration of not greater than 100 mM.

5. The method of any one of paragraphs 1 to 4, wherein the composition comprising lipid nanoparticles has conductivity of less than 15 mS/cm.

6. The method of any one of paragraphs 1 to 5, wherein the composition comprising lipid nanoparticles further comprises a buffer selected from citrate buffer, bis-tris, histidine, acetate buffer, phosphate buffer, tris buffer and/or combinations thereof.

7. The method of paragraph 6, wherein the buffer is a bis-tris buffer, histidine buffer or a Tris buffer.

8. The method of any one of paragraphs 1 to 7, wherein the LNP comprises an ionizable lipid and one or more of a neutral lipid, a PEGylated lipid, and a structural lipid.

9. The method of paragraph 8, wherein the ionisable lipid is selected from the group consisting of 3-(didodecylamino)-N1,N1,4-tridodecyl-1-piperazineethanamine (KL10),
N1-[2-(didodecylamino)ethyl]-N1,N4,N4-tridodecyl-1,4-piperazinediethanamine (KL22),
14,25-ditridecyl-15,18,21,24-tetraaza-octatriacontane (KL25),
1,2-dilinoleyloxy-N,N-dimethylaminopropane (DLin-DMA),
2,2-dilinoleyl-4-dimethylaminomethyl-[1,3]-dioxolane (DLin-K-DMA),
1,2-dioleoyl-3-trimethylammonium propane (DOTAP),
1,2-distearyloxy-N,N-dimethyl-3-aminopropane (DSDMA),
2,2-dilinoleyl-4-(2-dimethylaminoethyl)-[1,3]-dioxolane (DLin-KC2-DMA),
1,2-dioleyloxy-N,N-dimethylaminopropane (DODMA),
2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA),
(2R)-2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA (2R)),
(2S)-2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA (2S)),
1,2-dilinolenyloxy-N,N-dimethyl-3-aminopropane (DLenDMA),
2,5-bis((9z,12z)-octadeca-9,12,dien-1-yloxyl)benzyl-4-(dimethylamino)butnoate (LKY750),
8-[(2-hydroxyethyl)[6-oxo-6-(undecyloxy)hexyl]amino]-octanoic acid, 1-octylnonyl ester (SM-102),
2-hexyl-decanoic acid, 1,1′-[[(4-hydroxybutyl)imino]di-6,1-hexanediyl]ester (ALC-0315),
4-(dimethylamino)-butanoic acid, (10Z,13Z)-1-(9Z,12Z)-9,12-octadecadien-1-yl-10,13-nonadecadien-1-yl ester (DLin-MC3-DMA or MC3)
((4-hydroxybutyl)azanediyl)bis(hexane-6,1-diyl)bis(2-hexyldecanoate)), and
8-[(2-hydroxyethyl)[6-oxo-6-(undecyloxy)hexyl]amino]-octanoic acid, 1-octylnonyl ester.

10. The method of paragraph 8 or paragraph 9, wherein the neutral lipid is selected from the group consisting of 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), and sphingomyelin.

11. The method of any one of paragraphs 8 to 10, wherein the PEGylated lipid is selected from the group consisting of PEG-modified phosphatidylethanolamines, PEG-modified phosphatidic acids, PEG-modified ceramides, PEG-modified dialkylamines, PEG-modified diacylglycerols, and PEG-modified dialkylglycerols, optionally PEG-c-DOMG, PEG-DMG, PEG-DLPE, PEG-DMPE, PEG-DPPC, and PEG-DSPE.

12. The method of any one of paragraphs 8 to 11, wherein the structural lipid is selected from the group consisting of cholesterol, fecosterol, sitosterol, campesterol, stigmasterol, brassicasterol, ergosterol, tomatidine, tomatine, ursolic acid and alpha-tocopherol.

13. The method of paragraph 12, wherein the structural lipid is cholesterol and/or campesterol.

14. The method of any one of paragraphs 1 to 13, wherein the LNP comprises a lipid component comprising: about 25 mol % to about 60 mol % of an ionisable lipid; about 2 mol % to about 25 mol % neutral lipid; about 18.5 mol % to about 60 mol % structural lipid; and about 0.2 mol % to about 10 mol % of PEGylated lipid.

15. The method of any one of paragraphs 1 to 14, wherein the mRNA is a self-amplifying mRNA (sa-mRNA) or a conventional mRNA.

16. The method of any one of paragraphs 1 to 15, wherein the mRNA is greater than 500 nt in length.

17. The method of any one of paragraphs 1 to 15, wherein the RNA is between 10,000 nt and 15,000 nt in length.

18. The method of any one of paragraphs 1 to 17, wherein the anion exchanger is anion exchange resin or an anion exchange membrane.

19. The method of paragraph 18 wherein the anion exchange membrane is a Mustang® Q, Sartobind® Q, Chromasorb®, Capto® Q, Q Sepharose Fast Flow (QSFF), Poros® Q, Fractogel® EMD, Natrix® Q or Eshmuno® Q membrane.

20. The method of paragraph 19, wherein the anion exchange membrane is a Mustang® Q membrane.

21. The method of any one of paragraphs 1 to 20, further comprising eluting the unencapsulated RNA from the anion exchanger.

22. The method of any one of paragraphs 1 to 21, wherein the lipid nanoparticle has a diameter of from about 30 nm to about 160 nm.

23. A composition comprising an enriched an enriched population of lipid nanoparticles produced by the method of any one of paragraphs to 21.

24. The composition of paragraph 23, wherein at least 90% of the RNA is encapsulated within the LNP.

25. An enriched lipid nanoparticle composition comprising
(i) a plurality of lipid nanoparticles wherein each LNP comprises
ionizable lipid,
a neutral lipid,
a PEGylated lipid, and
a structural lipid; and
(ii) RNA
wherein at least 90% of the RNA is encapsulated within the LNP.

26. A pharmaceutical composition comprising an enriched an enriched population of lipid nanoparticles produced by the method of any one of paragraphs 1 to 22, and a pharmaceutically acceptable carrier.

27. A pharmaceutical composition comprising the enriched population of lipid nanoparticles of paragraph 25, and a pharmaceutically acceptable carrier.

28. A method of delivering a mRNA to a mammalian cell, including administering the pharmaceutical composition of paragraph 26 or 27, to a subject to thereby contact the cell with the lipid nanoparticle and deliver the mRNA to the cell.

29. The method of paragraph 28 wherein the cell is a cell of a human subject.

30. A method of producing a polypeptide of interest in a mammalian cell, including the step of contacting the cell with the pharmaceutical composition of paragraph 26 or 27.

31. A method of treating a disease, disorder or condition in a subject in need of such treatment, comprising administering the pharmaceutical composition of paragraph 26 or 27, to the subject to thereby treat the disease, disorder or condition.

32. Use of the pharmaceutical composition of paragraph 26 or 27, in the manufacture of a medicament for the treatment of a disease, disorder or condition.

33. The method of paragraph 31 or the use of paragraph 32 wherein the disease, disorder or condition is selected from the group consisting of a rare disease, an infectious disease, cancer, a proliferative disease, a genetic disease, an autoimmune disease, diabetes, a neurodegenerative disease, a cardiovascular disease, a reno-vascular disease and a metabolic disease.

34. A vaccine comprising the composition of paragraph 23 or 24, the enriched lipid nanoparticle composition of paragraph 25, or the pharmaceutical composition of paragraph 26 or 27.

35. The vaccine of paragraph 34, wherein the vaccine is selected from a tumor vaccine, an influenza vaccine, and a SARS, including a SARS-CoV-2, vaccine.

The present disclosure includes the following non-limiting Examples.

### EXAMPLES

### Example 1—Preparation of an Enriched Population of Lipid Nanoparticles

sa-RNA was prepared using in vitro transcription from a linearized plasmid template using standard methods. The sa-RNA encoded a H5 antigen from A/turkey/Turkey/1/2005. The following construct was prepared: F500.3 (SEQ ID NO: 1). Briefly, the DNA template encoding the self-replicating RNA was produced in competent Escherichia coli cells that were transformed with a DNA plasmid. Individual bacterial colonies were isolated and the resultant plasmid DNA amplified in E. coli cultures. Following fermentation, the plasmid DNA was isolated using Maxiprep DNA kit and linearized by restriction digest. Restriction enzymes were then removed using phenol/chloroform extraction and ethanol precipitation.

mRNA was made by in vitro transcription from the linearized DNA template using a T7 RNA polymerase. Subsequently, the DNA template was removed by DNase digestion. Enzymatic capping using VCE was performed to add Cap0 and provide functional mRNA. The resultant mRNA was purified and resuspended in nuclease-free water.

An RNA-containing lipid nanoparticle composition was prepared using an ionizable cationic lipid, additional helper lipids and the sa-RNA produced as described above.

LKY750, 1,2-distearoyl-sn-glycero-3-phosphocholine (DSPC), cholesterol, and 1,2-dimyristoyl-rac-glycero-3-methylpolyoxyethylene (DMG-PEG2 k) were combined in a 40:10:48:2 molar ratio in ethanol at a concentration of 3.2 mM. A solution of mRNA in 50 mM citrate buffer at pH 6 was prepared at 0.025 mg/mL. The lipid solution in ethanol was then rapidly mixed with the mRNA in citrate buffer using a staggered herringbone micromixer such as a NanoAssemblr benchtop instrument (Precision Nanosystems). The total flow rate (TFR) was 12 mL/min and the flow rate ratio (FRR) was 2:1. This mixing ratio resulted in an 8:1 ratio of ionizable cationic lipids to RNA phosphate groups (N:P ratio) and a lipid to RNA mass ratio of 37:1. The mixed solution was diluted 10-fold into 50 mM citrate buffer at pH 6 and subjected to tangential flow filtration (TFF) using a 300 k molecular weight cut-off membrane (mPES) until concentrated to the original volume.

Subsequently, the citrate buffer was replaced with a buffer containing 20 mM Tris buffer at pH 7.5, 80 mM sodium chloride, and 3% sucrose using diafiltration with a 10-fold volume of the new buffer. The LNP solution was concentrated to a volume of between 5-10 mL, filtered using a 0.2 micron PES syringe filter, aliquoted into vials, and frozen at 1° C./min using a Corning® CoolCell® LX Cell Freezing Container until the samples reach −80° C. Samples will be stored at −80° C. until needed for further assays. Samples may be filtered with an anion exchanger before or after storage at −80° C.

After thawing, the sample was filtered with an anion exchange filter (Mustang Q, Pall Corporation) and the effluent collected.

LNPs containing sa-RNA were analysed to determine RNA concentration before anion exchange filtration and after anion exchange filtration (Table 1). The total amount of RNA contained in the sample and the percentage of that RNA that was encapsulated was determined using a fluorescence assay employing a dye that becomes more emissive upon binding RNA, such as Ribogreen. The total amount of RNA is determined by disrupting the LNP with a detergent to expose the encapsulated RNA, adding the dye, and comparing the emission intensity against a standard curve prepared using ribosomal RNA. It was though that the amount of unencapsulated RNA could be estimated in a similar manner by omitting detergent disruption of the LNP. With the total amount of RNA known and the amount of unencapsulated RNA known, the percent encapsulated RNA can be calculated thus:

$$Percent Encapsulation ( \% ) = ( ( {RNA}_{TOTAL} - {RNA}_{UNENCAPSULATED} ) / {RNA}_{TOTAL} ) \times 100$$

where RNA_{TOTAL} and RNA_{UNENCAPSULATED} are, respectively, the concentrations of total RNA and unencapsulated RNA.

TABLE 1

| | Encapsulation | |
|---|---|---|
| | (%) from | Concentration |
| LNP | Ribogreen assay | (ug/mL) |
| Unfiltered LNP | 99.1 | 62.5 |
| Anion exchange | 99.7 | 7.0 |
| filtered LNP | | |
| Flow-through | 98.8 | 5.5 |
| Un-filtered | 92.2 | 73.9 |

It was observed that Mustang filtration reduced the total amount of RNA present in the composition by 88.8% suggesting that only 11.2% of the RNA is completely encapsulated in the LNP prior to treatment with an anion exchanger.

### Example 2: Encapsulation Percentage

In order to assess whether the reduction in total RNA was due to a loss of RNA incompletely encapsulated in the LNP, empty LNP were formulated as described in Example 1 by mixing the lipid mixture with 50 mM citrate buffer at pH 6. sa-RNA was then combined with the empty LNP at an 8:1 N:P ratio and a final concentration of 42 μg/mL. The LNP were not frozen. The formulations containing empty LNP and empty LNP+RNA were filtered through an anion exchange filter and the effluent collected. The resulting formulations were then analysed to determine RNA concentration before anion exchange filtration and after anion exchange filtration (Table 2).

TABLE 2

| | Encapsulation | |
|---|---|---|
| | (%) from | Concentration |
| LNP | Ribogreen assay | (ug/mL) |
| Empty LNP | 101.4 | 3.5 |
| Empty LNP + | 90.9 | 43.5 |
| RNA | | |
| Filtered (Empty | 100.3 | 4 |
| LNP + RNA) | | |

From Table 2, it is evident that the Ribogreen assay does not measure percentage of RNA encapsulation in LNPs, or accurately report RNA concentration. In addition, the anion exchanger appears to remove RNA complexed to the outside of LNPs.

### Example 3: In Vitro Activity and Potency of Enriched LNP

The ability of the RNA-LNPs before and after treatment with an anion exchanger to transfect cultured cells was characterized using an in vitro assay quantifying the percentage of cells expressing the antigen of interest. RNA-LNPs were prepared as described in Example 1. However, the RNA included in the RNA-LNP was a sa-mRNA encoding a HA and NA subtype from A/turkey/Turkey/1/2005. The following sa-mRNA construct described in WO2022/118226 was prepared: NSP1-4.SGP.H5.SGPv2.N1 (also referred to as F602; SEQ ID NO: 2).

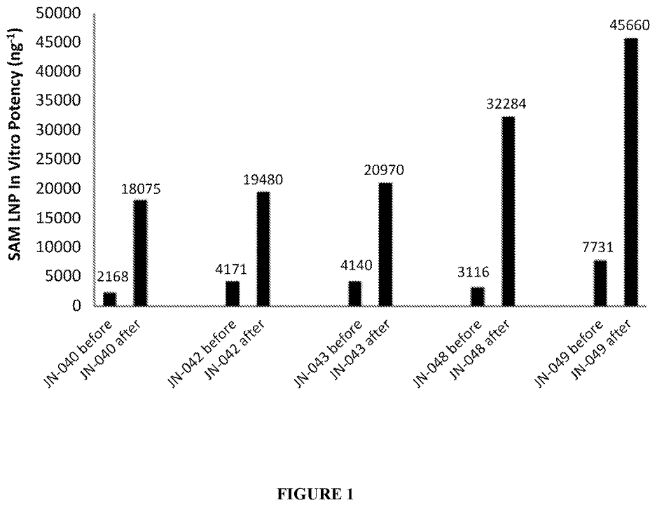

Two-fold serial dilutions of LNP-formulated sa-RNA prepared as described in Example 2 were either electroporated or transfected into a Baby Hamster Kidney (BHK) cell line. The SAM-encoded antigen was A/turkey/Turkey/05 (H5-sgpv2-N1). After 17-19 hrs, cells were harvested and stained for either S or N antigen expression using anti-S or anti-N antibodies. The number of cells positive for antigen expression and the mean fluorescence intensities (MFIs) were measured by AF4-MALS and fluorescence-activated cell sorting (FACS). Data were analysed to calculate the specific potency values (the probability of successful transfection per unit of mass of RNA) and the in vitro activity and potency of filtered (“after”) and unfiltered (“before”) LNPs is shown in FIG. 1. Potency values were based on H5+N1+co-expression. For each LNP formulation tested, the potency was between 4.6 and 10 fold higher after MustangQ filtration.

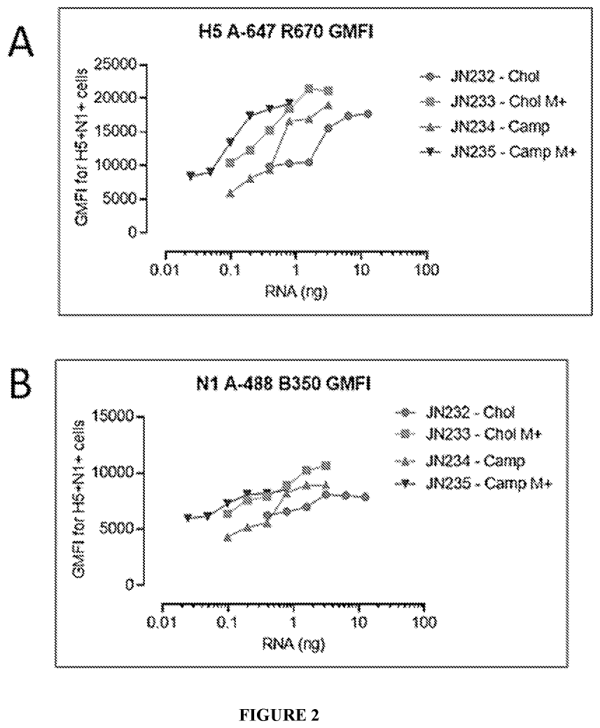

Another series of test LNPs were prepared to measure in vitro activity and potency. These included SAM-H5-N1/LNP.cholesterol, SAM-H5-N1/LNP.campesterol, SAM-H5-N1/LNP.cholesterol MustangQ-filtered, and SAM-H5-N1/LNP.campesterol MustangQ-filtered. The SAM-encoded antigen was A/turkey/Turkey/05 (H5-sgpv2-N1). The in vitro activity and potency of these LNP encapsulated vaccines was determined by measuring co-expression of H5 and N1. Results are shown in Table 3 and FIG. 2. Potency values were based on H5+N1+ co-expression.

TABLE 3
In vitro activity and potency for enriched and unenriched LNPs

| | | SAM | RNA | LNP |
|---|---|---|---|---|
| | Encapsulation | Conc. | activity | potency |
| LNP | (%) | (μg/mL) | (ng⁻¹) | (ng⁻¹) |
| SAM-H5- | 97.5 | 40.7 | 115 | 2788 |
| N1/LNP.cholesterol | | | | |
| SAM-H5- | 99.8 | 5.4 | 115 | 25280 |
| N1/LNP.cholesterol, | | | | |
| Mustang-filtered | | | | |
| SAM-H5- | 88.8 | 32.8 | 115 | 22020 |
| N1/LNP.campesterol | | | | |
| SAM-H5- | 98.2 | 3.9 | 115 | 97930 |
| N1/LNP.campesterol, | | | | |
| Mustang filtered | | | | |

Geometric mean fluorescence intensity (GMFI) values suggest higher levels of protein expression per cell for Mustang-filtered LNPs when compared to unfiltered LNPs. It was also observed that campesterol-containing LNPs show slightly higher levels of protein expression than cholesterol-containing LNPs.

### Example 4: Enriched LNP Induces Cell-Mediated Immune Responses

The ability of a composition of enriched LNPs to act as a vaccine was evaluated by measuring the antibody- and cell-based immune response following a prime-boost vaccination schedule. A priming vaccination was given on Day 0 via intramuscular injection (i.m.) and was followed 21 days later with a boosting vaccination. BALB/c female mice were vaccinated with either SAM-H5-N1/LNP.cholesterol, SAM-H5-N1/LNP.campesterol, SAM-H5-N1/LNP.cholesterol, Mustang-filtered, SAM-H5-N1/LNP.campesterol, Mustang-filtered, or H5N1 subunit+MF59. The SAM-encoded antigen was A/turkey/Turkey/05 (H5-sgpv2-N1). 10 mice were included in each group. Mice were given two doses of vaccine containing either 1, 0.1, 0.01 or 0.001 μg RNA. The H5N1 subunit+MF59 vaccine contained 1 μg H5 protein (SRID). Mice were bled the day before the first vaccination, 21 days post first vaccination (day 20), and another 21 days after the second vaccination (day 42).

To assess antibody responses, serum was collected at the end of study (i.e., 42 days after first or 21 days after the last, second vaccine dose). Serology was also assessed for the filtered and unfiltered LNP in a hemagglutination inhibition (HAI) assay, pseudovirus microneutralisation assay and anti-NA inhibition assay. All constructs induced virus specific antibodies.

For all serological assays, sera were treated in the same way, with Vibrio cholerae neuraminidase, also known as receptor-destroying enzyme (RDE) (Denka Seiken Co. Ltd., Tokyo, Japan) and diluted to a starting dilution of 1:10 with PBS. Sheep serum to H5N1 virus (FDA/CBER Kensington lot nu. H5-Ag-1115) was used as positive control sera.

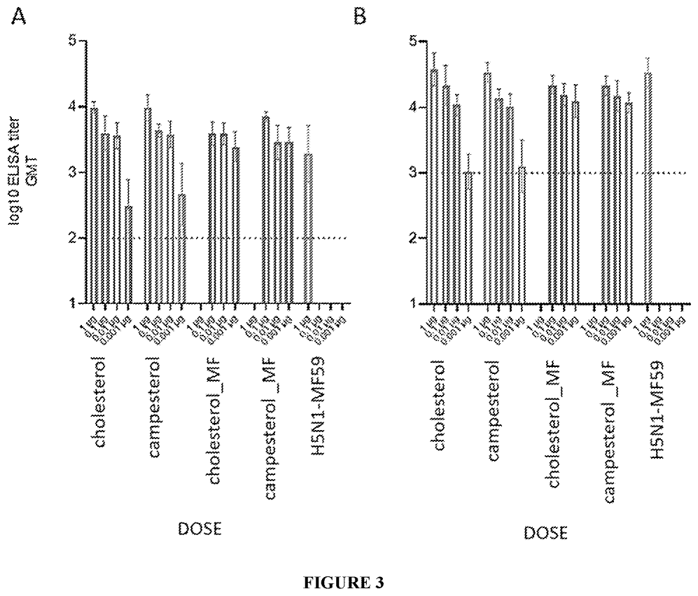

Total IgG levels were assessed by ELISA (FIG. 3 and Table 4).

TABLE 4
Total IgG levels as assessed by ELISA on day 21 and day 42

| | Cholesterol | | Campesterol | | Cholesterol_MF | | Campesterol_MF | | H5N1-MF 59 | |
|---|---|---|---|---|---|---|---|---|---|---|
| Day | 21 | 42 | 21 | 42 | 21 | 42 | 21 | 42 | 21 | 42 |
| 1 μg | 9,764 | 37,723 | 9,627 | 34,472 | | | | | 1,929 | 34,148 |
| 0.1 μg | 3,947 | 21,641 | 4,345 | 13,598 | 3,928, | 22,085 | 7,125 | 21,441 | | |
| 0.01 μg | 3,624 | 10,896 | 3,767 | 10,375 | 3,869 | 15,565 | 2,873 | 14,804 | | |
| 0.001 μg | 307 | 1,039 | 465 | 1,258 | 2,448 | 12,551 | 2,920 | 11,680 | | |

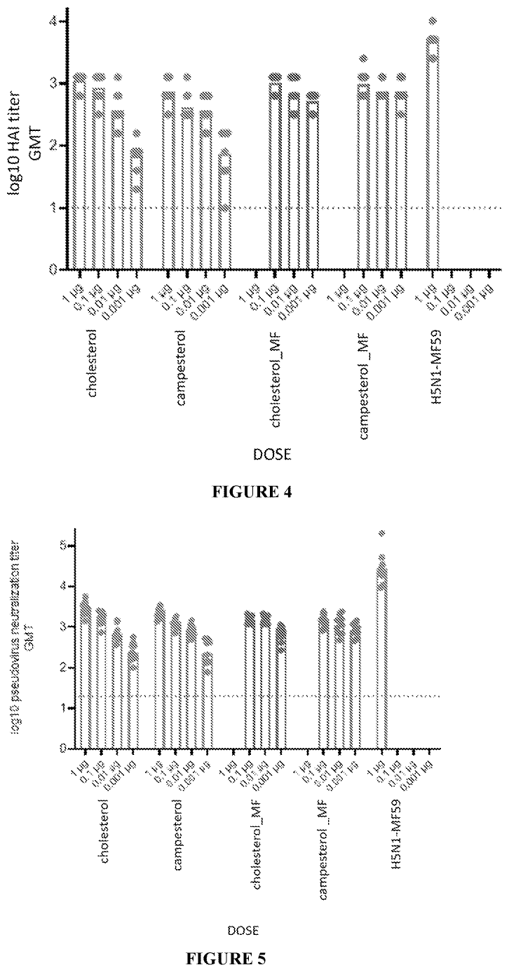

The results of the HAI assay are shown in FIG. 4 and Table 5.

TABLE 5
HAI titres on day 42

| | | | Cholesterol_ | Campesterol_ | H5N1- |
|---|---|---|---|---|---|
| | Cholesterol | Campesterol | MF | MF | MF 59 |
| 1 μg | 1,114 | 735 | | | 4,777 |
| 0.1 μg | 844 | 422 | 1,040 | 970 | |
| 0.01 μg | 368 | 368 | 686 | 735 | |
| 0.001 μg | 70 | 75 | 520 | 735 | |

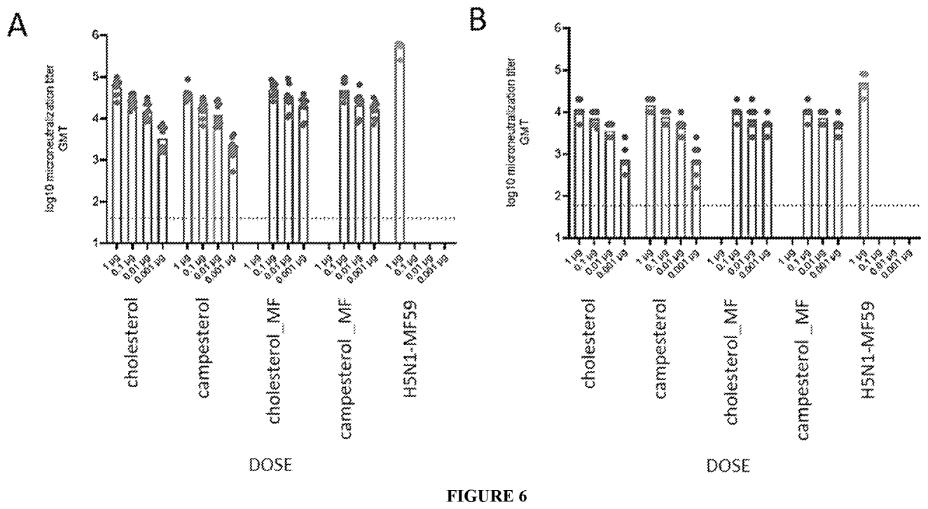

The results of the pseudovirus microneutralisation assay are shown in FIG. 5 and Table 6.

TABLE 6
Pseudovirus microneutralisation titres on day 42

| | | | Cholesterol_ | Campesterol_ | H5N1- |
|---|---|---|---|---|---|
| | Cholesterol | Campesterol | MF | MF | MF 59 |
| 1 μg | 2,801 | 2,121 | | | 26,913 |
| 0.1 μg | 1,679 | 1,174 | 1,521 | 1,495 | |
| 0.01 μg | 687 | 808 | 1,521 | 1,138 | |
| 0.001 μg | 239 | 221 | 705 | 839 | |

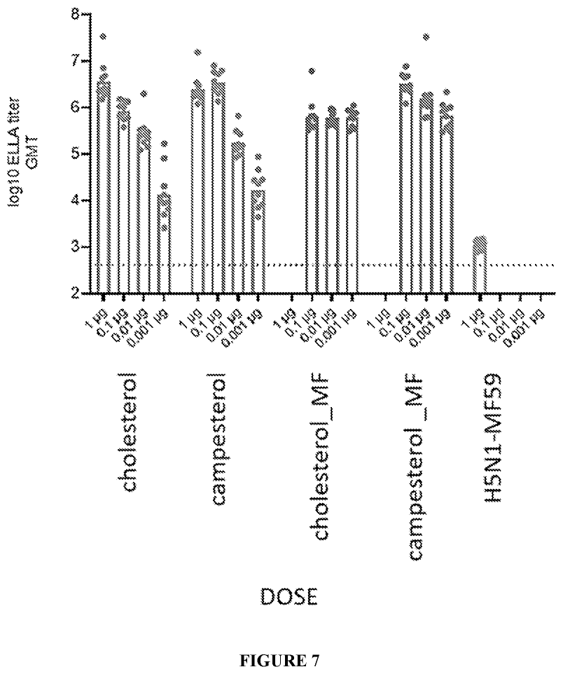

The results of the short form and long form microneutralisation assay are shown in FIGS. 6A and 6B and Table 7 and 8, respectively.

TABLE 7
short form microneutralisation titres on day 42

| | Cho- | | Cholesterol_ | Campesterol_ | H5N1- |
|---|---|---|---|---|---|
| | lesterol | Campesterol | MF | MF | MF 59 |
| 1 μg | 55,819 | 37,178 | | | 544,078 |
| 0.1 μg | 26,208 | 18,133 | 50,501 | 47,684 | |
| 0.01 μg | 14,907 | 12,347 | 30,304 | 21,015 | |
| 0.001 μg | 3,340 | 1,863 | 19,944 | 16,253 | |

TABLE 8
long form microneutralisation titres on day 42

| | | | Cholesterol_ | Campesterol_ | H5N1- |
|---|---|---|---|---|---|
| | Cholesterol | Campesterol | MF | MF | MF 59 |
| 1 μg | 11,763 | 14,482 | | | 50,428 |
| 0.1 μg | 7,085 | 7,760 | 10,240 | 10,240 | |
| 0.01 μg | 3,620 | 4,457 | 6,756 | 7,241 | |
| 0.001 μg | 735 | 735 | 4,777 | 4,457 | |

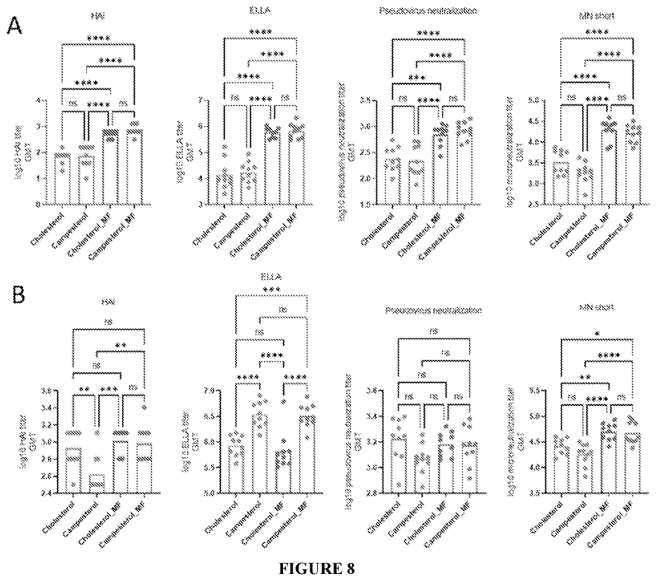

The results of the ELLA assay (for measuring NA inhibition) is shown in FIG. 7 and Table 9.

TABLE 9
ELLA titres on day 42

| | | | Cholesterol_ | Campesterol_ | H5N1- |
|---|---|---|---|---|---|
| | Cholesterol | Campesterol | MF | MF | MF 59 |
| 1 μg | 3,696,924 | 2,457,189 | | | 1147 |
| 0.1 μg | 856,643 | 3,470,853 | 661,099 | 3,234,482 | |
| 0.01 μg | 281,429 | 176,780 | 596,188 | 1,613,570 | |
| 0.001 μg | 13,863 | 17,438 | 614,169 | 673,287 | |

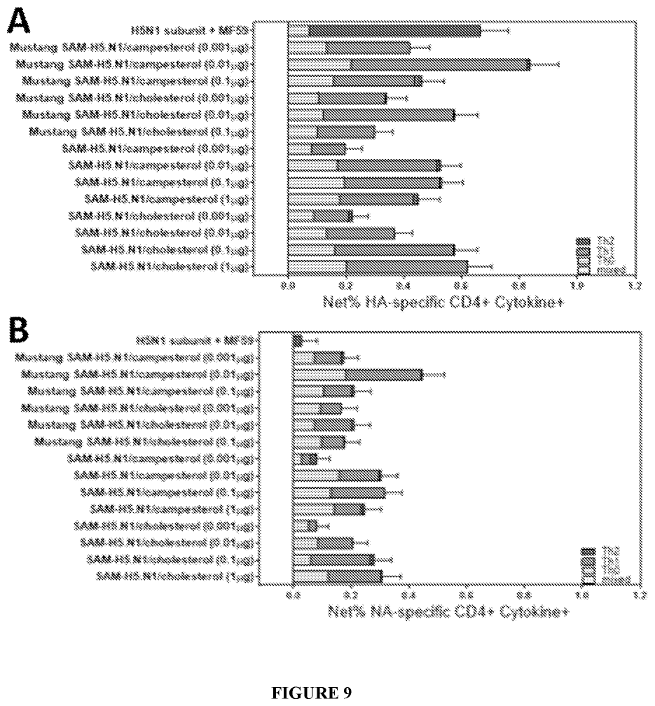

No difference was observed between LNP containing cholesterol and campesterol. Overall (FIG. 8), filtered LNP performed differently to the unfiltered LNP in all assays except ELLA (FIGS. 3-7). At lower doses (e.g. 0.001 μg), the filtered LNP performed consistently better that the unfiltered LNP (FIG. 8A). At higher doses (e.g. 0.1 μg), the differences between the filtered and unfiltered LNP were inconsistent (FIG. 8B).

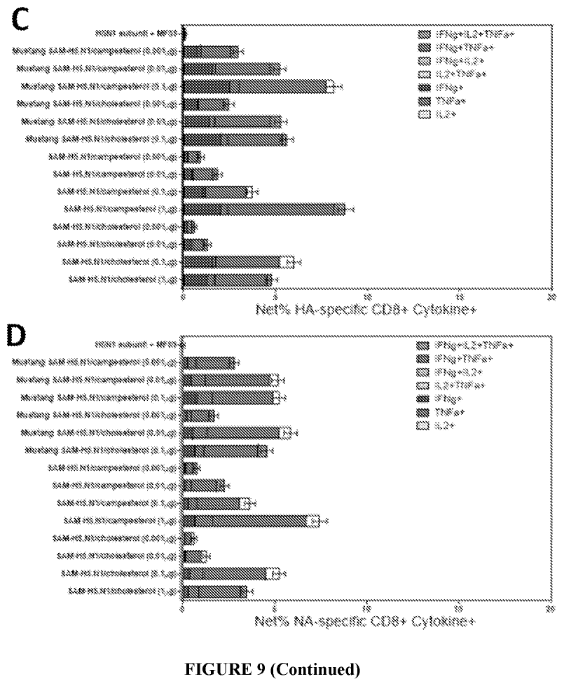

Spleens were collected, pooled and assayed for antigen-specific CD4 and CD8 T cells, using an in vitro antigen stimulation/intracellular cytokines immunofluorescence flow cytometry assay. The % CD4 or CD8 T cells that produced cytokines (one or more of IL-2, IFN-g, TNF-α, IL-5, IL-13 according to Table 10) were quantified. Overall, filtration did not impact the generation of cell-mediated immune response. Antigen specific CD4 and CD8 T cell responses are shown in FIG. 9.

TABLE 10
Cytokines secreted by specific T cells.

| Cytokine | Th0 | Th1 | Th2 | Th mixed |
|---|---|---|---|---|
| IFNγ | − | + | − | + |
| IL5 and/or IL13 | − | − | + | + |
| Il2 and/or TNFα | + | +/− | +/− | +/− |

CD 4 T cell responses were generated with both formulations. CD4 T cells elicited by the LNP vaccine were mostly Th0 (IL2+ and/or TNFa+, IFNg−, IL5−, IL13−) and Th1 (IFNg+, IL5−, IL13−) with few or no few or no mixed responses (FIG. 9A). Filtration using an anion exchanger did not alter Th type. As shown in FIG. 9B, immunization with the both filtered and unfiltered LNPs induces similar CD8 T cell responses.

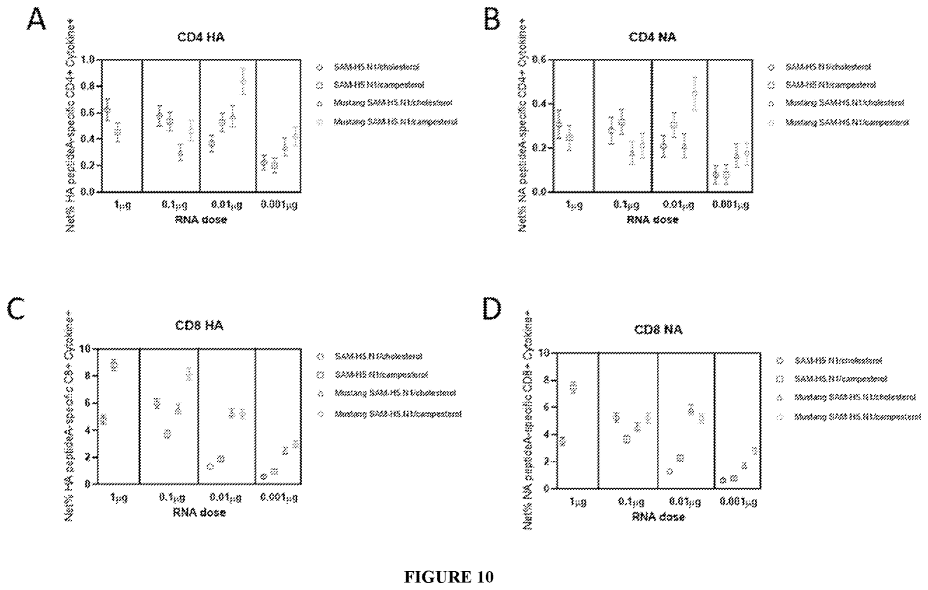

As shown in FIG. 10, the filtered LNP performed better at lower doses. However, the improvements resulting from filtration may plateau at a dose of 0.1 μg. It was also observed that in most cases the response from LNP containing cholesterol was better than the response from LNP containing campesterol.

Overall, LNP filtered using an anion exchanger generated better Ab and T cell responses at lower doses.

### Example 5: Microneutralization Assays

Microneutralization assays, short and long form, were performed in a qualified mammalian cell line (proprietary 33016-PF Madin-Darby Canine Kidney (MDCK)).

### Microneutralization Assay Short Form (MN Assay SF)

Virus fluorescent focus-based microneutralization (FFA MN) assay was performed using in house developed protocol. RDE treated test mouse samples and positive control sera were heat inactivated, diluted to a starting dilution of 1:40 with PBS, and fourfold serial diluted using the U-Bottom 96 well plate (BD Falcon) in neutralization medium (comprised of minimum essential medium D-MEM (GIBCO), supplemented with 1% BSA (Rockland, BSA-30), 100 U/mL penicillin and 100 μg/mL streptomycin (GIBCO)). A/turkey/Turkey/1/2005 (H5N1) virus was diluted to ~1,000-1,500 fluorescent focus-forming units (FFU)/well (20,000-30,000 FFU/mL) in neutralization medium and added in a 1:1 ratio to diluted serum.

After incubation for 2 h at 37° C., 5% CO₂, plates (Half Area 96 well plate, Corning) containing MDCK 33016-PF cells were inoculated with this mixture and incubated overnight for 16-18 h at 37° C. with 5% CO₂. MDCK 33016-PF cells had been seeded as 3.0E4/well (3.0E6/plate) at 6-8 h earlier in the cell growth medium (comprised of D-MEM, supplemented with 10% HyClone fetal bovine serum—FBS (Gibco), 100 U/mL penicillin and 100 ug/mL streptomycin). Following the overnight incubation and prior to immunostaining, cells were fixed with cold mixture of acetone and methanol.

The virus was visualized using separate 1 h incubations at room temperature of monoclonal antibodies specific to the nucleoproteins (NP) of the influenza A viruses (clones A1, A3 Blend, Millipore cat. no. MAB8251) and Alexa Fluor 488 Goat Anti-Mouse IgG (H+L) Ab (Invitrogen cat. no. A11001) diluted in PBS buffer containing 0.05% tween-20 (Sigma) and 2% BSA (Fraction V, Calbiochem, 2960, 1194C175). NP viral protein was quantified by a CTL Immunospot analyzer (Cellular Technology Limited, Shaker Heights, Cleveland, OH), using a fluorescein isothiocyanate (FITC) fluorescence filter set with excitation and emission wavelengths of 482 and 536 nm. Fluorescent foci were enumerated by use of software Immunospot 7.0.12.1 professional analyzer DC, using a custom analysis module. The data were successively logged by this software into an Excel data analysis spreadsheet, then 60% focus reduction endpoint was calculated from the average foci count of virus control wells (for each plate), and 60% focus reduction neutralization titer was calculated by linear interpolation between wells immediately above and below the 60% endpoint (for each sample).

### Microneutralization Assay Long Form (MN Assay LF)

MN assay LF was performed using in house developed protocol. RDE treated test mouse samples and positive control sera were heat inactivated, diluted to a starting dilution of 1:40 with PBS, and twofold serial diluted using the U-Bottom 96 well plate (BD Falcon) in neutralization medium (comprised of the 30% spent growth media (Irvine Scientific) and 70% infective media (protein free media—33016 MDCK PFM; GIBCO) supplemented with 100 U/mL penicillin, 100 μg/mL streptomycin (GIBCO), and 0.33 ug/mL TPCK-trypsin (TPCK treated, Tosyl phenylalanyl chloromethyl ketone, Sigma). A/turkey/Turkey/1/2005 (H5N1) virus was diluted to 100TCID (tissue culture infectious dose) per well in neutralization medium and added in a 1:1 ratio to diluted serum. Serially pre-diluted serum samples are incubated with the virus and allowed to react for 1 h at 37° C., 5% CO₂. In inoculation step, plates (Cell Culture 96-well plate, Costar) containing MDCK 33016-PF cells (which had been seeded as 3.0E4/well (3.0E6/plate) at day before in the antibiotic free cell growth medium (Irvine Scientific) were washed with sterile PBS, then infected with this mixture and incubated for 1 h at 37° C. with 5% CO₂. Infection was stopped by aspiration of antibody/virus mixture and washed cells with sterile PBS are inoculated with neutralizing media (100ul/well) containing twofold serially diluted antibodies, then incubated for 5 days at 37° C. with 5% CO₂. In the final “read-out” step, detection of virus was performed by HA quantification of the virus using 0.5% turkey red blood cells (Lampire Biological Laboratories). The absence of infectivity constitutes a positive neutralization reaction and indicates the presence of virus-specific antibodies in the serum sample.

### Example 6: Hemagglutination Inhibition (HAI) Assay

HAI assay was performed as previously described (WHO (2011) Manual for the laboratory diagnosis and virological surveillance of influenza: WHO Press, World Health Organization, 20 Avenue Appia, 1211 Geneva 27, Switzerland). Briefly, RDE treated test mouse samples and positive control sera were heat inactivated, diluted to a starting dilution of 1:10 with PBS, and twofold serial diluted samples (25 μl) were incubated with equal volumes of viruses (4 hemagglutinating units [HAU]) of A/turkey/Turkey/1/2005 (H5N1) at room temperature (RT) for 30 minutes. Then, an equal volume of 0.5% turkey red blood cells (Lampire Biological Laboratories) was added and incubated at RT for 30 minutes. The HAI titer was expressed as the reciprocal of the highest dilution of the samples inhibiting hemagglutination.

SEQUENCES

| SEQ | |
|---|---|
| ID | |
| NO: | Sequence (5′ to 3′) |
| 1 | ataggcggcgcatgagagaagcccagaccaattacctacccaaaatggagaaagttcacgttgacatcgaggaagacagccc |
| | attcctcagagctttgcagcggagcttcccgcagtttgaggtagaagccaagcaggtcactgataatgaccatgctaatgccaga |
| | gcgttttcgcatctggcttcaaaactgatcgaaacggaggtggacccatccgacacgatccttgacattggaagtgcgcccgccc |
| | gcagaatgtattctaagcacaagtatcattgtatctgtccgatgagatgtgcggaagatccggacagattgtataagtatgcaacta |
| | agctgaagaaaaactgtaaggaaataactgataaggaattggacaagaaaatgaaggagctcgccgccgtcatgagcgaccct |
| | gacctggaaactgagactatgtgcctccacgacgacgagtcgtgtcgctacgaagggcaagtcgctgtttaccaggatgtatac |
| | gcggttgacggaccgacaagtctctatcaccaagccaataagggagttagagtcgcctactggataggctttgacaccacccctt |
| | ttatgtttaagaacttggctggagcatatccatcatactctaccaactgggccgacgaaaccgtgttaacggctcgtaacataggcc |
| | tatgcagctctgacgttatggagcggtcacgtagagggatgtccattcttagaaagaagtatttgaaaccatccaacaatgttctatt |
| | ctctgttggctcgaccatctaccacgagaagagggacttactgaggagctggcacctgccgtctgtatttcacttacgtggcaagc |
| | aaaattacacatgtcggtgtgagactatagttagttgcgacgggtacgtcgttaaaagaatagctatcagtccaggcctgtatggg |
| | aagccttcaggctatgctgctacgatgcaccgcgagggattcttgtgctgcaaagtgacagacacattgaacggggagagggtc |
| | tcttttcccgtgtgcacgtatgtgccagctacattgtgtgaccaaatgactggcatactggcaacagatgtcagtgcggacgacgc |
| | gcaaaaactgctggttgggctcaaccagcgtatagtcgtcaacggtcgcacccagagaaacaccaataccatgaaaaattacctt |
| | ttgcccgtagtggcccaggcatttgctaggtgggcaaaggaatataaggaagatcaagaagatgaaaggccactaggactacg |
| | agatagacagttagtcatggggtgttgttgggcttttagaaggcacaagataacatctatttataagcgcccggatacccaaaccat |
| | catcaaagtgaacagcgatttccactcattcgtgctgcccaggataggcagtaacacattggagatcgggctgagaacaagaatc |
| | aggaaaatgttagaggagcacaaggagccgtcacctctcattaccgccgaggacgtacaagaagctaagtgcgcagccgatg |
| | aggctaaggaggtgcgtgaagccgaggagttgcgcgcagctctaccacctttggcagctgatgttgaggagcccactctggaa |
| | gccgatgtcgacttgatgttacaagaggctggggccggctcagtggagacacctcgtggcttgataaaggttaccagctacgat |
| | ggcgaggacaagatcggctcttacgctgtgctttctccgcaggctgtactcaagagtgaaaaattatcttgcatccaccctctcgct |
| | gaacaagtcatagtgataacacactctggccgaaaagggcgttatgccgtggaaccataccatggtaaagtagtggtgccagag |
| | ggacatgcaatacccgtccaggactttcaagctctgagtgaaagtgccaccattgtgtacaacgaacgtgagttcgtaaacaggt |
| | acctgcaccatattgccacacatggaggagcgctgaacactgatgaagaatattacaaaactgtcaagcccagcgagcacgac |
| | ggcgaatacctgtacgacatcgacaggaaacagtgcgtcaagaaagaactagtcactgggctagggctcacaggcgagctgg |
| | tggatcctcccttccatgaattcgcctacgagagtctgagaacacgaccagccgctccttaccaagtaccaaccataggggtgtat |
| | ggcgtgccaggatcaggcaagtctggcatcattaaaagcgcagtcaccaaaaaagatctagtggtgagcgccaagaaagaaa |
| | actgtgcagaaattataagggacgtcaagaaaatgaaagggctggacgtcaatgccagaactgtggactcagtgctcttgaatg |
| | gatgcaaacaccccgtagagaccctgtatattgacgaagcttttgcttgtcatgcaggtactctcagagcgctcatagccattataa |
| | gacctaaaaaggcagtgctctgcggggatcccaaacagtgcggtttttttaacatgatgtgcctgaaagtgcattttaaccacgag |
| | atttgcacacaagtcttccacaaaagcatctctcgccgttgcactaaatctgtgacttcggtcgtctcaaccttgttttacgacaaaa |
| | aaatgagaacgacgaatccgaaagagactaagattgtgattgacactaccggcagtaccaaacctaagcaggacgatctcattctc |
| | acttgtttcagaggggggtgaagcagttgcaaatagattacaaaggcaacgaaataatgacggcagctgcctctcaagggctg |
| | acccgtaaaggtgtgtatgccgttcggtacaaggtgaatgaaaatcctctgtacgcacccacctcagaacatgtgaacgtcctact |
| | gacccgcacggaggaccgcatcgtgtggaaaacactagccggcgacccatggataaaaacactgactgccaagtaccctggg |
| | aatttcactgccacgatagaggagtggcaagcagagcatgatgccatcatgaggcacatcttggagagaccggaccctaccga |
| | cgtcttccagaataaggcaaacgtgtgttgggccaaggctttagtgccggtgctgaagaccgctggcatagacatgaccactga |
| | acaatggaacactgtggattattttgaaacggacaaagctcactcagcagagatagtattgaaccaactatgcgtgaggttctttgg |
| | actcgatctggactccggtctattttctgcacccactgttccgttatccattaggaataatcactgggataactccccgtcgcctaac |
| | atgtacgggctgaataaagaagtggtccgtcagctctctcgcaggtacccacaactgcctcgggcagttgccactggaagagtcta |
| | tgacatgaacactggtacactgcgcaattatgatccgcgcataaacctagtacctgtaaacagaagactgcctcatgctttagtcct |
| | ccaccataatgaacacccacagagtgacttttcttcattcgtcagcaaattgaagggcagaactgtcctggtggtcggggaaaagt |
| | tgtccgtcccaggcaaaatggttgactggttgtcagaccggcctgaggctaccttcagagctcggctggatttaggcatcccagg |
| | tgatgtgcccaaatatgacataatatttgttaatgtgaggaccccatataaataccatcactatcagcagtgtgaagaccatgccatt |
| | aagcttagcatgttgaccaagaaagcttgtctgcatctgaatcccggcggaacctgtgtcagcataggttatggttacgctgacag |
| | ggccagcgaaagcatcattggtgctatagcgcggcagttcaagttttcccgggtatgcaaaccgaaatcctcacttgaagagacg |
| | gaagttctgtttgtattcattgggtacgatcgcaaggcccgtacgcacaatccttacaagctttcatcaaccttgaccaacatttata |
| | caggttccagactccacgaagccggatgtgcaccctcatatcatgtggtgcgaggggatattgccacggccaccgaaggagtgat |
| | tataaatgctgctaacagcaaaggacaacctggcggaggggtgtgcggagcgctgtataagaaattcccggaaagcttcgattt |
| | acagccgatcgaagtaggaaaagcgcgactggtcaaaggtgcagctaaacatatcattcatgccgtaggaccaaacttcaacaa |
| | agtttcggaggttgaaggtgacaaacagttggcagaggcttatgagtccatcgctaagattgtcaacgataacaattacaagtcag |
| | tagcgattccactgttgtccaccggcatcttttccgggaacaaagatcgactaacccaatcattgaaccatttgctgacagctttaga |
| | caccactgatgcagatgtagccatatactgcagggacaagaaatgggaaatgactctcaaggaagcagtggctaggagagaag |
| | cagtggaggagatatgcatatccgacgactcttcagtgacagaacctgatgcagagctggtgagggtgcatccgaagagttcttt |
| | ggctggaaggaagggctacagcacaagcgatggcaaaactttctcatatttggaagggaccaagtttcaccaggcggccaagg |
| | atatagcagaaattaatgccatgtggcccgttgcaacggaggccaatgagcaggtatgcatgtatatcctcggagaaagcatgag |
| | cagtattaggtcgaaatgccccgtcgaagagtcggaagcctccacaccacctagcacgctgccttgcttgtgcatccatgccatg |
| | actccagaaagagtacagcgcctaaaagcctcacgtccagaacaaattactgtgtgctcatcctttccattgccgaagtatagaatc |
| | actggtgtgcagaagatccaatgctcccagcctatattgttctcaccgaaagtgcctgcgtatattcatccaaggaagtatctcgtg |
| | gaaacaccaccggtagacgagactccggagccatcggcagagaaccaatccacagaggggacacctgaacaaccaccactt |
| | ataaccgaggatgagaccaggactagaacgcctgagccgatcatcatcgaagaggaagaagaggatagcataagtttgctgtc |
| | agatggcccgacccaccaggtgctgcaagtcgaggcagacattcacgggccgccctctgtatctagctcatcctggtccattcct |
| | catgcatccgactttgatgtggacagtttatccatacttgacaccctggagggagctagcgtgaccagcggggcaacgtcagcc |
| | gagactaactcttacttcgcaaagagtatggagtttctggcgcgaccggtgcctgcgcctcgaacagtattcaggaaccctccac |
| | atcccgctccgcgcacaagaacaccgtcacttgcacccagcagggcctgctcgagaaccagcctagtttccaccccgccaggc |
| | gtgaatagggtgatcactagagaggagctcgaggcgcttaccccgtcacgcactcctagcaggtcggtctcgagaaccagcct |
| | ggtctccaacccgccaggcgtaaatagggtgattacaagagaggagtttgaggcgttcgtagcacaacaacaatgacggtttga |
| | tgcgggtgcatacatcttttcctccgacaccggtcaagggcatttacaacaaaaatcagtaaggcaaacggtgctatccgaagtg |
| | gtgttggagaggaccgaattggagatttcgtatgccccgcgcctcgaccaagaaaaagaagaattactacgcaagaaattacag |
| | ttaaatcccacacctgctaacagaagcagataccagtccaggaaggtggagaacatgaaagccataacagctagacgtattctg |
| | caaggcctagggcattatttgaaggcagaaggaaaagtggagtgctaccgaaccctgcatcctgttcctttgtattcatctagtgtg |
| | aaccgtgccttttcaagccccaaggtcgcagtggaagcctgtaacgccatgttgaaagagaactttccgactgtggcttcttactgt |
| | attattccagagtacgatgcctatttggacatggttgacggagcttcatgctgcttagacactgccagtttttgccctgcaaagctgc |
| | gcagctttccaaagaaacactcctatttggaacccacaatacgatcggcagtgccttcagcgatccagaacacgctccagaacgt |
| | cctggcagctgccacaaaaagaaattgcaatgtcacgcaaatgagagaattgcccgtattggattcggcggcctttaatgtggaat |
| | gcttcaagaaatatgcgtgtaataatgaatattgggaaacgtttaaagaaaaccccatcaggcttactgaagaaaacgtggtaaatt |
| | acattaccaaattaaaaggaccaaaagctgctgctctttttgcgaagacacataatttgaatatgttgcaggacataccaatggaca |
| | ggtttgtaatggacttaaagagagacgtgaaagtgactccaggaacaaaacatactgaagaacggcccaaggtacaggtgatcc |
| | aggctgccgatccgctagcaacagcgtatctgtgcggaatccaccgagagctggttaggagattaaatgcggtcctgcttccga |
| | acattcatacactgtttgatatgtcggctgaagactttgacgctattatagccgagcacttccagcctggggattgtgttctggaaac |
| | tgacatcgcgtcgtttgataaaagtgaggacgacgccatggctctgaccgcgttaatgattctggaagacttaggtgtggacgcag |
| | agctgttgacgctgattgaggcggctttcggcgaaatttcatcaatacatttgcccactaaaactaaatttaaattcggagccatgat |
| | gaaatctggaatgttcctcacactgtttgtgaacacagtcattaacattgtaatcgcaagcagagtgttgagagaacggctaaccg |
| | gatcaccatgtgcagcattcattggagatgacaatatcgtgaaaggagtcaaatcggacaaattaatggcagacaggtgcgcca |
| | cctggttgaatatggaagtcaagattatagatgctgtggtgggcgagaaagcgccttatttctgtggagggtttattttgtgtgactc |
| | cgtgaccggcacagcgtgccgtgtggcagaccccctaaaaaggctgtttaagcttggcaaacctctggcagcagacgatgaaca |
| | tgatgatgacaggagaagggcattgcatgaagagtcaacacgctggaaccgagtgggtattctttcagagctgtgcaaggcagt |
| | agaatcaaggtatgaaaccgtaggaacttccatcatagttatggccatgactactctagctagcagtgttaaatcattcagctacctg |
| | agaggggcccctataactctctacggctaacctgaatggactacgacatagtctagtccgccaagatggagaaaatagtgcttctt |
| | cttgcaatagtcagccttgttaaaagtgatcagatttgcattggttaccatgcaaacaactcgacagagcaggttgacacaataatg |
| | gaaaagaacgtcactgttacacacgcccaagacatactggaaaagacacacaacgggaaactctgcgatctagatggagtgaa |
| | gcctctaattttaagagattgtagtgtagctggatggctcctcgggaacccaatgtgtgacgaattcctcaatgtgccggaatggtct |
| | tacatagtggagaagatcaatccagccaatgacctctgttacccagggaatttcaacgactatgaagaactgaaacacctattgag |
| | cagaataaaccattttgagaaaattcagatcatccccaaaagttcttggtcagatcatgaagcctcagcaggggtgagctcagcat |
| | gtccataccagggaaggtcctccttttttagaaatgtggtatggcttatcaaaaaggacaatgcatacccaacaataaagagaagtt |
| | acaataataccaaccaagaagatcttttggtattgtgggggattcaccatccaaatgatgcggcagagcagacaaggctctatcaa |
| | aacccaactacctatatttccgttgggacatcaacactaaaccagagattggtaccaaaaatagccactagatctaaggtaaacgg |
| | gcaaagtggaaggatggagttcttttggacaattttaaaaccgaatgatgcaataaactttgagagtaatggaaatttcattgctcca |
| | gaaaatgcatacaaaattgtcaagaaaggggactcaacaattatgaaaagtgagttggaatatggtaactgcaacaccaagtgtc |
| | aaactccaataggggcgataaactctagtatgccattccacaacatccaccctctcaccatcggggaatgccccaaatatgtgaaa |
| | tcaagcagattagtccttgctactgggctcagaaatagccctcaacgagagacacgaggactatttggagctatagcaggttttat |
| | agagggaggatggggaatggtagatggttggtatgggtaccaccatagcaacgagcaggggagtgggtacgctgcagacaa |
| | agaatccactcaaaaggcaatagatggagtcaccaataaggtcaactcgatcattgacaaaatgaacactcagtttgaggctgttg |
| | gaagggaatttaataacttagaaaggagaatagaaaatttaaacaagaagatggaagacggattcctagatgtctggacttataat |
| | gctgaacttctggttctcatggaaaatgagagaactctagactttcatgactcaaatgtcaagaacctttacgacaaggtccgacta |
| | cagcttagggataatgcaaaggagcttggtaacggttgtttcgagttctatcacagatgtgataatgaatgtatggaaagtgtaaga |
| | aacggaacgtatgactacccgcagtattcagaagaagcaagattaaaaagagaggaaataagtggagtaaaattggaatcaata |
| | ggaacttaccaaatactgtcaatttattcaacagtggcgagctccctagcactggcaatcatggtggctggtctatctttatggatgt |
| | gctccaatggatcgttacaatgcagaatttgcatttaaggcgcgcccacccagcggccgcccgctacgccccaatgatccgacc |
| | agcaaaactcgatgtacttccgaggaactgatgtgcataatgcatcaggctggtacattagatccccgcttaccgcgggcaatata |
| | gcaacactaaaaactcgatgtacttccgaggaagcgcagtgcataatgctgcgcagtgttgccacataaccactatattaaccattt |
| | atctagcggacgccaaaaactcaatgtatttctgaggaagcgtggtgcataatgccacgcagcgtctgcataacttttattatttcttt |
| | tattaatcaacaaaatttgtttttaacatttcaaaaaaaaaaaaaaaaaaaaaaaaaaaaaaaaaaaaagaagagcgtttaaacac |
| | gtgatatctggcctcatgggccttcctttcactgcccgctttccagtcgggaaacctgtcgtgccagctgcattaacatggtcatagc |
| | tgtttccttgcgtattgggcgctctccgcttcctcgctcactgactcgctgcgctcggtcgttcgggtaaagcctggggtgcctaatg |
| | agcaaaaggccagcaaaaggccaggaaccgtaaaaaggccgcgttgctggcgtttttccataggctccgcccccctgacgag |
| | catcacaaaaatcgacgctcaagtcagaggtggcgaaacccgacaggactataaagataccaggcgtttccccctggaagctc |
| | cctcgtgcgctctcctgttccgaccctgccgcttaccggatacctgtccgcctttctcccttcgggaagcgtggcgctttctcatagc |
| | tcacgctgtaggtatctcagttcggtgtaggtcgttcgctccaagctgggctgtgtgcacgaaccccccgttcagcccgaccgctg |
| | cgccttatccggtaactatcgtcttgagtccaacccggtaagacacgacttatcgccactggcagcagccactggtaacaggatta |
| | gcagagcgaggtatgtaggcggtgctacagagttcttgaagtggtggcctaactacggctacactagaagaacagtatttggtat |
| | ctgcgctctgctgaagccagttaccttcggaaaaagagttggtagctcttgatccggcaaacaaaccaccgctggtagcggtggt |
| | ttttttgtttgcaggcagcagattacgcgcagaaaaaaaggatctcaagaagatcctttgatcttttctacggggtctgacgctcagt |
| | ggaacgaaaactcacgttaagggattttggtcatgaatacacggtgcctgactgcgttagcaatttaactgtgataaactaccgcat |
| | taaagcttatcgatgataagctgtcaaacatgagaattcttagaaaaactcatcgagcatcaaatgaaactgcaatttattcatatca |
| | ggattatcaataccatatttttgaaaaagccgtttctgtaatgaaggagaaaactcaccgaggcagttccataggatggcaagatcct |
| | ggtatcggtctgcgattccgactcgtccaacatcaatacaacctattaatttcccctcgtcaaaaataaggttatcaagtgagaaatc |
| | accatgagtgacgactgaatccggtgagaatggcaaaagcttatgcatttctttccagacttgttcaacaggccagccattacgctc |
| | gtcatcaaaatcactcgcatcaaccaaaccgttattcattcgtgattgcgcctgagcgagacgaaatacgcgatcgctgttaaaag |
| | gacaattacaaacaggaatcgaatgcaaccggcgcaggaacactgccagcgcatcaacaatattttcacctgaatcaggatattc |
| | ttctaatacctggaatgctgttttcccggggatcgcagtggtgagtaaccatgcatcatcaggagtacggataaaatgcttgatggt |
| | cggaagaggcataaattccgtcagccagtttagtctgaccatctcatctgtaacatcattggcaacgctacctttgccatgtttcaga |
| | aacaactctggcgcatcgggcttcccatacaatcgatagattgtcgcacctgattgcccgacattatcgcgagcccatttataccca |
| | tataaatcagcatccatgttggaatttaatcgcggcctcgagcaagacgtttcccgttgaatatggctcataacaccccttgtattac |
| | tgtttatgtaagcagacagttttattgttcatgagcggatacatatttgaatgtatttagaaaaataaacaaataggggttccgcgca |
| | catttccccgaaaagtgccacctaaattgtaagcgttaatattttgttaaaattcgcgttaaatttttgttaaatcagctcatttttt |
| | aaccaataggccgaaatcggcaaaatcccttataaatcaaaagaatagaccgagatagggttgagtggccgctacagggcgctcccat |
| | tcgccattcaggctgcgcaactgttgggaagggcgtttcggtgcgggcctcttcgctattacgccagctggcgaaagggggatgtgc |
| | tgcaaggcgattaagttgggtaacgccagggttttcccagtcacacgcgtaatacgactcactatag |
| 2 | ataggcggcgcatgagagaagcccagaccaattacctacccaaaatggagaaagttcacgttgacatcgaggaagacagccc |
| | attcctcagagctttgcagcggagcttcccgcagtttgaggtagaagccaagcaggtcactgataatgaccatgctaatgccaga |
| | gcgttttcgcatctggcttcaaaactgatcgaaacggaggtggacccatccgacacgatccttgacattggaagtgcgcccgccc |
| | gcagaatgtattctaagcacaagtatcattgtatctgtccgatgagatgtgcggaagatccggacagattgtataagtatgcaacta |
| | agctgaagaaaaactgtaaggaaataactgataaggaattggacaagaaaatgaaggagctcgccgccgtcatgagcgaccct |
| | gacctggaaactgagactatgtgcctccacgacgacgagtcgtgtcgctacgaagggcaagtcgctgtttaccaggatgtatac |
| | gcggttgacggaccgacaagtctctatcaccaagccaataagggagttagagtcgcctactggataggctttgacaccacccctt |
| | ttatgtttaagaacttggctggagcatatccatcatactctaccaactgggccgacgaaaccgtgttaacggctcgtaacataggcc |
| | tatgcagctctgacgttatggagcggtcacgtagagggatgtccattcttagaaagaagtatttgaaaccatccaacaatgttctatt |
| | ctctgttggctcgaccatctaccacgagaagagggacttactgaggagctggcacctgccgtctgtatttcacttacgtggcaagc |
| | aaaattacacatgtcggtgtgagactatagttagttgcgacgggtacgtcgttaaaagaatagctatcagtccaggcctgtatggg |
| | aagccttcaggctatgctgctacgatgcaccgcgagggattcttgtgctgcaaagtgacagacacattgaacggggagagggtc |
| | tcttttcccgtgtgcacgtatgtgccagctacattgtgtgaccaaatgactggcatactggcaacagatgtcagtgcggacgacgc |
| | gcaaaaactgctggttgggctcaaccagcgtatagtcgtcaacggtcgcacccagagaaacaccaataccatgaaaaattacctt |
| | ttgcccgtagtggcccaggcatttgctaggtgggcaaaggaatataaggaagatcaagaagatgaaaggccactaggactacg |
| | agatagacagttagtcatggggtgttgttgggcttttagaaggcacaagataacatctatttataagcgcccggatacccaaaccat |
| | catcaaagtgaacagcgatttccactcattcgtgctgcccaggataggcagtaacacattggagatcgggctgagaacaagaatc |
| | aggaaaatgttagaggagcacaaggagccgtcacctctcattaccgccgaggacgtacaagaagctaagtgcgcagccgatg |
| | aggctaaggaggtgcgtgaagccgaggagttgcgcgcagctctaccacctttggcagctgatgttgaggagcccactctggaa |
| | gccgatgtcgacttgatgttacaagaggctggggccggctcagtggagacacctcgtggcttgataaaggttaccagctacgat |
| | ggcgaggacaagatcggctcttacgctgtgctttctccgcaggctgtactcaagagtgaaaaattatcttgcatccaccctctcgct |
| | gaacaagtcatagtgataacacactctggccgaaaagggcgttatgccgtggaaccataccatggtaaagtagtggtgccagag |
| | ggacatgcaatacccgtccaggactttcaagctctgagtgaaagtgccaccattgtgtacaacgaacgtgagttcgtaaacaggt |
| | acctgcaccatattgccacacatggaggagcgctgaacactgatgaagaatattacaaaactgtcaagcccagcgagcacgac |
| | ggcgaatacctgtacgacatcgacaggaaacagtgcgtcaagaaagaactagtcactgggctagggctcacaggcgagctgg |
| | tggatcctcccttccatgaattcgcctacgagagtctgagaacacgaccagccgctccttaccaagtaccaaccataggggtgtat |
| | ggcgtgccaggatcaggcaagtctggcatcattaaaagcgcagtcaccaaaaaagatctagtggtgagcgccaagaaagaaa |
| | actgtgcagaaattataagggacgtcaagaaaatgaaagggctggacgtcaatgccagaactgtggactcagtgctcttgaatg |
| | gatgcaaacaccccgtagagaccctgtatattgacgaagcttttgcttgtcatgcaggtactctcagagcgctcatagccattataa |
| | gacctaaaaaggcagtgctctgcggggatcccaaacagtgcggtttttttaacatgatgtgcctgaaagtgcattttaaccacgag |
| | atttgcacacaagtcttccacaaaagcatctctcgccgttgcactaaatctgtgacttcggtcgtctcaaccttgttttacgacaaaa |
| | aaatgagaacgacgaatccgaaagagactaagattgtgattgacactaccggcagtaccaaacctaagcaggacgatctcattctc |
| | acttgtttcagagggtgggtgaagcagttgcaaatagattacaaaggcaacgaaataatgacggcagctgcctctcaagggctg |
| | acccgtaaaggtgtgtatgccgttcggtacaaggtgaatgaaaatcctctgtacgcacccacctcagaacatgtgaacgtcctact |
| | gacccgcacggaggaccgcatcgtgtggaaaacactagccggcgacccatggataaaaacactgactgccaagtaccctggg |
| | aatttcactgccacgatagaggagtggcaagcagagcatgatgccatcatgaggcacatcttggagagaccggaccctaccga |
| | cgtcttccagaataaggcaaacgtgtgttgggccaaggctttagtgccggtgctgaagaccgctggcatagacatgaccactga |
| | acaatggaacactgtggattattttgaaacggacaaagctcactcagcagagatagtattgaaccaactatgcgtgaggttctttgg |
| | actcgatctggactccggtctattttctgcacccactgttccgttatccattaggaataatcactgggataactccccgtcgcctaac |
| | atgtacgggctgaataaagaagtggtccgtcagctctctcgcaggtacccacaactgcctcgggcagttgccactggaagagtcta |
| | tgacatgaacactggtacactgcgcaattatgatccgcgcataaacctagtacctgtaaacagaagactgcctcatgctttagtcct |
| | ccaccataatgaacacccacagagtgacttttcttcattcgtcagcaaattgaagggcagaactgtcctggtggtcggggaaaagt |
| | tgtccgtcccaggcaaaatggttgactggttgtcagaccggcctgaggctaccttcagagctcggctggatttaggcatcccagg |
| | tgatgtgcccaaatatgacataatatttgttaatgtgaggaccccatataaataccatcactatcagcagtgtgaagaccatgccatt |
| | aagcttagcatgttgaccaagaaagcttgtctgcatctgaatcccggcggaacctgtgtcagcataggttatggttacgctgacag |
| | ggccagcgaaagcatcattggtgctatagcgcggcagttcaagttttcccgggtatgcaaaccgaaatcctcacttgaagagacg |
| | gaagttctgtttgtattcattgggtacgatcgcaaggcccgtacgcacaatccttacaagctttcatcaaccttgaccaacatttata |
| | caggttccagactccacgaagccggatgtgcaccctcatatcatgtggtgcgaggggatattgccacggccaccgaaggagtgat |
| | tataaatgctgctaacagcaaaggacaacctggcggaggggtgtgcggagcgctgtataagaaattcccggaaagcttcgattt |
| | acagccgatcgaagtaggaaaagcgcgactggtcaaaggtgcagctaaacatatcattcatgccgtaggaccaaacttcaacaa |
| | agtttcggaggttgaaggtgacaaacagttggcagaggcttatgagtccatcgctaagattgtcaacgataacaattacaagtcag |
| | tagcgattccactgttgtccaccggcatcttttccgggaacaaagatcgactaacccaatcattgaaccatttgctgacagctttaga |
| | caccactgatgcagatgtagccatatactgcagggacaagaaatgggaaatgactctcaaggaagcagtggctaggagagaag |
| | cagtggaggagatatgcatatccgacgactcttcagtgacagaacctgatgcagagctggtgagggtgcatccgaagagttcttt |
| | ggctggaaggaagggctacagcacaagcgatggcaaaactttctcatatttggaagggaccaagtttcaccaggcggccaagg |
| | atatagcagaaattaatgccatgtggcccgttgcaacggaggccaatgagcaggtatgcatgtatatcctcggagaaagcatgag |
| | cagtattaggtcgaaatgccccgtcgaagagtcggaagcctccacaccacctagcacgctgccttgcttgtgcatccatgccatg |
| | actccagaaagagtacagcgcctaaaagcctcacgtccagaacaaattactgtgtgctcatcctttccattgccgaagtatagaatc |
| | actggtgtgcagaagatccaatgctcccagcctatattgttctcaccgaaagtgcctgcgtatattcatccaaggaagtatctcgtg |
| | gaaacaccaccggtagacgagactccggagccatcggcagagaaccaatccacagaggggacacctgaacaaccaccactt |
| | ataaccgaggatgagaccaggactagaacgcctgagccgatcatcatcgaagaggaagaagaggatagcataagtttgctgtc |
| | agatggcccgacccaccaggtgctgcaagtcgaggcagacattcacgggccgccctctgtatctagctcatcctggtccattcct |
| | catgcatccgactttgatgtggacagtttatccatacttgacaccctggagggagctagcgtgaccagcggggcaacgtcagcc |
| | gagactaactcttacttcgcaaagagtatggagtttctggcgcgaccggtgcctgcgcctcgaacagtattcaggaaccctccac |
| | atcccgctccgcgcacaagaacaccgtcacttgcacccagcagggcctgctcgagaaccagcctagtttccaccccgccaggc |
| | gtgaatagggtgatcactagagaggagctcgaggcgcttaccccgtcacgcactcctagcaggtcggtctcgagaaccagcct |
| | ggtctccaacccgccaggcgtaaatagggtgattacaagagaggagtttgaggcgttcgtagcacaacaacaatgacggtttga |
| | tgcgggtgcatacatcttttcctccgacaccggtcaagggcatttacaacaaaaatcagtaaggcaaacggtgctatccgaagtg |
| | gtgttggagaggaccgaattggagatttcgtatgccccgcgcctcgaccaagaaaaagaagaattactacgcaagaaattacag |
| | ttaaatcccacacctgctaacagaagcagataccagtccaggaaggtggagaacatgaaagccataacagctagacgtattctg |
| | caaggcctagggcattatttgaaggcagaaggaaaagtggagtgctaccgaaccctgcatcctgttcctttgtattcatctagtgtg |
| | aaccgtgccttttcaagccccaaggtcgcagtggaagcctgtaacgccatgttgaaagagaactttccgactgtggcttcttactgt |
| | attattccagagtacgatgcctatttggacatggttgacggagcttcatgctgcttagacactgccagtttttgccctgcaaagctgc |
| | gcagctttccaaagaaacactcctatttggaacccacaatacgatcggcagtgccttcagcgatccagaacacgctccagaacgt |
| | cctggcagctgccacaaaaagaaattgcaatgtcacgcaaatgagagaattgcccgtattggattcggcggcctttaatgtggaat |
| | gcttcaagaaatatgcgtgtaataatgaatattgggaaacgtttaaagaaaaccccatcaggcttactgaagaaaacgtggtaaatt |
| | acattaccaaattaaaaggaccaaaagctgctgctctttttgcgaagacacataatttgaatatgttgcaggacataccaatggaca |
| | ggtttgtaatggacttaaagagagacgtgaaagtgactccaggaacaaaacatactgaagaacggcccaaggtacaggtgatcc |
| | aggctgccgatccgctagcaacagcgtatctgtgcggaatccaccgagagctggttaggagattaaatgcggtcctgcttccga |
| | acattcatacactgtttgatatgtcggctgaagactttgacgctattatagccgagcacttccagcctggggattgtgttctggaaac |
| | tgacatcgcgtcgtttgataaaagtgaggacgacgccatggctctgaccgcgttaatgattctggaagacttaggtgtggacgcag |
| | agctgttgacgctgattgaggcggctttcggcgaaatttcatcaatacatttgcccactaaaactaaatttaaattcggagccatgat |
| | gaaatctggaatgttcctcacactgtttgtgaacacagtcattaacattgtaatcgcaagcagagtgttgagagaacggctaaccg |
| | gatcaccatgtgcagcattcattggagatgacaatatcgtgaaaggagtcaaatcggacaaattaatggcagacaggtgcgcca |
| | cctggttgaatatggaagtcaagattatagatgctgtggtgggcgagaaagcgccttatttctgtggagggtttattttgtgtgactc |
| | cgtgaccggcacagcgtgccgtgtggcagaccccctaaaaaggctgtttaagcttggcaaacctctggcagcagacgatgaaca |
| | tgatgatgacaggagaagggcattgcatgaagagtcaacacgctggaaccgagtgggtattctttcagagctgtgcaaggcagt |
| | agaatcaaggtatgaaaccgtaggaacttccatcatagttatggccatgactactctagctagcagtgttaaatcattcagctacctg |
| | agaggggcccctataactctctacggctaacctgaatggactacgacatagtctagtccgccaagatggagaaaatagtgcttctt |
| | cttgcaatagtcagccttgttaaaagtgatcagatttgcattggttaccatgcaaacaactcgacagagcaggttgacacaataatg |
| | gaaaagaacgtcactgttacacacgcccaagacatactggaaaagacacacaacgggaaactctgcgatctagatggagtgaa |
| | gcctctaattttaagagattgtagtgtagctggatggctcctcgggaacccaatgtgtgacgaattcctcaatgtgccggaatggtct |
| | tacatagtggagaagatcaatccagccaatgacctctgttacccagggaatttcaacgactatgaagaactgaaacacctattgag |
| | cagaataaaccattttgagaaaattcagatcatccccaaaagttcttggtcagatcatgaagcctcagcaggggtgagctcagcat |
| | gtccataccagggaaggtcctccttttttagaaatgtggtatggcttatcaaaaaggacaatgcatacccaacaataaagagaagtt |
| | acaataataccaaccaagaagatcttttggtattgtgggggattcaccatccaaatgatgcggcagagcagacaaggctctatcaa |
| | aacccaactacctatatttccgttgggacatcaacactaaaccagagattggtaccaaaaatagccactagatctaaggtaaacgg |
| | gcaaagtggaaggatggagttcttttggacaattttaaaaccgaatgatgcaataaactttgagagtaatggaaatttcattgctcca |
| | gaaaatgcatacaaaattgtcaagaaaggggactcaacaattatgaaaagtgagttggaatatggtaactgcaacaccaagtgtc |
| | aaactccaataggggcgataaactctagtatgccattccacaacatccaccctctcaccatcggggaatgccccaaatatgtgaaa |
| | tcaagcagattagtccttgctactgggctcagaaatagccctcaacgagagacacgaggactatttggagctatagcaggttttat |
| | agagggaggatggggaatggtagatggttggtatgggtaccaccatagcaacgagcaggggagtgggtacgctgcagacaa |
| | agaatccactcaaaaggcaatagatggagtcaccaataaggtcaactcgatcattgacaaaatgaacactcagtttgaggctgttg |
| | gaagggaatttaataacttagaaaggagaatagaaaatttaaacaagaagatggaagacggattcctagatgtctggacttataat |
| | gctgaacttctggttctcatggaaaatgagagaactctagactttcatgactcaaatgtcaagaacctttacgacaaggtccgacta |
| | cagcttagggataatgcaaaggagcttggtaacggttgtttcgagttctatcacagatgtgataatgaatgtatggaaagtgtaaga |
| | aacggaacgtatgactacccgcagtattcagaagaagcaagattaaaaagagaggaaataagtggagtaaaattggaatcaata |
| | ggaacttaccaaatactgtcaatttattcaacagtggcgagctccctagcactggcaatcatggtggctggtctatctttatggatgt |
| | gctccaatggatcgttacaatgcagaatttgcatttaagggcccctataactctctacggctaacctgaatggactacgacatagtct |
| | agtccgccaagatgaatccaaatcagaagataataaccatcggatcaatctgtatggtaattggaatagttagcttaatgttacaaat |
| | tgggaacatgatctcaatatgggtcagtcattcaattcagacagggaatcaatgccaagctgaaccaatcagcaatactaaatttct |
| | tactgagaaagctgtggcttcagtaacattagcgggcaattcatctctttgccccattagcggatgggctgtatacagtaaggacaa |
| | cagtataaggatcggttccaggggggatgtgtttgttataagagagccgttcatctcatgctcccacttggaatgcagaactttcttt |
| | ttgactcagggagccttgctgaatgacaagcactccaatgggactgtcaaagacagaagccctcacagaacattaatgagttgtcc |
| | tgtgggtgaggctccctccccatataactcaaggtttgagtctgttgcttggtcagcaagtgcttgccatgatggcaccagttggttg |
| | acaattggaatttctggtccagacaatggggctgtggctgtattgaaatacaatggcataataacagacaccatcaagagttggag |
| | gaacaacatactgagaactcaagagtctgaatgtgcatgtgtaaatggctcttgctttactgtaatgactgatggaccaagtagtgg |
| | gcaggcatcatataagatcttcaaaatggaaaaagggaaagtggttaaatcagtcgaattggatgctcctaattatcactatgagga |
| | gtgctcctgttatcctgatgccggcgaaatcacatgtgtgtgcagggataattggcatggctcaaataggccatgggtatctttcaat |
| | caaaatttggagtatcaaataggatatatatgcagtggagttttcggagacaatccacgccccaatgatggaacaggtagttgtggt |
| | ccggtgttctctaacggggcatatggggtaaaagggttttcattcaaatacggcaatggtgtttggatcgggagaaccaaaagca |
| | ctaattccaggagcggctttgaaatgatttgggacccaaatgggtggactggaacggacagtagcttttcggtgaagcaagatat |
| | cgtagcaataactgattggtcaggatatagcgggagttttgtccagcatccagaactgacaggattagattgcataagaccttgttt |
| | ctgggttgagttaatcagagggcggcctaaagagagcacaatttggaccagtgggagcagcatatctttttgtggtgtaaatagtg |
| | acactgttagttggtcttggccagacggtgctgagttgccattcaccattgacaagtagggcgcgcccacccagcggccgcccg |
| | ctacgccccaatgatccgaccagcaaaactcgatgtacttccgaggaactgatgtgcataatgcatcaggctggtacattagatcc |
| | ccgcttaccgcgggcaatatagcaacactaaaaactcgatgtacttccgaggaagcgcagtgcataatgctgcgcagtgttgcca |
| | cataaccactatattaaccatttatctagcggacgccaaaaactcaatgtatttctgaggaagcgtggtgcataatgccacgcagcg |
| | tctgcataacttttattatttcttttattaatcaacaaaattttgtttttaacatttcaaaaaaaaaaaaaaaaaaaaaaaaaaaaaa |
| | aaaaaaagaagagcgtttaaacacgtgatatctggcctcatgggccttcctttcactgcccgctttccagtcgggaaacctgtcgtgc |
| | cagctgcattaacatggtcatagctgtttccttgcgtattgggcgctctccgcttcctcgctcactgactcgctgcgctcggtcgttc |
| | gggtaaagcctggggtgcctaatgagcaaaaggccagcaaaaggccaggaaccgtaaaaaggccgcgttgctggcgtttttccat |
| | aggctccgcccccctgacgagcatcacaaaaatcgacgctcaagtcagaggtggcgaaacccgacaggactataaagatacc |
| | aggcgtttccccctggaagctccctcgtgcgctctcctgttccgaccctgccgcttaccggatacctgtccgcctttctcccttcgg |
| | gaagcgtggcgctttctcatagctcacgctgtaggtatctcagttcggtgtaggtcgttcgctccaagctgggctgtgtgcacgaa |
| | ccccccgttcagcccgaccgctgcgccttatccggtaactatcgtcttgagtccaacccggtaagacacgacttatcgccactgg |
| | cagcagccactggtaacaggattagcagagcgaggtatgtaggcggtgctacagagttcttgaagtggtggcctaactacggct |
| | acactagaagaacagtatttggtatctgcgctctgctgaagccagttaccttcggaaaaagagttggtagctcttgatccggcaaa |
| | caaaccaccgctggtagcggtggtttttttgtttgcaggcagcagattacgcgcagaaaaaaaggatctcaagaagatcctttgat |
| | cttttctacggggtctgacgctcagtggaacgaaaactcacgttaagggattttggtcatgaatacacggtgcctgactgcgttagc |
| | aatttaactgtgataaactaccgcattaaagcttatcgatgataagctgtcaaacatgagaattcttagaaaaactcatcgagcatca |
| | aatgaaactgcaatttattcatatcaggattatcaataccatatttttgaaaaagccgtttctgtaatgaaggagaaaactcaccgag |
| | gcagttccataggatggcaagatcctggtatcggtctgcgattccgactcgtccaacatcaatacaacctattaatttcccctcgtca |
| | aaaataaggttatcaagtgagaaatcaccatgagtgacgactgaatccggtgagaatggcaaaagcttatgcatttctttccagactt |
| | gttcaacaggccagccattacgctcgtcatcaaaatcactcgcatcaaccaaaccgttattcattcgtgattgcgcctgagcgaga |
| | cgaaatacgcgatcgctgttaaaaggacaattacaaacaggaatcgaatgcaaccggcgcaggaacactgccagcgcatcaac |
| | aatattttcacctgaatcaggatattcttctaatacctggaatgctgttttcccggggatcgcagtggtgagtaaccatgcatcatca |
| | ggagtacggataaaatgcttgatggtcggaagaggcataaattccgtcagccagtttagtctgaccatctcatctgtaacatcattgg |
| | caacgctacctttgccatgtttcagaaacaactctggcgcatcgggcttcccatacaatcgatagattgtcgcacctgattgcccga |
| | cattatcgcgagcccatttatacccatataaatcagcatccatgttggaatttaatcgcggcctcgagcaagacgtttcccgttgaat |
| | atggctcataacaccccttgtattactgtttatgtaagcagacagttttattgttcatgagcggatacatatttgaatgtatttagaa |
| | aaataaacaaataggggttccgcgcacatttccccgaaaagtgccacctaaattgtaagcgttaatattttgttaaaattcgcgttaa |
| | atttttgttaaatcagctcattttttaaccaataggccgaaatcggcaaaatcccttataaatcaaaagaatagaccgagatagggtt |
| | gagtggccgctacagggcgctcccattcgccattcaggctgcgcaactgttgggaagggcgtttcggtgcgggcctcttcgctattac |
| | gccagctggcgaaagggggatgtgctgcaaggcgattaagttgggtaacgccagggttttcccagtcacacgcgtaatacgactca |
| | ctatag |

1. A method of preparing an enriched population of lipid nanoparticles (LNPs), comprising the steps of:
contacting a composition comprising a lipid nanoparticle (LNP) population and RNA with an anion exchanger under conditions such that the anion exchanger binds unencapsulated RNA; and
collecting an effluent produced by the anion exchanger to obtain the enriched population of LNPs.

2. The method of claim 1, wherein the unencapsulated RNA comprises free RNA, RNA that is associated with the surface of the LNP population and/or partially encapsulated RNA.

3. The method of claim 1, wherein the composition:
(i) has an ionic concentration of between 5 and 50 mM;
(ii) further comprises salt at a concentration of not greater than 100 mM;
(iii) has a conductivity of less than 15 mS/cm; and/or
(iv) further comprises a buffer selected from a citrate buffer, a bis-tris buffer, a histidine buffer, an acetate buffer, a phosphate buffer, a tris buffer and/or combinations thereof.

4-7. (canceled)

8. The method of claim 1, wherein each LNP in the LNP population comprises an ionizable lipid and one or more of a neutral lipid, a PEGylated lipid, and a structural lipid.

9-13. (canceled)

14. The method of claim 1, wherein each LNP in the LNP population comprises a lipid component comprising: about 25 mol % to about 60 mol % of an ionizable lipid; about 2 mol % to about 25 mol % of a neutral lipid; about 18.5 mol % to about 60 mol % of a structural lipid; and about 0.2 mol % to about 10 mol % of a PEGylated lipid.

15. The method of claim 1, wherein the RNA is a messenger RNA (mRNA) or a self-amplifying mRNA (sa-mRNA).

16-17. (canceled)

18. The method of claim 1, wherein the anion exchanger is anion exchange resin or an anion exchange membrane.

19. The method of claim 18, wherein the anion exchange membrane is a Mustang® Q, Sartobind Q, Chromasorb®, Capto Q, Q Sepharose Fast Flow (QSFF), Poros® Q, Fractogel® EMD, Natrix® Q, or Eshmuno® Q membrane.

20. The method of claim 19, wherein the anion exchange membrane is a Mustang® Q membrane.

21. The method of claim 1, further comprising eluting the unencapsulated RNA from the anion exchanger.

22. The method of claim 1, wherein each LNP in the LNP population has a diameter of about 30 nm to about 160 nm.

23. A composition comprising an enriched population of LNPs produced by the method of claim 1.

24. The composition of claim 23, wherein at least 90% of the RNA is encapsulated within the enriched population of LNPs.

25. An enriched lipid nanoparticle (LNP) composition comprising
(i) a plurality of lipid nanoparticles wherein each LNP comprises;
an ionizable lipid,
a neutral lipid,
a PEGylated lipid, and
a structural lipid; and
(ii) RNA;
wherein at least 90% of the RNA is encapsulated within the enriched LNP composition.

26. A pharmaceutical composition comprising an enriched population of LNPs produced by the method of claim 1, and a pharmaceutically acceptable carrier.

27. A pharmaceutical composition comprising the enriched LNP composition of claim 25, and a pharmaceutically acceptable carrier.

28. A method of delivering a RNA to a cell in a mammalian subject, comprising administering the pharmaceutical composition of claim 26 to the subject to thereby contact the cell with a lipid nanoparticle fully encapsulated with RNA and deliver the RNA to the cell.

29. (canceled)

30. A method of producing a polypeptide of interest in a mammalian cell, comprising contacting the cell with the pharmaceutical composition of claim 26.

31. A method of treating a disease, disorder or condition in a subject in need of such treatment, comprising administering the pharmaceutical composition of claim 26, to the subject to thereby treat the disease, disorder or condition, wherein the disease, disorder or condition is selected from the group consisting of a rare disease, an infectious disease, cancer, a proliferative disease, a genetic disease, an autoimmune disease, diabetes, a neurodegenerative disease, a cardiovascular disease, a reno-vascular disease, and a metabolic disease.

32-34. (canceled)

35. A vaccine comprising the composition of claim 23.

36. The vaccine of claim 35, wherein the vaccine is selected from a tumor vaccine, an influenza vaccine, a SARS vaccine, and a SARS-CoV-2 vaccine.

37-38. (canceled)


SEQUENCE LISTING (2 sequences)

SEQ ID NO: 1 - DNA, 11884 bp, synthetic construct
  source 1..11884: mol_type = other DNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatggagaaaatagtgcttcttcttgcaatagtcagccttgttaaaagtgatcagatttgcattggttaccatgcaaacaactcgacagagcaggttgacacaataatggaaaagaacgtcactgttacacacgcccaagacatactggaaaagacacacaacgggaaactctgcgatctagatggagtgaagcctctaattttaagagattgtagtgtagctggatggctcctcgggaacccaatgtgtgacgaattcctcaatgtgccggaatggtcttacatagtggagaagatcaatccagccaatgacctctgttacccagggaatttcaacgactatgaagaactgaaacacctattgagcagaataaaccattttgagaaaattcagatcatccccaaaagttcttggtcagatcatgaagcctcagcaggggtgagctcagcatgtccataccagggaaggtcctccttttttagaaatgtggtatggcttatcaaaaaggacaatgcatacccaacaataaagagaagttacaataataccaaccaagaagatcttttggtattgtgggggattcaccatccaaatgatgcggcagagcagacaaggctctatcaaaacccaactacctatatttccgttgggacatcaacactaaaccagagattggtaccaaaaatagccactagatctaaggtaaacgggcaaagtggaaggatggagttcttttggacaattttaaaaccgaatgatgcaataaactttgagagtaatggaaatttcattgctccagaaaatgcatacaaaattgtcaagaaaggggactcaacaattatgaaaagtgagttggaatatggtaactgcaacaccaagtgtcaaactccaataggggcgataaactctagtatgccattccacaacatccaccctctcaccatcggggaatgccccaaatatgtgaaatcaagcagattagtccttgctactgggctcagaaatagccctcaacgagagacacgaggactatttggagctatagcaggttttatagagggaggatggggaatggtagatggttggtatgggtaccaccatagcaacgagcaggggagtgggtacgctgcagacaaagaatccactcaaaaggcaatagatggagtcaccaataaggtcaactcgatcattgacaaaatgaacactcagtttgaggctgttggaagggaatttaataacttagaaaggagaatagaaaatttaaacaagaagatggaagacggattcctagatgtctggacttataatgctgaacttctggttctcatggaaaatgagagaactctagactttcatgactcaaatgtcaagaacctttacgacaaggtccgactacagcttagggataatgcaaaggagcttggtaacggttgtttcgagttctatcacagatgtgataatgaatgtatggaaagtgtaagaaacggaacgtatgactacccgcagtattcagaagaagcaagattaaaaagagaggaaataagtggagtaaaattggaatcaataggaacttaccaaatactgtcaatttattcaacagtggcgagctccctagcactggcaatcatggtggctggtctatctttatggatgtgctccaatggatcgttacaatgcagaatttgcatttaaggcgcgcccacccagcggccgcccgctacgccccaatgatccgaccagcaaaactcgatgtacttccgaggaactgatgtgcataatgcatcaggctggtacattagatccccgcttaccgcgggcaatatagcaacactaaaaactcgatgtacttccgaggaagcgcagtgcataatgctgcgcagtgttgccacataaccactatattaaccatttatctagcggacgccaaaaactcaatgtatttctgaggaagcgtggtgcataatgccacgcagcgtctgcataacttttattatttcttttattaatcaacaaaattttgtttttaacatttcaaaaaaaaaaaaaaaaaaaaaaaaaaaaaaaaaaaaagaagagcgtttaaacacgtgatatctggcctcatgggccttcctttcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaggcagcagattacgcgcagaaaaaaaggatctcaagaagatcctttgatcttttctacggggtctgacgctcagtggaacgaaaactcacgttaagggattttggtcatgaatacacggtgcctgactgcgttagcaatttaactgtgataaactaccgcattaaagcttatcgatgataagctgtcaaacatgagaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacacgcgtaatacgactcactatag

SEQ ID NO: 2 - DNA, 13295 bp, synthetic construct
  source 1..13295: mol_type = other DNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatggagaaaatagtgcttcttcttgcaatagtcagccttgttaaaagtgatcagatttgcattggttaccatgcaaacaactcgacagagcaggttgacacaataatggaaaagaacgtcactgttacacacgcccaagacatactggaaaagacacacaacgggaaactctgcgatctagatggagtgaagcctctaattttaagagattgtagtgtagctggatggctcctcgggaacccaatgtgtgacgaattcctcaatgtgccggaatggtcttacatagtggagaagatcaatccagccaatgacctctgttacccagggaatttcaacgactatgaagaactgaaacacctattgagcagaataaaccattttgagaaaattcagatcatccccaaaagttcttggtcagatcatgaagcctcagcaggggtgagctcagcatgtccataccagggaaggtcctccttttttagaaatgtggtatggcttatcaaaaaggacaatgcatacccaacaataaagagaagttacaataataccaaccaagaagatcttttggtattgtgggggattcaccatccaaatgatgcggcagagcagacaaggctctatcaaaacccaactacctatatttccgttgggacatcaacactaaaccagagattggtaccaaaaatagccactagatctaaggtaaacgggcaaagtggaaggatggagttcttttggacaattttaaaaccgaatgatgcaataaactttgagagtaatggaaatttcattgctccagaaaatgcatacaaaattgtcaagaaaggggactcaacaattatgaaaagtgagttggaatatggtaactgcaacaccaagtgtcaaactccaataggggcgataaactctagtatgccattccacaacatccaccctctcaccatcggggaatgccccaaatatgtgaaatcaagcagattagtccttgctactgggctcagaaatagccctcaacgagagacacgaggactatttggagctatagcaggttttatagagggaggatggggaatggtagatggttggtatgggtaccaccatagcaacgagcaggggagtgggtacgctgcagacaaagaatccactcaaaaggcaatagatggagtcaccaataaggtcaactcgatcattgacaaaatgaacactcagtttgaggctgttggaagggaatttaataacttagaaaggagaatagaaaatttaaacaagaagatggaagacggattcctagatgtctggacttataatgctgaacttctggttctcatggaaaatgagagaactctagactttcatgactcaaatgtcaagaacctttacgacaaggtccgactacagcttagggataatgcaaaggagcttggtaacggttgtttcgagttctatcacagatgtgataatgaatgtatggaaagtgtaagaaacggaacgtatgactacccgcagtattcagaagaagcaagattaaaaagagaggaaataagtggagtaaaattggaatcaataggaacttaccaaatactgtcaatttattcaacagtggcgagctccctagcactggcaatcatggtggctggtctatctttatggatgtgctccaatggatcgttacaatgcagaatttgcatttaagggcccctataactctctacggctaacctgaatggactacgacatagtctagtccgccaagatgaatccaaatcagaagataataaccatcggatcaatctgtatggtaattggaatagttagcttaatgttacaaattgggaacatgatctcaatatgggtcagtcattcaattcagacagggaatcaatgccaagctgaaccaatcagcaatactaaatttcttactgagaaagctgtggcttcagtaacattagcgggcaattcatctctttgccccattagcggatgggctgtatacagtaaggacaacagtataaggatcggttccaggggggatgtgtttgttataagagagccgttcatctcatgctcccacttggaatgcagaactttctttttgactcagggagccttgctgaatgacaagcactccaatgggactgtcaaagacagaagccctcacagaacattaatgagttgtcctgtgggtgaggctccctccccatataactcaaggtttgagtctgttgcttggtcagcaagtgcttgccatgatggcaccagttggttgacaattggaatttctggtccagacaatggggctgtggctgtattgaaatacaatggcataataacagacaccatcaagagttggaggaacaacatactgagaactcaagagtctgaatgtgcatgtgtaaatggctcttgctttactgtaatgactgatggaccaagtagtgggcaggcatcatataagatcttcaaaatggaaaaagggaaagtggttaaatcagtcgaattggatgctcctaattatcactatgaggagtgctcctgttatcctgatgccggcgaaatcacatgtgtgtgcagggataattggcatggctcaaataggccatgggtatctttcaatcaaaatttggagtatcaaataggatatatatgcagtggagttttcggagacaatccacgccccaatgatggaacaggtagttgtggtccggtgttctctaacggggcatatggggtaaaagggttttcattcaaatacggcaatggtgtttggatcgggagaaccaaaagcactaattccaggagcggctttgaaatgatttgggacccaaatgggtggactggaacggacagtagcttttcggtgaagcaagatatcgtagcaataactgattggtcaggatatagcgggagttttgtccagcatccagaactgacaggattagattgcataagaccttgtttctgggttgagttaatcagagggcggcctaaagagagcacaatttggaccagtgggagcagcatatctttttgtggtgtaaatagtgacactgttagttggtcttggccagacggtgctgagttgccattcaccattgacaagtagggcgcgcccacccagcggccgcccgctacgccccaatgatccgaccagcaaaactcgatgtacttccgaggaactgatgtgcataatgcatcaggctggtacattagatccccgcttaccgcgggcaatatagcaacactaaaaactcgatgtacttccgaggaagcgcagtgcataatgctgcgcagtgttgccacataaccactatattaaccatttatctagcggacgccaaaaactcaatgtatttctgaggaagcgtggtgcataatgccacgcagcgtctgcataacttttattatttcttttattaatcaacaaaattttgtttttaacatttcaaaaaaaaaaaaaaaaaaaaaaaaaaaaaaaaaaaaagaagagcgtttaaacacgtgatatctggcctcatgggccttcctttcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaggcagcagattacgcgcagaaaaaaaggatctcaagaagatcctttgatcttttctacggggtctgacgctcagtggaacgaaaactcacgttaagggattttggtcatgaatacacggtgcctgactgcgttagcaatttaactgtgataaactaccgcattaaagcttatcgatgataagctgtcaaacatgagaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacacgcgtaatacgactcactatag